SEQUENCE LISTING (33 sequences)

SEQ ID NO: 1 - DNA, 34 bp, Artificial Sequence
  Synthetic Sequence_Probe 1
gctgcccaaacgatggctgaaaaagggaggcagc

SEQ ID NO: 2 - DNA, 35 bp, Artificial Sequence
  Synthetic Sequence_Probe 2
gctgccaatcgcaaaagctgttcagtcacggcagc

SEQ ID NO: 3 - DNA, 40 bp, Artificial Sequence
  Synthetic Sequence_Probe 3
cgcgctacccagtcataacttactcaacagcaacagcgcg

SEQ ID NO: 4 - DNA, 45 bp, Artificial Sequence
  Synthetic Sequence_Probe 4
gctgccgtctcaaaatttgttgatcaagacaatacaattggcagc

SEQ ID NO: 5 - DNA, 32 bp, Artificial Sequence
  Synthetic Sequence_Probe 5
gctgccagggctttcaccgaagagggggcagc

SEQ ID NO: 6 - DNA, 35 bp, Artificial Sequence
  Synthetic Sequence_Probe 6
gctgccaacgaagtaggtggagacggaggggcagc

SEQ ID NO: 7 - DNA, 23 bp, Artificial Sequence
  Synthetic Sequence_Probe 7
ttgcaaattggcttgcccccact

SEQ ID NO: 8 - DNA, 35 bp, Artificial Sequence
  Synthetic Sequence_Probe 8
gcgagcccatgtgaattccctgcatcaatgctcgc

SEQ ID NO: 9 - DNA, 24 bp, Artificial Sequence
  Synthetic Sequence_Probe 9
accccgcattacgtttggtggacc

SEQ ID NO: 10 - DNA, 24 bp, Artificial Sequence
  Synthetic Sequence_Probe 10
aycacattggcacccgcaatcctg

SEQ ID NO: 11 - DNA, 23 bp, Artificial Sequence
  Synthetic Sequence_Probe 11
ttctgacctgaaggctctgcgcg

SEQ ID NO: 12 - DNA, 20 bp, Artificial Sequence
  Synthetic Sequence_forward primer 1
acctacaaggcaacaacatc

SEQ ID NO: 13 - DNA, 21 bp, Artificial Sequence
  Synthetic Sequence_forward primer 2
ccatttacctaagtgatggaa

SEQ ID NO: 14 - DNA, 20 bp, Artificial Sequence
  Synthetic Sequence_forward primer 3
ggagcattgtgtcatctgtc

SEQ ID NO: 15 - DNA, 20 bp, Artificial Sequence
  Synthetic Sequence_forward primer 4
cctggagtcccatcaaaagt

SEQ ID NO: 16 - DNA, 20 bp, Artificial Sequence
  Synthetic Sequence_forward primer 5
aaagcgaatttcagtgtgat

SEQ ID NO: 17 - DNA, 20 bp, Artificial Sequence
  Synthetic Sequence_forward primer 6
gtccatcaagctccagtttt

SEQ ID NO: 18 - DNA, 21 bp, Artificial Sequence
  Synthetic Sequence_forward primer 7
ccactactcccatttcgtcag

SEQ ID NO: 19 - DNA, 23 bp, Artificial Sequence
  Synthetic Sequence_forward primer 8
tttccacaatatytaagtgtcaa

SEQ ID NO: 20 - DNA, 20 bp, Artificial Sequence
  Synthetic Sequence_forward primer 9
gaccccaaaatcagcgaaat

SEQ ID NO: 21 - DNA, 22 bp, Artificial Sequence
  Synthetic Sequence_forward primer 10
gggagccttgaatacaccaaaa

SEQ ID NO: 22 - DNA, 19 bp, Artificial Sequence
  Synthetic Sequence_forward primer 11
agatttggacctgcgagcg

SEQ ID NO: 23 - DNA, 20 bp, Artificial Sequence
  Synthetic Sequence_reverse primer 1
cttcctgctggtgtgttaat

SEQ ID NO: 24 - DNA, 20 bp, Artificial Sequence
  Synthetic Sequence_reverse primer 2
cgtggcataatcttcttttt

SEQ ID NO: 25 - DNA, 20 bp, Artificial Sequence
  Synthetic Sequence_reverse primer 3
tagtgtgtaatgcagctcgt

SEQ ID NO: 26 - DNA, 21 bp, Artificial Sequence
  Synthetic Sequence_reverse primer 4
gcatctatacgaacacctgct

SEQ ID NO: 27 - DNA, 19 bp, Artificial Sequence
  Synthetic Sequence_reverse primer 5
gaaggcaatggtgagattt

SEQ ID NO: 28 - DNA, 20 bp, Artificial Sequence
  Synthetic Sequence_reverse primer 6
tcttcttacagcttgcttgc

SEQ ID NO: 29 - DNA, 26 bp, Artificial Sequence
  Synthetic Sequence_reverse primer 7
cagtatgtgtagtgcgcatataagca

SEQ ID NO: 30 - DNA, 22 bp, Artificial Sequence
  Synthetic Sequence_reverse primer 8
tcatcwccatacttttctgtta

SEQ ID NO: 31 - DNA, 24 bp, Artificial Sequence
  Synthetic Sequence_reverse primer 9
tctggttactgccagttgaatctg

SEQ ID NO: 32 - DNA, 21 bp, Artificial Sequence
  Synthetic Sequence_reverse primer 10
tgtagcacgattgcagcattg

SEQ ID NO: 33 - DNA, 20 bp, Artificial Sequence
  Synthetic Sequence_reverse primer 11
gagcggctgtctccacaagt

(19) United States
(12) Patent Application Publication
(10) Pub. No.: US 2024/0093319 A1
(43) Pub. Date: Mar. 21, 2024

(54) Methods for Detecting a Panel of Viruses Causing Respiratory Illness

(71) Applicant: Avellino Lab USA, Inc., Menlo Park, CA (US)

(72) Inventors: Connie Chao-Shern, Menlo Park, CA (US); Jun-Heok Jang, Menlo Park, CA (US)

(73) Assignee: Avellino Lab USA, Inc., Menlo Park, CA (US)

(21) Appl. No.: 18/275,137

(22) PCT Filed: Jan. 28, 2022
(86) PCT No.: PCT/US2022/014230

§ 371 Date: Jul. 31, 2023

Related U.S. Application Data
(60) Provisional application No. 63/143,283, filed on Jan. 29, 2021.

(51) Int. Cl. C12Q 1/70; C12Q 1/6844

(52) U.S. Cl. CPC C12Q 1/701; C12Q 1/6844; C12Q 1/6806

(57) ABSTRACT

The present invention provides compositions and methods for detecting and treating respiratory illness. The methods and compositions described herein comprise at least one probe that may hybridize with a target nucleotide molecule. The target nucleotide molecule may be a part of a virus, which in turn may be present in a subject, such as a human subject.

### BACKGROUND

Influenza, or flu, is a common viral respiratory infection that causes an illness ranging from mild to severe, and sometimes can be fatal. Influenza/flu tends to be seasonal, usually beginning in late fall and disappearing in early spring. According to the Centers for Disease Control and Prevention (CDC), influenza/flu affects millions of Americans each season. Signs and symptoms like headache, fever, chills, muscle pains, exhaustion, a stuffy nose, sore throat, and a cough tend to be more severe and longer lasting than the symptoms caused by the common cold. Two types of influenza virus, A and B, cause annual flu pandemics and most epidemics. Type C can cause mild respiratory illness and is not thought to cause epidemics. There are numerous subtypes of influenza A viruses.

Influence/flu and COVID-19 are both contagious respiratory illnesses, but they are caused by different viruses. COVID-19 is caused by infection with a new coronavirus (called SARS-CoV-2) and influenza/flu is caused by infection with influenza viruses. Influence/flu and COVID-19 share many characteristics, but there are some key differences between the two. COVID-19 seems to spread more easily than influenza/flu and causes more serious illnesses in some people. It can also take longer before people show symptoms and people can be contagious for longer. Another important difference is that there is a vaccine to protect against flu. The vaccines available to prevent COVID-19 are not widely available. Therefore, currently, the best way to prevent mass infection is still to avoid being exposed to the virus, e.g., through a combination of social distancing and diagnosis.

Because some of the symptoms of influenza/flu and COVID-19 are similar, it may be difficult to tell the difference between these respiratory illnesses based on symptoms alone, and testing may be needed to help confirm a diagnosis. Influenza/flu testing detects the presence of the virus in respiratory samples, more specifically, the M (Matrix) gene in influenza A, the NS gene in influenza B, and the NP gene in influenza C. These are highly conserved regions of the viruses. However, improved methods are necessary to distinguish between different types of influenza/flu and COVID-19 infections.

### SUMMARY

Accordingly, it is an object of the invention to provide compositions and methods for detecting and treating respiratory illness.

In one aspect, the present disclosure relates to a composition comprising at least one probe selected from the group consisting of probe 1, probe 2, probe 3, probe 4, probe 5, probe 6, probe 7, probe 8, probe 9, probe 10, and probe 11, wherein probe 1 comprises a nucleic acid sequence having at least 85% sequence identity to SEQ ID NO: 1, wherein probe 2 comprises a nucleic acid sequence having at least 85% sequence identity to SEQ ID NO: 2, wherein probe 3 comprises a nucleic acid sequence having at least 85% sequence identity to SEQ ID NO: 3, wherein probe 4 comprises a nucleic acid sequence having at least 85% sequence identity to SEQ ID NO: 4, wherein probe 5 comprises a nucleic acid sequence having at least 85% sequence identity to SEQ ID NO: 5, wherein probe 6 comprises a nucleic acid sequence having at least 85% sequence identity to SEQ ID NO: 6, wherein probe 7 comprises a nucleic acid sequence having at least 85% sequence identity to SEQ ID NO: 7, wherein probe 8 comprises a nucleic acid sequence having at least 85% sequence identity to SEQ ID NO: 8, wherein probe 9 comprises a nucleic acid sequence having at least 85% sequence identity to SEQ ID NO: 9, wherein probe 10 comprises a nucleic acid sequence having at least 85% sequence identity to SEQ ID NO: 10, wherein probe 11 comprises a nucleic acid sequence having at least 85% sequence identity to SEQ ID NO: 11.

In another aspect, the present disclosure relates to a method for detecting a respiratory illness, comprising: hybridizing a target nucleotide molecule in a sample with a composition comprising at least one probe selected from the group consisting of probe 1, probe 2, probe 3, probe 4, probe 5, probe 6, probe 7, probe 8, probe 9, probe 10, and probe 11, wherein probe 1 comprises a nucleic acid sequence having at least 85% sequence identity to SEQ ID NO: 1, wherein probe 2 comprises a nucleic acid sequence having at least 85% sequence identity to SEQ ID NO: 2, wherein probe 3 comprises a nucleic acid sequence having at least 85% sequence identity to SEQ ID NO: 3, wherein probe 4 comprises a nucleic acid sequence having at least 85% sequence identity to SEQ ID NO: 4, wherein probe 5 comprises a nucleic acid sequence having at least 85% sequence identity to SEQ ID NO: 5, wherein probe 6 comprises a nucleic acid sequence having at least 85% sequence identity to SEQ ID NO: 6, wherein probe 7 comprises a nucleic acid sequence having at least 85% sequence identity to SEQ ID NO: 7, wherein probe 8 comprises a nucleic acid sequence having at least 85% sequence identity to SEQ ID NO: 8, wherein probe 9 comprises a nucleic acid sequence having at least 85% sequence identity to SEQ ID NO: 9, wherein probe 10 comprises a nucleic acid sequence having at least 85% sequence identity to SEQ ID NO: 10, wherein probe 11 comprises a nucleic acid sequence having at least 85% sequence identity to SEQ ID NO: 11.

In another aspect, the present disclosure relates to a method for treating a respiratory illness, comprising detecting the respiratory illness according to any of the methods disclosed herein.

In some embodiments of each or any of the above or below mentioned embodiments, the composition further comprises at least one forward primer selected from the group consisting of forward primers comprising a nucleic acid sequence having at least 85% sequence identity to SEQ ID NO: 12, 13, 14, 15, 16, 17, 18, 19, 20, 21, or 22.

In some embodiments of each or any of the above or below mentioned embodiments, the composition further comprises at least one reverse primer selected from the group consisting of reverse primers comprising a nucleic acid sequence having at least 85% sequence identity to SEQ ID NO: 23, 24, 25, 26, 27, 28, 29, 30, 31, 32, or 33.

In some embodiments of each or any of the above or below mentioned embodiments, the composition further comprises at least one forward primer selected from the group consisting of forward primers comprising a nucleic acid sequence having 100% sequence identity to SEQ ID NO: 12, 13, 14, 15, 16, 17, 18, 19, 20, 21, or 22.

In some embodiments of each or any of the above or below mentioned embodiments, the composition further comprises at least one reverse primer selected from the group consisting of reverse primers comprising a nucleic acid sequence having 100% sequence identity to SEQ ID NO: 23, 24, 25, 26, 27, 28, 29, 30, 31, 32, or 33.

In some embodiments of each or any of the above or below mentioned embodiments, the composition comprises at least one probe selected from the group consisting of probe 1, probe 2, probe 3, probe 4, probe 5, probe 6, probe 7, probe 8, probe 9, and probe 10.

In some embodiments of each or any of the above or below mentioned embodiments, the composition further comprises at least one dye selected from the group consisting of JUN-QSY, ABY-QSY, FAM, VIC, and Cy5.

In some embodiments of each or any of the above or below mentioned embodiments, the composition comprises at least one probe selected from the group consisting of probe 1, probe 2, probe 3, probe 4, probe 5, probe 6, probe 7, probe 8, probe 9, probe 10, and probe 11, wherein probe 1 comprises a nucleic acid sequence having at least 100% sequence identity to SEQ ID NO: 1, wherein probe 2 comprises a nucleic acid sequence having at least 100% sequence identity to SEQ ID NO: 2, wherein probe 3 comprises a nucleic acid sequence having at least 100% sequence identity to SEQ ID NO: 3, wherein probe 4 comprises a nucleic acid sequence having at least 100% sequence identity to SEQ ID NO: 4, wherein probe 5 comprises a nucleic acid sequence having at least 100% sequence identity to SEQ ID NO: 5, wherein probe 6 comprises a nucleic acid sequence having at least 100% sequence identity to SEQ ID NO: 6, wherein probe 7 comprises a nucleic acid sequence having at least 100% sequence identity to SEQ ID NO: 7, wherein probe 8 comprises a nucleic acid sequence having at least 100% sequence identity to SEQ ID NO: 8, wherein probe 9 comprises a nucleic acid sequence having at least 100% sequence identity to SEQ ID NO: 9, wherein probe 10 comprises a nucleic acid sequence having at least 100% sequence identity to SEQ ID NO: 10, wherein probe 11 comprises a nucleic acid sequence having at least 100% sequence identity to SEQ ID NO: 11.

In some embodiments of each or any of the above or below mentioned embodiments, the respiratory illness is selected from the group consisting of Parainfluenza 1, Parainfluenza 2, Parainfluenza 3, Parainfluenza 4, Influenza A, Influenza B, MERS, RSV infection, COVID-19, and SARS.

### BRIEF DESCRIPTION OF THE DRAWINGS

FIG. 1 depicts the principle of nucleic acid extraction from a sample using the magnetic beads method.

FIG. 2 shows exponential phase measurement in Real-Time PCR.

FIG. 3 shows an example of a PCR Plate Map, showing placement for extracted RNA samples, controls, and NTCs.

FIG. 4 shows analysis after PCR amplification, wherein thresholds are adjusted to fall within exponential phase of the fluorescence curves and above any background signal.

FIG. 5 shows a accuracy test plate layout.

FIG. 6 shows a precision test plate layout.

FIG. 7 shows an analytical sensitivity plate layout.

### DETAILED DESCRIPTION

In one aspect, the present disclosure relates generally to compositions and methods for detecting and treating respiratory illness. The methods and compositions described herein comprise at least one probe that may hybridize with a target nucleotide molecule. The target nucleotide molecule may be a part of a virus, which in turn may be present in a subject, such as a human subject. The virus may be in tissue, and/or in saliva, blood, and/or other fluids of the subject. The compositions described herein may further comprise at least one forward primer, at least one reverse primer, and/or at least one dye. The methods and compositions described herein may be used to detect and/or treat a respiratory illness, such as Parainfluenza 1, Parainfluenza 2, Parainfluenza 3, Parainfluenza 4, Influenza A, Influenza B, MERS, RSV infection, COVID-19, and SARS.

### I. DEFINITIONS

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using well known techniques. While there are several methods to measure identity between two polynucleotide or protein sequences, the term “identity” is well known to skilled artisans (Carillo (1988) J. Applied Math. 48, 1073). Examples of computer program methods to determine identity and similarity between two sequences include, but are not limited to, GCG program package (Devereux (1984) Nucleic Acids Research 12, 387), BLASTP, ExPASy, BLASTN, FASTA (Atschul (1990) J. Mol. Biol. 215, 403) and FASTDB. Examples of methods to determine identity and similarity are discussed in Michaels (2011) Current Protocols in Protein Science, Vol. 1, John Wiley & Sons.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventive treatment (treatment directed to minimizing or partially or completely inhibiting the development of the associated disease, pathological condition, or disorder), and supportive treatment (treatment employed to supplement another therapy). Treatment also includes diminishment of the extent of the disease or condition; preventing spread of the disease or condition; delaying or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or the time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera. When the term “about” is used, it is used to mean a certain effect or result can be obtained within a certain tolerance, and the skilled person knows how to obtain the tolerance. In some embodiments, the term “about” refers to a range of values that fall within 10, 9, 8, 7, 6, 5, 4, 3, 2, 1 percent or less of the stated reference value.

### II. COMPOSITIONS

The present disclosure relates to compositions and methods for detecting and treating respiratory illness. The methods and compositions described herein comprise at least one probe that may hybridize with a target nucleotide molecule and may further comprise at least one forward primer, at least one reverse primer, and/or at least one dye.

In one aspect, the present disclosure relates to a composition comprising at least one probe selected from the group consisting of probe 1, probe 2, probe 3, probe 4, probe 5, probe 6, probe 7, probe 8, probe 9, probe 10, and probe 11, wherein probe 1 comprises a nucleic acid sequence having at least 85% sequence identity to SEQ ID NO: 1, wherein probe 2 comprises a nucleic acid sequence having at least 85% sequence identity to SEQ ID NO: 2, wherein probe 3 comprises a nucleic acid sequence having at least 85% sequence identity to SEQ ID NO: 3, wherein probe 4 comprises a nucleic acid sequence having at least 85% sequence identity to SEQ ID NO: 4, wherein probe 5 comprises a nucleic acid sequence having at least 85% sequence identity to SEQ ID NO: 5, wherein probe 6 comprises a nucleic acid sequence having at least 85% sequence identity to SEQ ID NO: 6, wherein probe 7 comprises a nucleic acid sequence having at least 85% sequence identity to SEQ ID NO: 7, wherein probe 8 comprises a nucleic acid sequence having at least 85% sequence identity to SEQ ID NO: 8, wherein probe 9 comprises a nucleic acid sequence having at least 85% sequence identity to SEQ ID NO: 9, wherein probe 10 comprises a nucleic acid sequence having at least 85% sequence identity to SEQ ID NO: 10, wherein probe 11 comprises a nucleic acid sequence having at least 85% sequence identity to SEQ ID NO: 11.

In some embodiments, the at least one probe is probe 1. In some embodiments, the at least one probe is probe 2. In some embodiments, the at least one probe is probe 3. In some embodiments, the at least one probe is probe 4. In some embodiments, the at least one probe is probe 5. In some embodiments, the at least one probe is probe 6. In some embodiments, the at least one probe is probe 7. In some embodiments, the at least one probe is probe 8. In some embodiments, the at least one probe is probe 9. In some embodiments, the at least one probe is probe 10. In some embodiments, the at least one probe is probe 11.

In some embodiments, two probes are selected from the group consisting of probe 1, probe 2, probe 3, probe 4, probe 5, probe 6, probe 7, probe 8, probe 9, probe 10, and probe 11. In some embodiments, three probes are selected from the group consisting of probe 1, probe 2, probe 3, probe 4, probe 5, probe 6, probe 7, probe 8, probe 9, probe 10, and probe 11. In some embodiments, four probes are selected from the group consisting of probe 1, probe 2, probe 3, probe 4, probe 5, probe 6, probe 7, probe 8, probe 9, probe 10, and probe 11. In some embodiments, five probes are selected from the group consisting of probe 1, probe 2, probe 3, probe 4, probe 5, probe 6, probe 7, probe 8, probe 9, probe 10, and probe 11. In some embodiments, six probes are selected from the group consisting of probe 1, probe 2, probe 3, probe 4, probe 5, probe 6, probe 7, probe 8, probe 9, probe 10, and probe 11. In some embodiments, seven probes are selected from the group consisting of probe 1, probe 2, probe 3, probe 4, probe 5, probe 6, probe 7, probe 8, probe 9, probe 10, and probe 11. In some embodiments, eight probes are selected from the group consisting of probe 1, probe 2, probe 3, probe 4, probe 5, probe 6, probe 7, probe 8, probe 9, probe 10, and probe 11. In some embodiments, nine probes are selected from the group consisting of probe 1, probe 2, probe 3, probe 4, probe 5, probe 6, probe 7, probe 8, probe 9, probe 10, and probe 11. In some embodiments, ten probes are selected from the group consisting of probe 1, probe 2, probe 3, probe 4, probe 5, probe 6, probe 7, probe 8, probe 9, probe 10, and probe 11. In some embodiments, eleven probes are selected from the group consisting of probe 1, probe 2, probe 3, probe 4, probe 5, probe 6, probe 7, probe 8, probe 9, probe 10, and probe 11.

In another aspect, the present disclosure relates to a composition comprising at least one probe selected from the group consisting of probe 1, probe 2, probe 3, probe 4, probe 5, probe 6, probe 7, probe 8, probe 9, probe 10, and probe 11, wherein probe 1 comprises a nucleic acid sequence having at least 100% sequence identity to SEQ ID NO: 1, wherein probe 2 comprises a nucleic acid sequence having at least 100% sequence identity to SEQ ID NO: 2, wherein probe 3 comprises a nucleic acid sequence having at least 100% sequence identity to SEQ ID NO: 3, wherein probe 4 comprises a nucleic acid sequence having at least 100% sequence identity to SEQ ID NO: 4, wherein probe 5 comprises a nucleic acid sequence having at least 100% sequence identity to SEQ ID NO: 5, wherein probe 6 comprises a nucleic acid sequence having at least 100% sequence identity to SEQ ID NO: 6, wherein probe 7 comprises a nucleic acid sequence having at least 100% sequence identity to SEQ ID NO: 7, wherein probe 8 comprises a nucleic acid sequence having at least 100% sequence identity to SEQ ID NO: 8, wherein probe 9 comprises a nucleic acid sequence having at least 100% sequence identity to SEQ ID NO: 9, wherein probe 10 comprises a nucleic acid sequence having at least 100% sequence identity to SEQ ID NO: 10, wherein probe 11 comprises a nucleic acid sequence having at least 100% sequence identity to SEQ ID NO: 11.

In some embodiments, the composition described herein comprises probe 1. In additional embodiments, probe 1 may hybridize to a target nucleotide molecule that is a part of Human Parainfluenza Virus 1 (HPIV-1), causing Parainfluenza 1. In some embodiments, probe 1 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 1. In some embodiments, probe 1 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 1.

In some embodiments, the composition described herein comprises probe 2. In additional embodiments, probe 2 may hybridize to a target nucleotide molecule that is a part of Human Parainfluenza Virus 2 (HPIV-2), causing Parainfluenza 2. In some embodiments, probe 2 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 2. In some embodiments, probe 2 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 2.

In some embodiments, the composition described herein comprises probe 3. In additional embodiments, probe 3 may hybridize to a target nucleotide molecule that is a part of Human Parainfluenza Virus 3 (HPIV-3), causing Parainfluenza 3. In some embodiments, probe 3 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 3. In some embodiments, probe 3 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 3.

In some embodiments, the composition described herein comprises probe 4. In additional embodiments, probe 4 may hybridize to a target nucleotide molecule that is a part of Human Parainfluenza Virus 4 (HPIV-4), causing Parainfluenza 4. In some embodiments, probe 4 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 4. In some embodiments, probe 4 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 4.

In some embodiments, the composition described herein comprises probe 5. In additional embodiments, probe 5 may hybridize to a target nucleotide molecule that is a part of Influenza A virus, causing Influenza A. In some embodiments, probe 5 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 5. In some embodiments, probe 5 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 5. Table 1 shows potential target nucleotide molecules in the Influenza A virus.

TABLE 1
Potential target nucleotide molecules in the Influenza A virus

| | Max | Total | Query | E | Per. | |
|---|---|---|---|---|---|---|
| Description | Score | Score | Cover | value | ident | Accession |
| Influenza A virus | 52.8 | 52.8 | 40% | 5.00E− | 100 | MK995887.1 |
| (A/piegon/China/piegon/2017(H9N2)) segment | | | | 06 | | |
| 7 matrix protein 2 (M2) and matrix protein 1 | | | | | | |
| (M1) genes, complete cds | | | | | | |
| Influenza A virus | 52.8 | 52.8 | 40% | 5.00E− | 100 | MK367649.1 |
| (A/chicken/China/16587.7/2018(H9N2)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MK367633.1 |
| (A/chicken/China/1646.7/2018(H9N2)) segment | | | | 06 | | |
| 7 matrix protein 2 (M2) and matrix protein 1 | | | | | | |
| (M1) genes, complete cds | | | | | | |
| Influenza A virus | 52.8 | 52.8 | 40% | 5.00E− | 100 | MK367623.1 |
| (A/chicken/China/1642.5/2018(H9N2)) segment | | | | 06 | | |
| 7 matrix protein 2 (M2) and matrix protein 1 | | | | | | |
| (M1) genes, complete cds | | | | | | |
| Influenza A virus (A/duck/Viet Nam/QN- | 52.8 | 52.8 | 40% | 5.00E− | 100 | MK964725.1 |
| 0505/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK964497.1 |
| 0514/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK964472.1 |
| 0469/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK964440.1 |
| 0277/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK964408.1 |
| 0258/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK964399.1 |
| 0226/2013(mixed)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK 964357.1 |
| 0132/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/LS- | 52.8 | 52.8 | 40% | 5.00E− | 100 | MK964316.1 |
| 0170/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/HN- | 52.8 | 52.8 | 40% | 5.00E− | 100 | MK964196.1 |
| 0337/2013(H9N2)) segment 7 matrix protein 2 | | | | 06 | | |
| (M2) and matrix protein 1 (M1) genes, complete | | | | | | |
| cds | | | | | | |
| Influenza A virus (A/muscovy duck/Viet | 52.8 | 52.8 | 40% | 5.00E− | 100 | MK964163.1 |
| Nam/HN-0343/2013(mixed)) segment 7 matrix | | | | 06 | | |
| protein 2 (M2) and matrix protein 1 (M1) genes, | | | | | | |
| complete cds | | | | | | |
| Influenza A virus (A/chicken/Viet Nam/LS- | 52.8 | 52.8 | 40% | 5.00E− | 100 | MK964120.1 |
| 0160/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK964042.1 |
| 0136/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK964011.1 |
| 0141/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963958.1 |
| 0254/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963929.1 |
| 0452/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963845.1 |
| 0204/2013(mixed)) segment 7 matrix protein 2 | | | | 06 | | |
| (M2) and matrix protein 1 (M1 genes, complete | | | | | | |
| cds | | | | | | |
| Influenza A virus (A/chicken/Viet Nam/QN- | 52.8 | 52.8 | 40% | 5.00E− | 100 | MK963786.1 |
| 0472/2013(H9N2)) segment 7 matrix protein 2 | | | | 06 | | |
| (M2) and matrix protein 1 (M1 genes, complete | | | | | | |
| cds | | | | | | |
| Influenza A virus (A/muscovy duck/Viet | 52.8 | 52.8 | 40% | 5.00E− | 100 | MK963697.1 |
| Nam/QN-0224/2013(mixed)) segment 7 matrix | | | | 06 | | |
| protein 2 (M2) and matrix protein 1 (M1) genes, | | | | | | |
| complete cds | | | | | | |
| Influenza A virus (A/chicken/Viet Nam/LS- | 52.8 | 52.8 | 40% | 5.00E− | 100 | MK963654.1 |
| 0185/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/HN- | 52.8 | 52.8 | 40% | 5.00E− | 100 | MK963582.1 |
| 0334/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/LS- | 52.8 | 52.8 | 40% | 5.00E− | 100 | MK963548.1 |
| 0161/2013(mixed)) segment 7 matrix protein 2 | | | | 06 | | |
| (M2) and matrix protein 1 (M1) genes, complete | | | | | | |
| cds | | | | | | |
| Influenza A virus (A/duck/Viet Nam/QN- | 52.8 | 52.8 | 40% | 5.00E− | 100 | MK963543.1 |
| 0457/2013(mixed)) clone variant a segment 7 | | | | 06 | | |
| matrix protein 2 (M2) and matrix protein 1 (M1) | | | | | | |
| genes, complete cds | | | | | | |
| Influenza A virus (A/chicken/Viet Nam/QN- | 52.8 | 52.8 | 40% | 5.00E− | 100 | MK963532.1 |
| 0455/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/LS- | 52.8 | 52.8 | 40% | 5.00E− | 100 | MK963523.1 |
| 0120/2013(H9)) segment 7 matrix protein 2 | | | | 06 | | |
| (M2) and matrix protein 1 (M1) genes, complete | | | | | | |
| cds | | | | | | |
| Influenza A virus (A/duck/Viet Nam/QN- | 52.8 | 52.8 | 40% | 5.00E− | 100 | MK963476.1 |
| 0510/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963468.1 |
| 0512/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963458.1 |
| 0218/2013(mixed)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963419.1 |
| 0473/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963404.1 |
| 0271/2013(H5N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963387.1 |
| 0133/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/HN- | 52.8 | 52.8 | 40% | 5.00E− | 100 | MK963343.1 |
| 0598/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963310.1 |
| 0131/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/QN- | 52.8 | 52.8 | 40% | 5.00E− | 100 | MK963277.1 |
| 0144/2013(mixed)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/LS- | 52.8 | 52.8 | 40% | 5.00E− | 100 | MK963243.1 |
| 0171/2013(mixed)) clone variant a segment 7 | | | | 06 | | |
| matrix protein 2 (M2) and matrix protein 1 (M1) | | | | | | |
| genes, complete cds | | | | | | |
| Influenza A virus (A/chicken/Viet Nam/LS- | 52.8 | 52.8 | 40% | 5.00E− | 100 | MK963163.1 |
| 0187/2013(H9N2)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/HN- | 52.8 | 52.8 | 40% | 5.00E− | 100 | MK943234.1 |
| 2630/2016(H6N6)) segment 7 matrix protein 2 | | | | 06 | | |
| (M2) and matrix protein 1 (M1) genes, complete | | | | | | |
| cds | | | | | | |
| Influenza A virus (A/chicken/Viet Nam/HN- | 52.8 | 52.8 | 40% | 5.00E− | 100 | MK943218.1 |
| 2627/2016(H9N2)) segment 7 matrix protein 2 | | | | 06 | | |
| (M2) and matrix protein 1 (M1) genes, complete | | | | | | |
| cds | | | | | | |
| Influenza A virus | 52.8 | 52.8 | 40% | 5.00E− | 100 | MK744010.1 |
| (A/duck/Jiangxi/120/2011(H5N1)) segment 7 | | | | 06 | | |
| matrix protein 2 (M2) and matrix protein 1 (M1) | | | | | | |
| genes, complete cds | | | | | | |
| Influenza A virus | 52.8 | 52.8 | 40% | 5.00E− | 100 | MG575616.1 |
| (A/chicken/Hebei/SJ27/2017(H7N9)) segment 7 | | | | 06 | | |
| matrix protein 2 (M2) and matrix protein 1 (M1) | | | | | | |
| genes, complete cds | | | | | | |
| Influenza A virus | 52.8 | 52.8 | 40% | 5.00E− | 100 | MG575615.1 |
| (A/chicken/Guangxi/GX102/2017(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MG575614.1 |
| (A/chicken/Henan/HNXY 1/2017(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MG575613.1 |
| (A/chicken/Guangxi/GX110/2017(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MG575611.1 |
| (A/chicken/Jiangsu/JS/02/71/2017(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MG575610.1 |
| (A/chicken/Jiangsu/TM261/2017(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MG575609.1 |
| (A/chicken/Anhui/AH395/2017(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MG575608.1 |
| (A/chicken/Anhui/AH388/2017(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MK250036.1 |
| (A/chicken/Qingyuan/zd201602/2016(H9N2)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MK250000.1 |
| (A/chicken/Qingyuan/zd201601/2016(H9N2)) | | | | 06 | | |
| segment 7 matrix protein 2 (M2) and matrix | | | | | | |
| protein 1 (M1) genes, complete cds | | | | | | |
| Influenza A virus (A/duck/Southern | 52.8 | 52.8 | 40% | 5.00E− | 100 | MH114017.1 |
| China/04/2017(H7N9)) segment 7 matrix | | | | 06 | | |
| protein 2 (M2) and matrix protein 1 (M1) genes, | | | | | | |
| complete cds | | | | | | |
| Influenza A virus (A/Anthropoides | 52.8 | 52.8 | 40% | 5.00E− | 100 | MG871421.1 |
| virgo/Baicheng/220/2014(H9N2)) segment 7 | | | | 06 | | |
| matrix protein 2 (M2) and matrix protein 1 (M1) | | | | | | |
| genes, complete cds | | | | | | |
| Influenza A virus | 52.8 | 52.8 | 40% | 5.00E− | 100 | MF465803.1 |
| (A/dove/Guangxi/96B8/2014(H9N2)) segment 7 | | | | 06 | | |
| matrix protein 2 (M2) and matrix protein 1 (M1) | | | | | | |
| genes, complete cds | | | | | | |
| Influenza A virus (A/oriental magpie | 52.8 | 52.8 | 40% | 5.00E− | 100 | KX638419.1 |
| robin/HK/6154/2015(H5N6)) segment 7 matrix | | | | 06 | | |
| protein 2 (M2) and matrix protein 1 (M1) genes, | | | | | | |
| complete cds | | | | | | |
| Influenza A virus | 52.8 | 52.8 | 40% | 5.00E− | 100 | MF425648.1 |
| (A/quail/Guangxi/198Q39/2015(H9N2)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | MF319555.1 |
| (A/chicken/Heinan/ZZ01/2017(H7N9)) segment | | | | 06 | | |
| 7 matrix protein 2 (M2) and matrix protein 1 | | | | | | |
| (M1) genes, complete cds | | | | | | |
| Influenza A virus (A/Changsha/70/2017(H7N9)) | 52.8 | 52.8 | 40% | 5.00E− | 100 | MF370265.1 |
| segment 7 matrix protein 2 (M2) and matrix | | | | 06 | | |
| protein 1 (M1) genes, partial cds | | | | | | |
| Influenza A virus (A/Changsha/58/2017(H7N9)) | 52.8 | 52.8 | 40% | 5.00E− | 100 | MF370261.1 |
| segment 7 matrix protein 1 (M1) and matrix | | | | 06 | | |
| protein 2 (M2) genes, partial cds | | | | | | |
| Influenza A virus (A/Changsha/44/2017(H7N9)) | 52.8 | 52.8 | 40% | 5.00E− | 100 | MF370257.1 |
| segment 7 matrix protein 2 (M2) and matrix | | | | 06 | | |
| protein 1 (M1 genes, complete cds | | | | | | |
| Influenza A virus (A/Changsha/26/2017(H7N9)) | 52.8 | 52.8 | 40% | 5.00E− | 100 | MF370249.1 |
| segment 7 matrix protein 2 (M2) and matrix | | | | 06 | | |
| protein 1 (Ml1genes, complete cds | | | | | | |
| Influenza A virus | 52.8 | 52.8 | 40% | 5.00E− | 100 | MF164422.1 |
| (A/chicken/Zhejiang/HZ26/2014(H9N2)) | | | | 06 | | |
| segment 7 matrix protein 2 (M2) gene, partial | | | | | | |
| cds; and matrix protein 1 (M1) gene, complete | | | | | | |
| cds | | | | | | |
| Influenza A virus | 52.8 | 52.8 | 40% | 5.00E− | 100 | MF164421.1 |
| (A/duck/Anhui/AQ14/2014(H9N2)) segment 7 | | | | 06 | | |
| matrix protein 2 (M2) gene, partial cds; and | | | | | | |
| matrix protein 1 (M1) gene, complete cds | | | | | | |
| Influenza A virus | 52.8 | 52.8 | 40% | 5.00E− | 100 | MF164418.1 |
| (A/chicken/Zhejiang/HZ03/2014(H9N2)) | | | | 06 | | |
| segment 7 matrix protein 2 (M2) gene, partial | | | | | | |
| cds; and matrix protein 1 (M1) gene, complete | | | | | | |
| cds | | | | | | |
| Influenza A virus (A/Zhejiang/DTID- | 52.8 | 52.8 | 40% | 5.00E− | 100 | MF039637.1 |
| ZJU01/2013(H7N9)) segment 7 matrix protein 2 | | | | 06 | | |
| (M2) and matrix protein 1 (M1) genes, complete | | | | | | |
| cds | | | | | | |
| Influenza A virus | 52.8 | 52.8 | 40% | 5.00E− | 100 | KY910838.1 |
| (A/Yunnan/0129/2017(H7N9)) segment 7 | | | | 06 | | |
| matrix protein 2 (M2) and matrix protein 1 (M1) | | | | | | |
| genes, complete cds | | | | | | |
| Influenza A virus | 52.8 | 52.8 | 40% | 5.00E− | 100 | KY643850.1 |
| (A/Guangdong/SP440/2017(H7N9)) segment 7 | | | | 06 | | |
| matrix protein 2 (M2) and matrix protein 1 (M1) | | | | | | |
| genes, complete cds | | | | | | |
| Influenza A virus | 52.8 | 52.8 | 40% | 5.00E− | 100 | KY643849.1 |
| (A/Guangdong/HP001/2017(H7N9)) segment 7 | | | | 06 | | |
| matrix protein 2 (M2) and matrix protein 1 (M1) | | | | | | |
| genes, complete cds | | | | | | |
| Influenza A virus | 52.8 | 52.8 | 40% | 5.00E− | 100 | KY608242.1 |
| (A/goose/Jiangsu/SQ119/2012(H9N2)) segment | | | | 06 | | |
| 7 matrix protein 2 (M2) and matrix protein 1 | | | | | | |
| (M1) genes, complete cds | | | | | | |
| Influenza A virus | 52.8 | 52.8 | 40% | 5.00E− | 100 | KY608234.1 |
| (A/goose/Jiangsu/YZ527/2011(H9N2)) segment | | | | 06 | | |
| 7 matrix protein 2 (M2) and matrix protein 1 | | | | | | |
| (M1) genes, complete cds | | | | | | |
| Influenza A virus | 52.8 | 52.8 | 40% | 5.00E− | 100 | KY621545.1 |
| (A/Qingyuan/GIRD01/2017(H7N9)) segment 7 | | | | 06 | | |
| matrix protein 2 (M2) and matrix protein 1 (M1) | | | | | | |
| genes, complete cds | | | | | | |
| Influenza A virus | 52.8 | 52.8 | 40% | 5.00E− | 100 | KY056312.1 |
| (A/chicken/Zhejiang/221/2016(H9N2)) segment | | | | 06 | | |
| 7 matrix protein 2 (M2) and matrix protein 1 | | | | | | |
| (M1) genes, complete cds | | | | | | |
| Influenza A virus | 52.8 | 52.8 | 40% | 5.00E− | 100 | KX808591.1 |
| (A/Guangdong/MZ058/2016(H9N2)) segment 7 | | | | 06 | | |
| matrix protein 2 (M2) and matrix protein 1 (M1) | | | | | | |
| genes, complete cds | | | | | | |
| Influenza A virus (A/duck/Japan/AQ- | 52.8 | 52.8 | 40% | 5.00E− | 100 | LC208506.1 |
| HE5/2015(H9N2)) M2, M1 genes for M2 ion | | | | 06 | | |
| channel, matrix protein 1, complete cds | | | | | | |
| Influenza A virus (A/chicken/Japan/AQ- | 52.8 | 52.8 | 40% | 5.00E− | 100 | LC208490.1 |
| HE79/2015(H5N1)) M2, M1 genes for M2 ion | | | | 06 | | |
| channel, matrix protein 1, complete cds | | | | | | |
| Influenza A virus (A/chicken/Japan/AQ- | 52.8 | 52.8 | 40% | 5.00E− | 100 | LC208482.1 |
| HE61/2015(H9N2)) M2, M1 genes for M2 ion | | | | 06 | | |
| channel, matrix protein 1, complete cds | | | | | | |
| Influenza A virus | 52.8 | 52.8 | 40% | 5.00E− | 100 | KY415690.1 |
| (A/chicken/Ganzhou/GZ50/2015(H5N6)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415687.1 |
| (A/chicken/Ganzhou/GZ126/2016(H9N2)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415686.1 |
| (A/chicken/Ganzhou/GZ140/2016(H9N2)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415685.1 |
| (A/chicken/Ganzhou/GZ79/2016(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415684.1 |
| (A/duck/Ganzhou/GZ188/2016(H9N2)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415677.1 |
| (A/chicken/Taishun/TS2/2016(H5N6)) segment | | | | 06 | | |
| 7 matrix protein 2 (M2) and matrix protein 1 | | | | | | |
| (M1) genes, complete cds | | | | | | |
| Influenza A virus | 52.8 | 52.8 | 40% | 5.00E− | 100 | KY415676.1 |
| (A/chicken/Taishun/TS90/2016(H5N6)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415675.1 |
| (A/chicken/Taishun/TS12/2016(H5N6)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415674.1 |
| (A/chicken/Longquan/LQ78/2016(H7N9)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415673.1 |
| (A/chicken/Yuhuan/YH15/2016(H9N2)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415662.1 |
| (A/duck/Hubei/ZYSYF25/2016(H5N1)) | | | | 06 | | |
| segment 7 matrix protein 2 (M2) and matrix | | | | | | |
| protein 1 (M1) genes, complete cds | | | | | | |
| Influenza A virus | 52.8 | 52.8 | 40% | 5.00E− | 100 | KY415661.1 |
| (A/duck/Hubei/ZYSYF2/2016(H5N1)) segment | | | | 06 | | |
| 7 matrix protein 2 (M2) and matrix protein 1 | | | | | | |
| (M1) genes, complete cds | | | | | | |
| Influenza A virus | 52.8 | 52.8 | 40% | 5.00E− | 100 | KY415660.1 |
| (A/chicken/Hubei/ZYSJF15/2016(H9N2)) | | | | 06 | | |
| segment 7 matrix protein 2 (M2) and matrix | | | | | | |
| protein 1 (M1) genes, complete cds | | | | | | |
| Influenza A virus (A/Fujian/S03/2015(H7N9)) | 52.8 | 52.8 | 40% | 5.00E− | 100 | KY286430.1 |
| segment 7 matrix protein 2 (M2) and matrix | | | | 06 | | |
| protein 1 (M1) genes, complete cds | | | | | | |
| Influenza A virus | 52.8 | 52.8 | 40% | 5.00E− | 100 | KY286422.1 |
| (A/Minhang/S01/2013(H7N9)) segment 7 | | | | 06 | | |
| matrix protein 2 (M2) and matrix protein 1 (M1) | | | | | | |
| genes, complete cds | | | | | | |
| Influenza A virus (A/lion/Hubei/1- | 52.8 | 52.8 | 40% | 5.00E− | 100 | KX571057.1 |
| 2/2016(H5N1)) segment 7 matrix protein 2 | | | | 06 | | |
| (M2) and matrix protein 1 (M1) genes, complete | | | | | | |
| cds | | | | | | |
| Influenza A virus (A/Huai'an/065/2014(H7N9)) | 52.8 | 52.8 | 40% | 5.00E− | 100 | KX215927.1 |
| segment 7 matrix protein 2 (M2) and matrix | | | | 06 | | |
| protein 1 (M1) genes, complete cds | | | | | | |
| Influenza A virus (A/Huai'an/002/2015(H7N9)) | 52.8 | 52.8 | 40% | 5.00E− | 100 | KX215925.1 |
| segment 7 matrix protein 2 (M2) and matrix | | | | 06 | | |
| protein 1 (M1) genes, complete cds | | | | | | |
| Influenza A virus (A/Huai'an/001/2015(H7N9)) | 52.8 | 52.8 | 40% | 5.00E− | 100 | KX215924.1 |
| segment 7 matrix protein 2 (M2) and matrix | | | | 06 | | |
| protein 1 (M1) genes, complete cds | | | | | | |
| Influenza A virus | 52.8 | 52.8 | 40% | 5.00E− | 100 | KX215923.1 |
| (A/duck/Huai'an/053/2014(H7N9)) segment 7 | | | | 06 | | |
| matrix protein 2 (M2) and matrix protein 1 (M1) | | | | | | |
| genes, complete cds | | | | | | |

In some embodiments, the composition described herein comprises probe 6. In additional embodiments, probe 6 may hybridize to a target nucleotide molecule that is a part of Influenza B virus, causing Influenza B. In some embodiments, probe 6 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 6. In some embodiments, probe 6 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 6. Table 2 shows potential target nucleotide molecules in the Influenza B virus.

TABLE 2
Potential target nucleotide molecules in the Influenza B virus

| | Max | Total | Query | E | Per. | |
|---|---|---|---|---|---|---|
| Description | Score | Score | Cover | value | ident | Accession |
| Influenza B virus (B/Maryland/363_1/2013) | 34.2 | 34.2 | 100% | 0.093 | 100 | MT270093.1 |
| segment 8 nuclear export protein (NEP) and | | | | | | |
| nonstructural protein 1 (NS1) genes, complete | | | | | | |
| cds | | | | | | |
| Influenza B virus (B/Maryland/142_1/2013) | 34.2 | 34.2 | 100% | 0.093 | 100 | MT270033.1 |
| segment 8 nuclear export protein (NEP) and | | | | | | |
| nonstructural protein 1 (NS1) genes, complete | | | | | | |
| cds | | | | | | |
| Influenza B virus (B/Maryland/326_1/2013) | 34.2 | 34.2 | 100% | 0.093 | 100 | MT270025.1 |
| segment 8 nuclear export protein (NEP) and | | | | | | |
| nonstructural protein 1 (NS1) genes, complete | | | | | | |
| cds | | | | | | |
| Influenza B virus (B/Maryland/321_1/2013) | 34.2 | 34.2 | 100% | 0.093 | 100 | MT270018.1 |
| segment 8 nuclear export protein (NEP) and | | | | | | |
| nonstructural protein 1 (NS1) genes, complete | | | | | | |
| cds | | | | | | |
| Influenza B virus (B/Maryland/342_1/2013) | 34.2 | 34.2 | 100% | 0.093 | 100 | MT269986.1 |
| segment 8 nuclear export protein (NEP) and | | | | | | |
| nonstructural protein 1 (NS1) genes, complete | | | | | | |
| cds | | | | | | |
| Influenza B virus (B/Maryland/298_1/2013) | 34.2 | 34.2 | 100% | 0.093 | 100 | MT269978.1 |
| segment 8 nuclear export protein (NEP) and | | | | | | |
| nonstructural protein 1 (NS1) genes, complete | | | | | | |
| cds | | | | | | |
| Influenza B virus (B/Maryland/119_1/2013) | 34.2 | 34.2 | 100% | 0.093 | 100 | MT269962.1 |
| segment 8 nuclear export protein (NEP) and | | | | | | |
| nonstructural protein 1 (NS1) genes, complete | | | | | | |
| cds | | | | | | |
| Influenza B virus (B/Maryland/255_1/2013) | 34.2 | 34.2 | 100% | 0.093 | 100 | MT269948.1 |
| segment 8 nuclear export protein (NEP) and | | | | | | |
| nonstructural protein 1 (NS1) genes, complete | | | | | | |
| cds | | | | | | |
| Influenza B virus (B/Virginia/03/2020) segment | 34.2 | 34.2 | 100% | 0.093 | 100 | MT243871.1 |
| 8 nuclear export protein (NEP) and | | | | | | |
| nonstructural protein 1 (NS1) genes, complete | | | | | | |
| cds | | | | | | |
| Influenza B virus (B/Utah/05/2020) segment 8 | 34.2 | 34.2 | 100% | 0.093 | 100 | MT243839.1 |
| nuclear export protein (NEP) and nonstructural | | | | | | |
| protein 1 (NS1) genes, complete cds | | | | | | |
| Influenza B virus (B/Utah/04/2020) segment 8 | 34.2 | 34.2 | 100% | 0.093 | 100 | MT243831.1 |
| nuclear export protein (NEP) and nonstructural | | | | | | |
| protein 1 (NS1) genes, complete cds | | | | | | |
| Influenza B virus (B/Texas/152/2019) segment | 34.2 | 34.2 | 100% | 0.093 | 100 | MT243799.1 |
| 8 nuclear export protein (NEP) and | | | | | | |
| nonstructural protein 1 (NS1) genes, complete | | | | | | |
| cds | | | | | | |
| Influenza B virus (B/Maine/03/2020) segment 8 | 34.2 | 34.2 | 100% | 0.093 | 100 | MT243199.1 |
| nuclear export protein (NEP) and nonstructural | | | | | | |
| protein 1 (NS1) genes, complete cds | | | | | | |
| Influenza B virus (B/Iowa/04/2020) segment 8 | 34.2 | 34.2 | 100% | 0.093 | 100 | MT243143.1 |
| nuclear export protein (NEP) and nonstructural | | | | | | |
| protein 1 (NS1) genes, complete cds | | | | | | |
| Influenza B virus (B/Delaware/04/2020) | 34.2 | 34.2 | 100% | 0.093 | 100 | MT242871.1 |
| segment 8 nuclear export protein (NEP) and | | | | | | |
| nonstructural protein 1 (NS1) genes, complete | | | | | | |
| cds | | | | | | |
| Influenza B virus (B/California/13/2020) | 34.2 | 34.2 | 100% | 0.093 | 100 | MT242783.1 |
| segment 8 nuclear export protein (NEP) and | | | | | | |
| nonstructural protein 1 (NS1) genes, complete | | | | | | |
| cds | | | | | | |
| Influenza B virus (B/District Of | 34.2 | 34.2 | 100% | 0.093 | 100 | MT242663.1 |
| Columbia/32/2019) segment 8 nuclear export | | | | | | |
| protein (NEP) and nonstructural protein 1 | | | | | | |
| (NS1) genes, complete cds | | | | | | |
| Influenza B virus (B/Virginia/01/2020) segment | 34.2 | 34.2 | 100% | 0.093 | 100 | MT242637.1 |
| 8 nuclear export protein (NEP) and | | | | | | |
| nonstructural protein 1 (NS1) genes, complete | | | | | | |
| cds | | | | | | |
| Influenza B virus (B/Virginia/29/2019) segment | 34.2 | 34.2 | 100% | 0.093 | 100 | MT105800.1 |
| 8 nuclear export protein (NEP) and | | | | | | |
| nonstructural protein 1 (NS1) genes, complete | | | | | | |
| cds | | | | | | |
| Influenza B virus (B/Virginia/28/2019) segment | 34.2 | 34.2 | 100% | 0.093 | 100 | MT105796.1 |
| 8 nuclear export protein (NEP) and | | | | | | |
| nonstructural protein 1 (NS1) genes, complete | | | | | | |
| cds | | | | | | |
| Influenza B virus (B/Connecticut/12/2019) | 34.2 | 34.2 | 100% | 0.093 | 100 | MT105580.1 |
| segment 8 nuclear export protein (NEP) and | | | | | | |
| nonstructural protein 1 (NS1) genes, complete | | | | | | |
| cds | | | | | | |
| Influenza B virus (B/Wisconsin/49/2019) | 34.2 | 34.2 | 100% | 0.093 | 100 | MT057825.1 |
| segment 8 nuclear export protein (NEP) and | | | | | | |
| nonstructural protein 1 (NS1) genes, complete | | | | | | |
| cds | | | | | | |
| Influenza B virus (B/Washington/28/2019) | 34.2 | 34.2 | 100% | 0.093 | 100 | MT057791.1 |
| segment 8 nuclear export protein (NEP) and | | | | | | |
| nonstructural protein 1 (NS1) genes, complete | | | | | | |
| cds | | | | | | |
| Influenza B virus (B/Oregon/12/2019) segment | 34.2 | 34.2 | 100% | 0.093 | 100 | MT057567.1 |
| 8 nuclear export protein (NEP) and | | | | | | |
| nonstructural protein 1 (NS1) genes, complete | | | | | | |
| cds | | | | | | |
| Influenza B virus (B/New Jersey/15/2019) | 34.2 | 34.2 | 100% | 0.093 | 100 | MT057415.1 |
| segment 8 nuclear export protein (NEP) and | | | | | | |
| nonstructural protein 1 (NS1) genes, complete | | | | | | |
| cds | | | | | | |
| Influenza B virus (B/New Hampshire/19/2019) | 34.2 | 34.2 | 100% | 0.093 | 100 | MT057399.1 |
| segment 8 nuclear export protein (NEP) and | | | | | | |
| nonstructural protein 1 (NS1) genes, complete | | | | | | |
| cds | | | | | | |
| Influenza B virus (B/Minnesota/29/2019) | 34.2 | 34.2 | 100% | 0.093 | 100 | MT057143.1 |
| segment 8 nuclear export protein (NEP) and | | | | | | |
| nonstructural protein 1 (NS1) genes, complete | | | | | | |
| cds | | | | | | |
| Influenza B virus (B/Georgia/09/2019) segment | 34.2 | 34.2 | 100% | 0.093 | 100 | MT056951.1 |
| 8 nuclear export protein (NEP) and | | | | | | |
| nonstructural protein 1 (NS1) genes, complete | | | | | | |
| cds | | | | | | |
| Influenza B virus (B/California/88/2019) | 34.2 | 34.2 | 100% | 0.093 | 100 | MT056747.1 |
| segment 8 nuclear export protein (NEP) and | | | | | | |
| nonstructural protein 1 (NS1) genes, complete | | | | | | |
| cds | | | | | | |
| Influenza B virus (B/Maine/20/2019) segment 8 | 34.2 | 34.2 | 100% | 0.093 | 100 | MT056647.1 |
| nuclear export protein (NEP) and nonstructural | | | | | | |
| protein 1 (NS1) genes, complete cds | | | | | | |
| Influenza B virus (B/Georgia/08/2019) segment | 34.2 | 34.2 | 100% | 0.093 | 100 | MT056628.1 |
| 8 nuclear export protein (NEP) and | | | | | | |
| nonstructural protein 1 (NS1) genes, complete | | | | | | |
| cds | | | | | | |
| Influenza B virus (B/Massachusetts/2/2012 BX- | 34.2 | 34.2 | 100% | 0.093 | 100 | MT056025.1 |
| 51C) segment 8 nuclear export protein (NEP) | | | | | | |
| and nonstructural protein 1 (NS1) genes, | | | | | | |
| complete cds | | | | | | |
| Influenza B virus (B/Wisconsin/41/2019) | 34.2 | 34.2 | 100% | 0.093 | 100 | MT049900.1 |
| segment 8 nuclear export protein (NEP) and | | | | | | |
| nonstructural protein 1 (NS1) genes, complete | | | | | | |
| cds | | | | | | |
| Influenza B virus (B/Texas/43/2019) segment 8 | 34.2 | 34.2 | 100% | 0.093 | 100 | MT049749.1 |
| nuclear export protein (NEP) and nonstructural | | | | | | |
| protein 1 (NS1) genes, complete cds | | | | | | |
| Influenza B virus (B/New Jersey/15/2019) | 34.2 | 34.2 | 100% | 0.093 | 100 | MT049557.1 |
| segment 8 nuclear export protein (NEP) and | | | | | | |
| nonstructural protein 1 (NS1) genes, complete | | | | | | |
| cds | | | | | | |
| Influenza B virus (B/Florida/72/2019) segment | 34.2 | 34.2 | 100% | 0.093 | 100 | MT049157.1 |
| 8 nuclear export protein (NEP) and | | | | | | |
| nonstructural protein 1 (NS1) genes, complete | | | | | | |
| cds | | | | | | |
| Influenza B virus (B/shanghai/114/2018) | 34.2 | 34.2 | 100% | 0.093 | 100 | MT040799.1 |
| segment 8 nuclear export protein (NEP) and | | | | | | |
| nonstructural protein 1 (NS1) genes, complete | | | | | | |
| cds | | | | | | |
| Influenza B virus (B/Wisconsin/43/2019) | 34.2 | 34.2 | 100% | 0.093 | 100 | MT030768.1 |
| segment 8 nuclear export protein (NEP) and | | | | | | |
| nonstructural protein 1 (NS1) genes, complete | | | | | | |
| cds | | | | | | |
| Influenza B virus (B/Virginia/26/2019) segment | 34.2 | 34.2 | 100% | 0.093 | 100 | MT030720.1 |
| 8 nuclear export protein (NEP) and | | | | | | |
| nonstructural protein 1 (NS1) genes, complete | | | | | | |
| cds | | | | | | |
| Influenza B virus (B/Minnesota/26/2019) | 34.2 | 34.2 | 100% | 0.093 | 100 | MT030145.1 |
| segment 8 nuclear export protein (NEP) and | | | | | | |
| nonstructural protein 1 (NS1) genes, complete | | | | | | |
| cds | | | | | | |
| Influenza B virus (B/Iowa/33/2019) segment 8 | 34.2 | 34.2 | 100% | 0.093 | 100 | MT029882.1 |
| nuclear export protein (NEP) and nonstructural | | | | | | |
| protein 1 (NS1) genes, complete cds | | | | | | |
| Influenza B virus (B/Idaho/19/2019) segment 8 | 34.2 | 34.2 | 100% | 0.093 | 100 | MT029778.1 |
| nuclear export protein (NEP) and nonstructural | | | | | | |
| protein 1 (NS1) genes, complete cds | | | | | | |
| Influenza B virus (B/Hawaii/49/2019) segment | 34.2 | 34.2 | 100% | 0.093 | 100 | MT029754.1 |
| 8 nuclear export protein (NEP) and | | | | | | |
| nonstructural protein 1 (NS1) genes, complete | | | | | | |
| cds | | | | | | |
| Influenza B virus (B/Florida/78/2019) segment | 34.2 | 34.2 | 100% | 0.093 | 100 | MT029706.1 |
| 8 nuclear export protein (NEP) and | | | | | | |
| nonstructural protein 1 (NS1) genes, complete | | | | | | |
| cds | | | | | | |
| Influenza B virus (B/California/79/2019) | 34.2 | 34.2 | 100% | 0.093 | 100 | MT029530.1 |
| segment 8 nuclear export protein (NEP) and | | | | | | |
| nonstructural protein 1 (NS1) genes, complete | | | | | | |
| cds | | | | | | |
| Influenza B virus (B/California/77/2019) | 34.2 | 34.2 | 100% | 0.093 | 100 | MT029522.1 |
| segment 8 nuclear export protein (NEP) and | | | | | | |
| nonstructural protein 1 (NS1) genes, complete | | | | | | |
| cds | | | | | | |
| Influenza B virus (B/Arkansas/10/2019) | 34.2 | 34.2 | 100% | 0.093 | 100 | MT029490.1 |
| segment 8 nuclear export protein (NEP) and | | | | | | |
| nonstructural protein 1 (NS1) genes, complete | | | | | | |
| cds | | | | | | |
| Influenza B virus (B/Alabama/12/2019) | 34.2 | 34.2 | 100% | 0.093 | 100 | MT029394.1 |
| segment 8 nuclear export protein (NEP) and | | | | | | |
| nonstructural protein 1 (NS1) genes, complete | | | | | | |
| cds | | | | | | |
| Influenza B virus (B/Wisconsin/36/2019) | 34.2 | 34.2 | 100% | 0.093 | 100 | MN950409.1 |
| segment 8 nuclear export protein (NEP) and | | | | | | |
| nonstructural protein 1 (NS1) genes, complete | | | | | | |
| cds | | | | | | |
| Influenza B virus (B/Texas/40/2019) segment 8 | 34.2 | 34.2 | 100% | 0.093 | 100 | MN950354.1 |
| nuclear export protein (NEP) and nonstructural | | | | | | |
| protein 1 (NS1) genes, complete cds | | | | | | |
| Influenza B virus (B/North Carolina/10/2019) | 34.2 | 34.2 | 100% | 0.093 | 100 | MN950257.1 |
| segment 8 nuclear export protein (NEP) and | | | | | | |
| nonstructural protein 1 (NS1) genes, complete | | | | | | |
| cds | | | | | | |
| Influenza B virus (B/North Carolina/09/2019) | 34.2 | 34.2 | 100% | 0.093 | 100 | MN950249.1 |
| segment 8 nuclear export protein (NEP) and | | | | | | |
| nonstructural protein 1 (NS1) genes, complete | | | | | | |
| cds | | | | | | |
| Influenza B virus (B/New York/14/2019) | 34.2 | 34.2 | 100% | 0.093 | 100 | MN950241.1 |
| segment 8 nuclear export protein (NEP) and | | | | | | |
| nonstructural protein 1 (NS1) genes, complete | | | | | | |
| cds | | | | | | |
| Influenza B virus (B/New Jersey/10/2019) | 34.2 | 34.2 | 100% | 0.093 | 100 | MN950217.1 |
| segment 8 nuclear export protein (NEP) and | | | | | | |
| nonstructural protein 1 (NS1) genes, complete | | | | | | |
| cds | | | | | | |
| Influenza B virus (B/Massachusetts/18/2019) | 34.2 | 34.2 | 100% | 0.093 | 100 | MN950113.1 |
| segment 8 nuclear export protein (NEP) and | | | | | | |
| nonstructural protein 1 (NS1) genes, complete | | | | | | |
| cds | | | | | | |
| Influenza B virus (B/Maryland/25/2019) | 34.2 | 34.2 | 100% | 0.093 | 100 | MN950097.1 |
| segment 8 nuclear export protein (NEP) and | | | | | | |
| nonstructural protein 1 (NS1) genes, complete | | | | | | |
| cds | | | | | | |
| Influenza B virus (B/Florida/67/2019) segment | 34.2 | 34.2 | 100% | 0.093 | 100 | MN949353.1 |
| 8 nuclear export protein (NEP) and | | | | | | |
| nonstructural protein 1 (NS1) genes, complete | | | | | | |
| cds | | | | | | |
| Influenza B virus (B/Florida/66/2019) segment | 34.2 | 34.2 | 100% | 0.093 | 100 | MN949345.1 |
| 8 nuclear export protein (NEP) and | | | | | | |
| nonstructural protein 1 (NS1) genes, complete | | | | | | |
| cds | | | | | | |
| Influenza B virus (B/Florida/64/2019) segment | 34.2 | 34.2 | 100% | 0.093 | 100 | MN949128.1 |
| 8 nuclear export protein (NEP) and | | | | | | |
| nonstructural protein 1 (NS1) genes, complete | | | | | | |
| cds | | | | | | |
| Influenza B virus (B/Wisconsin/34/2019) | 34.2 | 34.2 | 100% | 0.093 | 100 | MN881765.1 |
| segment 8 nuclear export protein (NEP) and | | | | | | |
| nonstructural protein 1 (NS1) genes, complete | | | | | | |
| cds | | | | | | |
| Influenza B virus (B/Wisconsin/33/2019) | 34.2 | 34.2 | 100% | 0.093 | 100 | MN881757.1 |
| segment 8 nuclear export protein (NEP) and | | | | | | |
| nonstructural protein 1 (NS1) genes, complete | | | | | | |
| cds | | | | | | |
| Influenza B virus (B/South Dakota/12/2019) | 34.2 | 34.2 | 100% | 0.093 | 100 | MN881701.1 |
| segment 8 nuclear export protein (NEP) and | | | | | | |
| nonstructural protein 1 (NS1) genes, complete | | | | | | |
| cds | | | | | | |
| Influenza B virus (B/California/51/2019) | 34.2 | 34.2 | 100% | 0.093 | 100 | MN881509.1 |
| segment 8 nuclear export protein (NEP) and | | | | | | |
| nonstructural protein 1 (NS1) genes, complete | | | | | | |
| cds | | | | | | |
| Influenza B virus (B/Arizona/23/2019) segment | 34.2 | 34.2 | 100% | 0.093 | 100 | MN881445.1 |
| 8 nuclear export protein (NEP) and | | | | | | |
| nonstructural protein 1 (NS1) genes, complete | | | | | | |
| cds | | | | | | |
| Influenza B virus (B/Alabama/10/2019) | 34.2 | 34.2 | 100% | 0.093 | 100 | MN881437.1 |
| segment 8 nuclear export protein (NEP) and | | | | | | |
| nonstructural protein 1 (NS1) genes, complete | | | | | | |
| cds | | | | | | |
| Influenza B virus | 34.2 | 34.2 | 100% | 0.093 | 100 | MN874046.1 |
| (B/Yokosuka/NHRC_OID_FDX70676/2019) | | | | | | |
| segment 8 nuclear export protein (NEP) and | | | | | | |
| nonstructural protein 1 (NS1) genes, complete | | | | | | |
| cds | | | | | | |
| Influenza B virus | 34.2 | 34.2 | 100% | 0.093 | 100 | MN874029.1 |
| (B/California/NHRC_OID_SAR42258N/2019) | | | | | | |
| segment 8 nuclear export protein (NEP) and | | | | | | |
| nonstructural protein 1 (NS1) genes, complete | | | | | | |
| cds | | | | | | |
| Influenza B virus | 34.2 | 34.2 | 100% | 0.093 | 100 | MN874024.1 |
| (B/California/NHRC_OID_BRD81375N/2019) | | | | | | |
| segment 8 nuclear export protein (NEP) and | | | | | | |
| nonstructural protein 1 (NS1) genes, complete | | | | | | |
| cds | | | | | | |
| Influenza B virus | 34.2 | 34.2 | 100% | 0.093 | 100 | MN874006.1 |
| (B/California/NHRC_OID_FDX12346/2019) | | | | | | |
| segment 8 nuclear export protein (NEP) and | | | | | | |
| nonstructural protein 1 (NS1) genes, complete | | | | | | |
| cds | | | | | | |
| Influenza B virus (B/Gyeongbuk/923/2018) | 34.2 | 34.2 | 100% | 0.093 | 100 | MN822675.1 |
| segment 8 nuclear export protein (NEP) and | | | | | | |
| nonstructural protein 1 (NS1) genes, complete | | | | | | |
| cds | | | | | | |
| Influenza B virus (B/Wyoming/7116/2018) | 34.2 | 34.2 | 100% | 0.093 | 100 | MK380634.1 |
| segment 8 nuclear export protein (NEP) and | | | | | | |
| nonstructural protein 1 (NS1) genes, complete | | | | | | |
| cds | | | | | | |
| Influenza B virus (B/Texas/7112/2018) segment | 34.2 | 34.2 | 100% | 0.093 | 100 | MK380630.1 |
| 8 nuclear export protein (NEP) and | | | | | | |
| nonstructural protein 1 (NS1) genes, complete | | | | | | |
| cds | | | | | | |
| Influenza B virus (B/Texas/7111/2018) segment | 34.2 | 34.2 | 100% | 0.093 | 100 | MK380629.1 |
| 8 nuclear export protein (NEP) and | | | | | | |
| nonstructural protein 1 (NS1) genes, complete | | | | | | |
| cds | | | | | | |
| Influenza B virus (B/Ohio/7108/2018) segment | 34.2 | 34.2 | 100% | 0.093 | 100 | MK380626.1 |
| 8 nuclear export protein (NEP) and | | | | | | |
| nonstructural protein 1 (NS1) genes, complete | | | | | | |
| cds | | | | | | |
| Influenza B virus (B/Ohio/7107/2018) segment | 34.2 | 34.2 | 100% | 0.093 | 100 | MK380625.1 |
| 8 nuclear export protein (NEP) and | | | | | | |
| nonstructural protein 1 (NS1) genes, complete | | | | | | |
| cds | | | | | | |
| Influenza B virus (B/New Jersey/7104/2018) | 34.2 | 34.2 | 100% | 0.093 | 100 | MK380622.1 |
| segment 8 nuclear export protein (NEP) and | | | | | | |
| nonstructural protein 1 (NS1) genes, complete | | | | | | |
| cds | | | | | | |
| Influenza B virus (B/New Jersey/7102/2018) | 34.2 | 34.2 | 100% | 0.093 | 100 | MK380620.1 |
| segment 8 nuclear export protein (NEP) and | | | | | | |
| nonstructural protein 1 (NS1) genes, complete | | | | | | |
| cds | | | | | | |
| Influenza B virus (B/New Jersey/7101/2018) | 34.2 | 34.2 | 100% | 0.093 | 100 | MK380619.1 |
| segment 8 nuclear export protein (NEP) and | | | | | | |
| nonstructural protein 1 (NS1) genes, complete | | | | | | |
| cds | | | | | | |
| Influenza B virus (B/Kansas/7097/2018) | 34.2 | 34.2 | 100% | 0.093 | 100 | MK380615.1 |
| segment 8 nuclear export protein (NEP) and | | | | | | |
| nonstructural protein 1 (NS1) genes, complete | | | | | | |
| cds | | | | | | |
| Influenza B virus (B/Japan/7095/2018) segment | 34.2 | 34.2 | 100% | 0.093 | 100 | MK380613.1 |
| 8 nuclear export protein (NEP) and | | | | | | |
| nonstructural protein 1 (NS1) genes, complete | | | | | | |
| cds | | | | | | |
| Influenza B virus (B/Illinois/7094/2018) | 34.2 | 34.2 | 100% | 0.093 | 100 | MK380612.1 |
| segment 8 nuclear export protein (NEP) and | | | | | | |
| nonstructural protein 1 (NS1) genes, complete | | | | | | |
| cds | | | | | | |
| Influenza B virus (B/Idaho/7093/2018) segment | 34.2 | 34.2 | 100% | 0.093 | 100 | MK380611.1 |
| 8 nuclear export protein (NEP) and | | | | | | |
| nonstructural protein 1 (NS1) genes, complete | | | | | | |
| cds | | | | | | |
| Influenza B virus (B/Georgia/7092/2018) | 34.2 | 34.2 | 100% | 0.093 | 100 | MK380610.1 |
| segment 8 nuclear export protein (NEP) and | | | | | | |
| nonstructural protein 1 (NS1) genes, complete | | | | | | |
| cds | | | | | | |
| Influenza B virus (B/Florida/7091/2018) | 34.2 | 34.2 | 100% | 0.093 | 100 | MK380609.1 |
| segment 8 nuclear export protein (NEP) and | | | | | | |
| nonstructural protein 1 (NS1) genes, complete | | | | | | |
| cds | | | | | | |
| Influenza B virus (B/Arizona/7089/2018) | 34.2 | 34.2 | 100% | 0.093 | 100 | MK380607.1 |
| segment 8 nuclear export protein (NEP) and | | | | | | |
| nonstructural protein 1 (NS1) genes, complete | | | | | | |
| cds | | | | | | |
| Influenza B virus (B/Alabama/7088/2018) | 34.2 | 34.2 | 100% | 0.093 | 100 | MK380606.1 |
| segment 8 nuclear export protein (NEP) and | | | | | | |
| nonstructural protein 1 (NS1) genes, complete | | | | | | |
| cds | | | | | | |
| Influenza B virus (B/Virginia/7086/2018) | 34.2 | 34.2 | 100% | 0.093 | 100 | MK380387.1 |
| segment 8 nuclear export protein (NEP) and | | | | | | |
| nonstructural protein 1 (NS1) genes, complete | | | | | | |
| cds | | | | | | |
| Influenza B virus (B/Virginia/7085/2018) | 34.2 | 34.2 | 100% | 0.093 | 100 | MK380386.1 |
| segment 8 nuclear export protein (NEP) and | | | | | | |
| nonstructural protein 1 (NS1) genes, complete | | | | | | |
| cds | | | | | | |
| Influenza B virus (B/Virginia/7084/2018) | 34.2 | 34.2 | 100% | 0.093 | 100 | MK380385.1 |
| segment 8 nuclear export protein (NEP) and | | | | | | |
| nonstructural protein 1 (NS1) genes, complete | | | | | | |
| cds | | | | | | |
| Influenza B virus (B/Texas/7083/2018) segment | 34.2 | 34.2 | 100% | 0.093 | 100 | MK380384.1 |
| 8 nuclear export protein (NEP) and | | | | | | |
| nonstructural protein 1 (NS1) genes, complete | | | | | | |
| cds | | | | | | |
| Influenza B virus (B/Texas/7082/2018) segment | 34.2 | 34.2 | 100% | 0.093 | 100 | MK380383.1 |
| 8 nuclear export protein (NEP) and | | | | | | |
| nonstructural protein 1 (NS1) genes, complete | | | | | | |
| cds | | | | | | |
| Influenza B virus (B/Texas/7081/2018) segment | 34.2 | 34.2 | 100% | 0.093 | 100 | MK380382.1 |
| 8 nuclear export protein (NEP) and | | | | | | |
| nonstructural protein 1 (NS1) genes, complete | | | | | | |
| cds | | | | | | |
| Influenza B virus (B/Texas/7080/2018) segment | 34.2 | 34.2 | 100% | 0.093 | 100 | MK380381.1 |
| 8 nuclear export protein (NEP) and | | | | | | |
| nonstructural protein 1 (NS1) genes, complete | | | | | | |
| cds | | | | | | |
| Influenza B virus (B/Texas/7079/2018) segment | 34.2 | 34.2 | 100% | 0.093 | 100 | MK380380.1 |
| 8 nuclear export protein (NEP) and | | | | | | |
| nonstructural protein 1 (NS1) genes, complete | | | | | | |
| cds | | | | | | |
| Influenza B virus (B/Texas/7078/2018) segment | 34.2 | 34.2 | 100% | 0.093 | 100 | MK380379.1 |
| 8 nuclear export protein (NEP) and | | | | | | |
| nonstructural protein 1 (NS1) genes, complete | | | | | | |
| cds | | | | | | |
| Influenza B virus (B/Texas/7077/2018) segment | 34.2 | 34.2 | 100% | 0.093 | 100 | MK380378.1 |
| 8 nuclear export protein (NEP) and | | | | | | |
| nonstructural protein 1 (NS1) genes, complete | | | | | | |
| cds | | | | | | |
| Influenza B virus (B/Texas/7076/2018) segment | 34.2 | 34.2 | 100% | 0.093 | 100 | MK380377.1 |
| 8 nuclear export protein (NEP) and | | | | | | |
| nonstructural protein 1 (NS1) genes, complete | | | | | | |
| cds | | | | | | |
| Influenza B virus (B/Texas/7075/2018) segment | 34.2 | 34.2 | 100% | 0.093 | 100 | MK380376.1 |
| 8 nuclear export protein (NEP) and | | | | | | |
| nonstructural protein 1 (NS1) genes, complete | | | | | | |
| cds | | | | | | |
| Influenza B virus (B/Spain/7074/2018) segment | 34.2 | 34.2 | 100% | 0.093 | 100 | MK380375.1 |
| 8 nuclear export protein (NEP) and | | | | | | |
| nonstructural protein 1 (NS1) genes, complete | | | | | | |
| cds | | | | | | |
| Influenza B virus (B/South Korea/7073/2018) | 34.2 | 34.2 | 100% | 0.093 | 100 | MK380374.1 |
| segment 8 nuclear export protein (NEP) and | | | | | | |
| nonstructural protein 1 (NS1) genes, complete | | | | | | |
| cds | | | | | | |
| Influenza B virus (B/Maryland/363_1/2013) | 37.4 | 37.4 | 100% | 0.02 | 95 | MT270093.1 |
| segment 8 nuclear export protein (NEP) and | | | | | | |
| nonstructural protein 1 (NS1) genes, complete | | | | | | |
| cds | | | | | | |
| Influenza B virus (B/Utah/04/2020) segment 8 | 37.4 | 37.4 | 100% | 0.02 | 95 | MT243831.1 |
| nuclear export protein (NEP) and nonstructural | | | | | | |
| protein 1 (NS1) genes, complete cds | | | | | | |
| Influenza B virus (B/Massachusetts/2/2012 BX- | 37.4 | 37.4 | 100% | 0.02 | 95 | MT056025.1 |
| 51C) segment 8 nuclear export protein (NEP) | | | | | | |
| and nonstructural protein 1 (NS1) genes, | | | | | | |
| complete cds | | | | | | |
| Influenza B virus (B/Hawaii/46/2019) segment | 37.4 | 37.4 | 100% | 0.02 | 95 | MT049181.1 |
| 8 nuclear export protein (NEP) and | | | | | | |
| nonstructural protein 1 (NS1) genes, complete | | | | | | |
| cds | | | | | | |
| Influenza B virus (B/Georgia/06/2019) segment | 37.4 | 37.4 | 100% | 0.02 | 95 | MT029722.1 |
| 8 nuclear export protein (NEP) and | | | | | | |
| nonstructural protein 1 (NS1) genes, complete | | | | | | |
| cds | | | | | | |
| Influenza B virus (B/Arizona/25/2019) segment | 37.4 | 37.4 | 100% | 0.02 | 95 | MN881461.1 |
| 8 nuclear export protein (NEP) and | | | | | | |
| nonstructural protein 1 (NS1) genes, complete | | | | | | |
| cds | | | | | | |
| Influenza B virus (B/Hawaii/43/2019) segment | 37.4 | 37.4 | 100% | 0.02 | 95 | MN819387.1 |
| 8 nuclear export protein (NEP) and | | | | | | |
| nonstructural protein 1 (NS1) genes, complete | | | | | | |
| cds | | | | | | |
| Influenza B virus (B/Hawaii/39/2019) segment | 37.4 | 37.4 | 100% | 0.02 | 95 | MN754109.1 |
| 8 nuclear export protein (NEP) and | | | | | | |
| nonstructural protein 1 (NS1) genes, complete | | | | | | |
| cds | | | | | | |
| Influenza B virus | 37.4 | 37.4 | 100% | 0.02 | 95 | MN589098.1 |
| (B/Singapore/DMS2059/2012) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN589002.1 |
| (B/Singapore/DMS1978/2012) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588922.1 |
| (B/Singapore/DMS1889/2012) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588906.1 |
| (B/Singapore/DMS1883/2012) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588874.1 |
| (B/Singapore/DMS1849/2012) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588850.1 |
| (B/Singapore/DMS1587/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588842.1 |
| (B/Singapore/DMS1577/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588834.1 |
| (B/Singapore/DMS1566/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588818.1 |
| (B/Singapore/DMS1559/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588810.1 |
| (B/Singapore/DMS1557/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588802.1 |
| (B/Singapore/DMS1555/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588794.1 |
| (B/Singapore/DMS1532/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588786.1 |
| (B/Singapore/DMS1514/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588778.1 |
| (B/Singapore/DMS1483/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588741.1 |
| (B/Singapore/DMS1625/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588738.1 |
| (B/Singapore/DMS1617/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588737.1 |
| (B/Singapore/DMS1615/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588736.1 |
| (B/Singapore/DMS1613/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588735.1 |
| (B/Singapore/DMS1606/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588734.1 |
| (B/Singapore/DMS1604/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588733.1 |
| (B/Singapore/DMS1588/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588732.1 |
| (B/Singapore/DMS1579/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588731.1 |
| (B/Singapore/DMS1576/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588730.1 |
| (B/Singapore/DMS1573/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588729.1 |
| (B/Singapore/DMS1569/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588728.1 |
| (B/Singapore/DMS1565/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588727.1 |
| (B/Singapore/DMS1562/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588726.1 |
| (B/Singapore/DMS1551/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588725.1 |
| (B/Singapore/DMS1548/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588724.1 |
| (B/Singapore/DMS1546/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588723.1 |
| (B/Singapore/DMS1545/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588722.1 |
| (B/Singapore/DMS1542/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588721.1 |
| (B/Singapore/DMS1534/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588720.1 |
| (B/Singapore/DMS1530/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588719.1 |
| (B/Singapore/DMS1529/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588718.1 |
| (B/Singapore/DMS1527/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588717.1 |
| (B/Singapore/DMS1508/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588716.1 |
| (B/Singapore/DMS1503/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588715.1 |
| (B/Singapore/DMS1499/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588714.1 |
| (B/Singapore/DMS1498/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588713.1 |
| (B/Singapore/DMS1497/2011) segment 8 | | | | | | |
| nuclear export protein (NEP) gene, partial cds; | | | | | | |
| and nonstructural protein 1 (NS1) gene, | | | | | | |
| complete cds | | | | | | |
| Influenza B virus | 37.4 | 37.4 | 100% | 0.02 | 95 | MN588712.1 |
| (B/Singapore/DMS1495/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588711.1 |
| (B/Singapore/DMS1493/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588710.1 |
| (B/Singapore/DMS1489/2011) segment 8 | | | | | | |
| nuclear export protein (NEP) and nonstructural | | | | | | |
| protein 1 (NS1) genes, partial cds | | | | | | |
| Influenza B virus | 37.4 | 37.4 | 100% | 0.02 | 95 | MN588709.1 |
| (B/Singapore/DMS1488/2011) segment 8 | | | | | | |
| nuclear export protein (NEP) and nonstructural | | | | | | |
| protein 1 (NS1) genes, complete cds | | | | | | |
| Influenza B virus | 37.4 | 37.4 | 100% | 0.02 | 95 | MN588708.1 |
| (B/Singapore/DMS1486/2011) segment 8 | | | | | | |
| nuclear export protein (NEP) and nonstructural | | | | | | |
| protein 1 (NS1) genes, partial cds | | | | | | |
| Influenza B virus (B/Singapore/TTSH-69/2012) | 37.4 | 37.4 | 100% | 0.02 | 95 | MN396694.1 |
| segment 8 nuclear export protein (NEP) gene, | | | | | | |
| partial cds; and nonstructural protein 1 (NS1) | | | | | | |
| gene, complete cds | | | | | | |
| Influenza B virus (B/Alaska/02/2019) segment | 37.4 | 37.4 | 100% | 0.02 | 95 | MN085436.1 |
| 8 nuclear export protein (NEP) and | | | | | | |
| nonstructural protein 1 (NS1) genes, complete | | | | | | |
| cds | | | | | | |
| Influenza B virus (B/Idaho/04/2019) segment 8 | 37.4 | 37.4 | 100% | 0.02 | 95 | MK998928.1 |
| nuclear export protein (NEP) and nonstructural | | | | | | |
| protein 1 (NS1) genes, complete cds | | | | | | |
| Influenza B virus (B/Uganda/S28_NP-S_E0- | 37.4 | 37.4 | 100% | 0.02 | 95 | MH684324.1 |
| 59/2013) segment 8 nuclear export protein | | | | | | |
| (NEP) and nonstructural protein 1 (NS1) genes, | | | | | | |
| partial cds | | | | | | |
| Influenza B virus (B/Uganda/S16_NP-S_E0- | 37.4 | 37.4 | 100% | 0.02 | 95 | MH684323.1 |
| 66/2013) segment 8 nuclear export protein | | | | | | |
| (NEP) gene, partial cds; and nonstructural | | | | | | |
| protein 1 (NS1) gene, complete cds | | | | | | |
| Influenza B virus (B/Nicaragua/6545_10/2012) | 37.4 | 37.4 | 100% | 0.02 | 95 | MK961756.1 |
| segment 8 nuclear export protein (NEP) and | | | | | | |
| nonstructural protein 1 (NS1) genes, complete | | | | | | |
| cds | | | | | | |
| Influenza B virus (B/Nicaragua/5350_03/2012) | 37.4 | 37.4 | 100% | 0.02 | 95 | MK961724.1 |
| segment 8 nuclear export protein (NEP) and | | | | | | |
| nonstructural protein 1 (NS1) genes, complete | | | | | | |
| cds | | | | | | |
| Influenza B virus (B/Nicaragua/2972_02/2012) | 37.4 | 37.4 | 100% | 0.02 | 95 | MK961685.1 |
| segment 8 nuclear export protein (NEP) and | | | | | | |
| nonstructural protein 1 (NS1) genes, complete | | | | | | |
| cds | | | | | | |
| Influenza B virus (B/Nicaragua/4430_01/2012) | 37.4 | 37.4 | 100% | 0.02 | 95 | MK961581.1 |
| segment 8 nuclear export protein (NEP) and | | | | | | |
| nonstructural protein 1 (NS1) genes, complete | | | | | | |
| cds | | | | | | |
| Influenza B virus (B/Nicaragua/5342_02/2012) | 37.4 | 37.4 | 100% | 0.02 | 95 | MK961328.1 |
| segment 8 nuclear export protein (NEP) and | | | | | | |
| nonstructural protein 1 (NS1) genes, complete | | | | | | |
| cds | | | | | | |
| Influenza B virus (B/Washington/276/2018) | 37.4 | 37.4 | 100% | 0.02 | 95 | MK474515.1 |
| segment 8 nuclear export protein (NEP) and | | | | | | |
| nonstructural protein 1 (NS1) genes, complete | | | | | | |
| cds | | | | | | |
| Influenza B virus (B/Hong Kong/8038/2011) | 37.4 | 37.4 | 100% | 0.02 | 95 | MH636855.1 |
| segment 8 nuclear export protein (NEP) and | | | | | | |
| nonstructural protein 1 (NS1) genes, complete | | | | | | |
| cds | | | | | | |
| Influenza B virus (B/North Carolina/14/2018) | 37.4 | 37.4 | 100% | 0.02 | 95 | MH245602.1 |
| segment 8 nuclear export protein (NEP) and | | | | | | |
| nonstructural protein 1 (NS1) genes, complete | | | | | | |
| cds | | | | | | |
| Influenza B virus (B/Bethesda/NIH001/2007) | 37.4 | 37.4 | 100% | 0.02 | 95 | MG869088.1 |
| segment 8 nuclear export protein (NEP) and | | | | | | |
| nonstructural protein 1 (NS1) genes, complete | | | | | | |
| cds | | | | | | |
| Influenza B virus (B/Nicaragua/6187_03/2012) | 37.4 | 37.4 | 100% | 0.02 | 95 | CY264005.1 |
| nuclear export protein (NEP) and nonstructural | | | | | | |
| protein 1 (NS1) genes, complete cds | | | | | | |
| Influenza B virus (B/Maryland/28/2017) | 37.4 | 37.4 | 100% | 0.02 | 95 | CY247345.1 |
| nuclear export protein (NEP) and nonstructural | | | | | | |
| protein 1 (NS1) genes, complete cds | | | | | | |
| Influenza B virus (B/Minnesota/20/2017) | 37.4 | 37.4 | 100% | 0.02 | 95 | CY234140.1 |
| nuclear export protein (NEP) and nonstructural | | | | | | |
| protein 1 (NS1) genes, complete cds | | | | | | |
| Influenza B virus (B/West Virginia/12/2017) | 37.4 | 37.4 | 100% | 0.02 | 95 | CY233972.1 |
| nuclear export protein (NEP) and nonstructural | | | | | | |
| protein 1 (NS1) genes, complete cds | | | | | | |
| Influenza B virus (B/Massachusetts/13/2017) | 37.4 | 37.4 | 100% | 0.02 | 95 | CY233516.1 |
| nuclear export protein (NEP) and nonstructural | | | | | | |
| protein 1 (NS1) genes, complete cds | | | | | | |
| Influenza B virus (B/Michigan/32/2017) | 37.4 | 37.4 | 100% | 0.02 | 95 | CY233460.1 |
| nuclear export protein (NEP) and nonstructural | | | | | | |
| protein 1 (NS1) genes, complete cds | | | | | | |
| Influenza B virus (B/Alabama/06/2017) nuclear | 37.4 | 37.4 | 100% | 0.02 | 95 | CY231917.1 |
| export protein (NEP) and nonstructural protein | | | | | | |
| 1 (NS1) genes, complete cds | | | | | | |
| Influenza B virus (B/Michigan/22/2017) | 37.4 | 37.4 | 100% | 0.02 | 95 | CY229881.1 |
| nuclear export protein (NEP) and nonstructural | | | | | | |
| protein 1 (NS1) genes, complete cds | | | | | | |
| Influenza B virus (B/Connecticut/18/2017) | 37.4 | 37.4 | 100% | 0.02 | 95 | CY229489.1 |
| nuclear export protein (NEP) and nonstructural | | | | | | |
| protein 1 (NS1) genes, complete cds | | | | | | |
| Influenza B virus (B/Malaysia/U2462/2013) | 37.4 | 37.4 | 100% | 0.02 | 95 | KX270006.1 |
| segment 8 nuclear export protein (NEP) and | | | | | | |
| nonstructural protein 1 (NS1) genes, complete | | | | | | |
| cds | | | | | | |
| Influenza B virus (B/Malaysia/U1429/2012) | 37.4 | 37.4 | 100% | 0.02 | 95 | KX270004.1 |
| segment 8 nuclear export protein (NEP) and | | | | | | |
| nonstructural protein 1 (NS1) genes, complete | | | | | | |
| cds | | | | | | |
| Influenza B virus (B/Texas/09/2017) nuclear | 37.4 | 37.4 | 100% | 0.02 | 95 | CY218718.1 |
| export protein (NEP) and nonstructural protein | | | | | | |
| 1 (NS1) genes, complete cds | | | | | | |
| Influenza B virus (B/Massachusetts/03/2017) | 37.4 | 37.4 | 100% | 0.02 | 95 | CY217822.1 |
| nuclear export protein (NEP) and nonstructural | | | | | | |
| protein 1 (NS1) genes, complete cds | | | | | | |
| Influenza B virus (B/Michigan/60/2016) | 37.4 | 37.4 | 100% | 0.02 | 95 | CY212326.1 |
| nuclear export protein (NEP) and nonstructural | | | | | | |
| protein 1 (NS1) genes, complete cds | | | | | | |
| Influenza B virus (B/Arkansas/05/2016) | 37.4 | 37.4 | 100% | 0.02 | 95 | KX612232.1 |
| segment 8 nuclear export protein (NEP) and | | | | | | |
| nonstructural protein 1 (NS1) genes, complete | | | | | | |
| cds | | | | | | |
| Influenza B virus (B/Rochester/F134/2011) | 37.4 | 37.4 | 100% | 0.02 | 95 | KX351446.1 |
| segment 8 nonstructural protein 2 (NS2) and | | | | | | |
| nonstructural protein 1 (NS1) genes, complete | | | | | | |
| cds | | | | | | |
| Influenza B virus (B/Rochester/F157/2011) | 37.4 | 37.4 | 100% | 0.02 | 95 | KX351438.1 |
| segment 8 nonstructural protein 2 (NS2) and | | | | | | |
| nonstructural protein 1 (NS1) genes, complete | | | | | | |
| cds | | | | | | |
| Influenza B virus (B/Rochester/0027/2011) | 37.4 | 37.4 | 100% | 0.02 | 95 | KX351433.1 |
| segment 8 nonstructural protein 2 (NS2) and | | | | | | |
| nonstructural protein 1 (NS1) genes, complete | | | | | | |
| cds | | | | | | |
| Influenza B virus (B/Perth/35/2013) nuclear | 37.4 | 37.4 | 100% | 0.02 | 95 | CY198197.1 |
| export protein (NEP) and nonstructural protein | | | | | | |
| 1 (NS1) genes, complete cds | | | | | | |
| Influenza B virus (B/South Auckland/29/2012) | 37.4 | 37.4 | 100% | 0.02 | 95 | CY198037.1 |
| nuclear export protein (NEP) and nonstructural | | | | | | |
| protein 1 (NS1) genes, complete cds | | | | | | |
| Influenza B virus (B/Wellington/001/2014) | 37.4 | 37.4 | 100% | 0.02 | 95 | CY202581.1 |
| nuclear export protein (NEP) and nonstructural | | | | | | |
| protein 1 (NS1) genes, complete cds | | | | | | |
| Influenza B virus (B/Christchurch/536/2013) | 37.4 | 37.4 | 100% | 0.02 | 95 | CY202565.1 |
| nuclear export protein (NEP) and nonstructural | | | | | | |
| protein 1 (NS1) genes, complete cds | | | | | | |
| Influenza B virus (B/Victoria/506/2012) nuclear | 37.4 | 37.4 | 100% | 0.02 | 95 | CY202557.1 |
| export protein (NEP) and nonstructural protein | | | | | | |
| 1 (NS1) genes, complete cds | | | | | | |
| Influenza B virus (B/Brisbane/60/2014) nuclear | 37.4 | 37.4 | 100% | 0.02 | 95 | CY202437.1 |
| export protein (NEP) and nonstructural protein | | | | | | |
| 1 (NS1) genes, complete cds | | | | | | |
| Influenza B virus (B/Townsville/7/2014) | 37.4 | 37.4 | 100% | 0.02 | 95 | CY202421.1 |
| nuclear export protein (NEP) and nonstructural | | | | | | |
| protein 1 (NS1) genes, complete cds | | | | | | |
| Influenza B virus (B/Tasmania/7/2014) nuclear | 37.4 | 37.4 | 100% | 0.02 | 95 | CY202405.1 |
| export protein (NEP) and nonstructural protein | | | | | | |
| 1 (NS1) genes, complete cds | | | | | | |
| Influenza B virus (B/Tasmania/5/2014) nuclear | 37.4 | 37.4 | 100% | 0.02 | 95 | CY202365.1 |
| export protein (NEP) and nonstructural protein | | | | | | |
| 1 (NS1) genes, complete cds | | | | | | |
| Influenza B virus (B/Newcastle/24/2014) | 37.4 | 37.4 | 100% | 0.02 | 95 | CY202349.1 |
| nuclear export protein (NEP) and nonstructural | | | | | | |
| protein 1 (NS1) genes, complete cds | | | | | | |
| Influenza B virus (B/Wellington/21/2014) | 37.4 | 37.4 | 100% | 0.02 | 95 | CY202157.1 |
| nuclear export protein (NEP) and nonstructural | | | | | | |
| protein 1 (NS1) genes, complete cds | | | | | | |
| Influenza B virus (B/Brisbane/53/2014) nuclear | 37.4 | 37.4 | 100% | 0.02 | 95 | CY201965.1 |
| export protein (NEP) and nonstructural protein | | | | | | |
| 1 (NS1) genes, complete cds | | | | | | |
| Influenza B virus (B/Victoria/1/2014) nuclear | 37.4 | 37.4 | 100% | 0.02 | 95 | CY201765.1 |
| export protein (NEP) and nonstructural protein | | | | | | |
| 1 (NS1) genes, complete cds | | | | | | |
| Influenza B virus (B/South Australia/23/2014) | 37.4 | 37.4 | 100% | 0.02 | 95 | CY201453.1 |
| nuclear export protein (NEP) and nonstructural | | | | | | |
| protein 1 (NS1) genes, complete cds | | | | | | |
| JP 2020068763-A/19: METHOD FOR | 40.1 | 40.1 | 100% | 0.003 | 100 | MD231027.1 |
| INFLUENZA A VIRUS AND INFLUENZA B | | | | | | |
| VIRUS DETECTION | | | | | | |
| Sequence 82 from Patent WO2014195920 | 40.1 | 40.1 | 100% | 0.003 | 100 | JC997573.1 |
| Sequence 76 from Patent WO2014195920 | 40.1 | 40.1 | 100% | 0.003 | 100 | JC997567.1 |
| Sequence 18 from Patent WO2009080806 | 40.1 | 40.1 | 100% | 0.003 | 100 | HB465264.1 |
| Sequence 17 from Patent WO2009080806 | 40.1 | 40.1 | 100% | 0.003 | 100 | HB465263.1 |
| Sequence 16 from Patent WO2009080806 | 40.1 | 40.1 | 100% | 0.003 | 100 | HB465262.1 |
| Sequence 9 from Patent EP2072058 | 40.1 | 40.1 | 100% | 0.003 | 100 | HB403628.1 |
| Sequence 8 from Patent EP2072058 | 40.1 | 40.1 | 100% | 0.003 | 100 | HB403627.1 |
| Sequence 7 from Patent EP2072058 | 40.1 | 40.1 | 100% | 0.003 | 100 | HB403626.1 |
| Sequence 6 from Patent EP2072058 | 40.1 | 40.1 | 100% | 0.003 | 100 | HB403625.1 |
| Sequence 7628 from Patent WO2007130519 | 40.1 | 40.1 | 100% | 0.003 | 100 | GN357864.1 |
| Sequence 7627 from Patent WO2007130519 | 40.1 | 40.1 | 100% | 0.003 | 100 | GN357863.1 |
| Sequence 15 from Patent WO2009080806 | 40.1 | 40.1 | 100% | 0.003 | 100 | HB465261.1 |
| Sequence 32 from Patent WO2008143640 | 40.1 | 40.1 | 100% | 0.003 | 100 | GM968007.1 |
| Sequence 8 from Patent WO2004094466 | 40.1 | 40.1 | 100% | 0.003 | 100 | CQ903179.1 |
| MULTI PLASMID SYSTEM FOR THE | 40.1 | 40.1 | 100% | 0.003 | 100 | DD230359.1 |
| PRODUCTION OF INFLUENZA VIRUS | | | | | | |
| Influenza B virus (B/Lee/1940) segment 8, | 40.1 | 40.1 | 100% | 0.003 | 100 | NC 002211.1 |
| complete sequence | | | | | | |
| Influenza B virus (B/South Korea/9812/2019) | 40.1 | 40.1 | 100% | 0.003 | 100 | MT637904.1 |
| segment 8 nuclear export protein (NEP) and | | | | | | |
| nonstructural protein 1 (NS1) genes, complete | | | | | | |
| cds | | | | | | |
| Influenza B virus (B/Colorado/9810/2019) | 40.1 | 40.1 | 100% | 0.003 | 100 | MT637902.1 |
| segment 8 nuclear export protein (NEP) and | | | | | | |
| nonstructural protein 1 (NS1) genes, complete | | | | | | |
| cds | | | | | | |
| Influenza B virus (B/Minnesota/23/2020) | 40.1 | 40.1 | 100% | 0.003 | 100 | MT622887.1 |
| segment 8 nuclear export protein (NEP) and | | | | | | |
| nonstructural protein 1 (NS1) genes, complete | | | | | | |
| cds | | | | | | |
| Influenza B virus (B/Minnesota/22/2020) | 40.1 | 40.1 | 100% | 0.003 | 100 | MT622879.1 |
| segment 8 nuclear export protein (NEP) and | | | | | | |
| nonstructural protein 1 (NS1) genes, complete | | | | | | |
| cds | | | | | | |
| Influenza B virus (B/Hawaii/16/2020) segment | 40.1 | 40.1 | 100% | 0.003 | 100 | MT622871.1 |
| 8 nuclear export protein (NEP) and | | | | | | |
| nonstructural protein 1 (NS1) genes, complete | | | | | | |
| cds | | | | | | |
| Influenza B virus (B/Hawaii/15/2020) segment | 40.1 | 40.1 | 100% | 0.003 | 100 | MT622863.1 |
| 8 nuclear export protein (NEP) and | | | | | | |
| nonstructural protein 1 (NS1) genes, complete | | | | | | |
| cds | | | | | | |
| Influenza B virus (B/Washington/10134/2019) | 40.1 | 40.1 | 100% | 0.003 | 100 | MT600919.1 |
| segment 8 nuclear export protein (NEP) and | | | | | | |
| nonstructural protein 1 (NS1) genes, complete | | | | | | |
| cds | | | | | | |
| Influenza B virus (B/Washington/10133/2019) | 40.1 | 40.1 | 100% | 0.003 | 100 | MT600911.1 |
| segment 8 nuclear export protein (NEP) and | | | | | | |
| nonstructural protein 1 (NS1) genes, complete | | | | | | |
| cds | | | | | | |
| Influenza B virus (B/Virginia/10132/2019) | 40.1 | 40.1 | 100% | 0.003 | 100 | MT600903.1 |
| segment 8 nuclear export protein (NEP) and | | | | | | |
| nonstructural protein 1 (NS1) genes, complete | | | | | | |
| cds | | | | | | |
| Influenza B virus (B/Virginia/10131/2019) | 40.1 | 40.1 | 100% | 0.003 | 100 | MT600895.1 |
| segment 8 nuclear export protein (NEP) and | | | | | | |
| nonstructural protein 1 (NS1) genes, complete | | | | | | |
| cds | | | | | | |
| Influenza B virus (B/Virginia/10130/2019) | 40.1 | 40.1 | 100% | 0.003 | 100 | MT600887.1 |
| segment 8 nuclear export protein (NEP) and | | | | | | |
| nonstructural protein 1 (NS1) genes, complete | | | | | | |
| cds | | | | | | |
| Influenza B virus (B/Virginia/10128/2019) | 40.1 | 40.1 | 100% | 0.003 | 100 | MT600879.1 |
| segment 8 nuclear export protein (NEP) and | | | | | | |
| nonstructural protein 1 (NS1) genes, complete | | | | | | |
| cds | | | | | | |
| Influenza B virus (B/Texas/10125/2019) | 40.1 | 40.1 | 100% | 0.003 | 100 | MT600871.1 |
| segment 8 nuclear export protein (NEP) and | | | | | | |
| nonstructural protein 1 (NS1) genes, complete | | | | | | |
| cds | | | | | | |
| Influenza B virus (B/Texas/10124/2019) | 40.1 | 40.1 | 100% | 0.003 | 100 | MT600863.1 |
| segment 8 nuclear export protein (NEP) and | | | | | | |
| nonstructural protein 1 (NS1) genes, complete | | | | | | |
| cds | | | | | | |
| Influenza B virus (B/Texas/10123/2019) | 40.1 | 40.1 | 100% | 0.003 | 100 | MT600855.1 |
| segment 8 nuclear export protein (NEP) and | | | | | | |
| nonstructural protein 1 (NS1) genes, complete | | | | | | |
| cds | | | | | | |
| Influenza B virus (B/Texas/10122/2019) | 40.1 | 40.1 | 100% | 0.003 | 100 | MT600847.1 |
| segment 8 nuclear export protein (NEP) and | | | | | | |
| nonstructural protein 1 (NS1) genes, complete | | | | | | |
| cds | | | | | | |
| Influenza B virus (B/Texas/10121/2019) | 40.1 | 40.1 | 100% | 0.003 | 100 | MT600839.1 |
| segment 8 nuclear export protein (NEP) and | | | | | | |
| nonstructural protein 1 (NS1) genes, complete | | | | | | |
| cds | | | | | | |
| Influenza B virus (B/Texas/10118/2019) | 40.1 | 40.1 | 100% | 0.003 | 100 | MT600831.1 |
| segment 8 nuclear export protein (NEP) and | | | | | | |
| nonstructural protein 1 (NS1) genes, complete | | | | | | |
| cds | | | | | | |
| Influenza B virus (B/Texas/10117/2019) | 40.1 | 40.1 | 100% | 0.003 | 100 | MT600823.1 |
| segment 8 nuclear export protein (NEP) and | | | | | | |
| nonstructural protein 1 (NS1) genes, complete | | | | | | |
| cds | | | | | | |
| Influenza B virus (B/Texas/10116/2019) | 40.1 | 40.1 | 100% | 0.003 | 100 | MT600815.1 |
| segment 8 nuclear export protein (NEP) and | | | | | | |
| nonstructural protein 1 (NS1) genes, complete | | | | | | |
| cds | | | | | | |
| Influenza B virus (B/Texas/10115/2019) | 40.1 | 40.1 | 100% | 0.003 | 100 | MT600807.1 |
| segment 8 nuclear export protein (NEP) and | | | | | | |
| nonstructural protein 1 (NS1) genes, complete | | | | | | |
| cds | | | | | | |
| Influenza B virus (B/Texas/10114/2019) | 40.1 | 40.1 | 100% | 0.003 | 100 | MT600799.1 |
| segment 8 nuclear export protein (NEP) and | | | | | | |
| nonstructural protein 1 (NS1) genes, complete | | | | | | |
| cds | | | | | | |
| Influenza B virus (B/Texas/10113/2019) | 40.1 | 40.1 | 100% | 0.003 | 100 | MT600791.1 |
| segment 8 nuclear export protein (NEP) and | | | | | | |
| nonstructural protein 1 (NS1) genes, complete | | | | | | |
| cds | | | | | | |
| Influenza B virus (B/Texas/10112/2019) | 40.1 | 40.1 | 100% | 0.003 | 100 | MT600783.1 |
| segment 8 nuclear export protein (NEP) and | | | | | | |
| nonstructural protein 1 (NS1) genes, complete | | | | | | |
| cds | | | | | | |
| Influenza B virus (B/Texas/10111/2019) | 40.1 | 40.1 | 100% | 0.003 | 100 | MT600775.1 |
| segment 8 nuclear export protein (NEP) and | | | | | | |
| nonstructural protein 1 (NS1) genes, complete | | | | | | |
| cds | | | | | | |
| Influenza B virus (B/Texas/10110/2019) | 40.1 | 40.1 | 100% | 0.003 | 100 | MT600767.1 |
| segment 8 nuclear export protein (NEP) and | | | | | | |
| nonstructural protein 1 (NS1) genes, complete | | | | | | |
| cds | | | | | | |
| Influenza B virus (B/Texas/10108/2019) | 40.1 | 40.1 | 100% | 0.003 | 100 | MT600759.1 |
| segment 8 nuclear export protein (NEP) and | | | | | | |
| nonstructural protein 1 (NS1) genes, complete | | | | | | |
| cds | | | | | | |
| Influenza B virus (B/Texas/10103/2019) | 40.1 | 40.1 | 100% | 0.003 | 100 | MT600751.1 |
| segment 8 nuclear export protein (NEP) and | | | | | | |
| nonstructural protein 1 (NS1) genes, complete | | | | | | |
| cds | | | | | | |
| Influenza B virus (B/Texas/10102/2019) | 40.1 | 40.1 | 100% | 0.003 | 100 | MT600743.1 |
| segment 8 nuclear export protein (NEP) and | | | | | | |
| nonstructural protein 1 (NS1) genes, complete | | | | | | |
| cds | | | | | | |
| Influenza B virus (B/Texas/10101/2019) | 40.1 | 40.1 | 100% | 0.003 | 100 | MT600735.1 |
| segment 8 nuclear export protein (NEP) and | | | | | | |
| nonstructural protein 1 (NS1) genes, complete | | | | | | |
| cds | | | | | | |
| Influenza B virus (B/Texas/10100/2019) | 40.1 | 40.1 | 100% | 0.003 | 100 | MT600727.1 |
| segment 8 nuclear export protein (NEP) and | | | | | | |
| nonstructural protein 1 (NS1) genes, complete | | | | | | |
| cds | | | | | | |
| Influenza B virus (B/Texas/10098/2019) | 40.1 | 40.1 | 100% | 0.003 | 100 | MT600719.1 |
| segment 8 nuclear export protein (NEP) and | | | | | | |
| nonstructural protein 1 (NS1) genes, complete | | | | | | |
| cds | | | | | | |
| Influenza B virus (B/South | 40.1 | 40.1 | 100% | 0.003 | 100 | MT600711.1 |
| Carolina/10097/2019) segment 8 nuclear export | | | | | | |
| protein (NEP) and nonstructural protein 1 | | | | | | |
| (NS1) genes, complete cds | | | | | | |
| Influenza B virus (B/Nevada/10096/2019) | 40.1 | 40.1 | 100% | 0.003 | 100 | MT600703.1 |
| segment 8 nuclear export protein (NEP) and | | | | | | |
| nonstructural protein 1 (NS1) genes, complete | | | | | | |
| cds | | | | | | |
| Influenza B virus (B/Nevada/10095/2019) | 40.1 | 40.1 | 100% | 0.003 | 100 | MT600695.1 |
| segment 8 nuclear export protein (NEP) and | | | | | | |
| nonstructural protein 1 (NS1) genes, complete | | | | | | |
| cds | | | | | | |
| Influenza B virus (B/Nevada/10094/2019) | 40.1 | 40.1 | 100% | 0.003 | 100 | MT600687.1 |
| segment 8 nuclear export protein (NEP) and | | | | | | |
| nonstructural protein 1 (NS1) genes, complete | | | | | | |
| cds | | | | | | |
| Influenza B virus (B/Nevada/10093/2019) | 40.1 | 40.1 | 100% | 0.003 | 100 | MT600679.1 |
| segment 8 nuclear export protein (NEP) and | | | | | | |
| nonstructural protein 1 (NS1) genes, complete | | | | | | |
| cds | | | | | | |
| Influenza B virus (B/Nevada/10092/2019) | 40.1 | 40.1 | 100% | 0.003 | 100 | MT600671.1 |
| segment 8 nuclear export protein (NEP) and | | | | | | |
| nonstructural protein 1 (NS1) genes, complete | | | | | | |
| cds | | | | | | |
| Influenza B virus (B/Nevada/10091/2019) | 40.1 | 40.1 | 100% | 0.003 | 100 | MT600663.1 |
| segment 8 nuclear export protein (NEP) and | | | | | | |
| nonstructural protein 1 (NS1) genes, complete | | | | | | |
| cds | | | | | | |
| Influenza B virus (B/Nevada/10090/2019) | 40.1 | 40.1 | 100% | 0.003 | 100 | MT600655.1 |
| segment 8 nuclear export protein (NEP) and | | | | | | |
| nonstructural protein 1 (NS1) genes, complete | | | | | | |
| cds | | | | | | |
| Influenza B virus (B/Nevada/10088/2019) | 40.1 | 40.1 | 100% | 0.003 | 100 | MT600647.1 |
| segment 8 nuclear export protein (NEP) and | | | | | | |
| nonstructural protein 1 (NS1) genes, complete | | | | | | |
| cds | | | | | | |
| Influenza B virus (B/Nevada/10087/2019) | 40.1 | 40.1 | 100% | 0.003 | 100 | MT600639.1 |
| segment 8 nuclear export protein (NEP) and | | | | | | |
| nonstructural protein 1 (NS1) genes, complete | | | | | | |
| cds | | | | | | |
| Influenza B virus (B/Nevada/10085/2019) | 40.1 | 40.1 | 100% | 0.003 | 100 | MT600631.1 |
| segment 8 nuclear export protein (NEP) and | | | | | | |
| nonstructural protein 1 (NS1) genes, complete | | | | | | |
| cds | | | | | | |
| Influenza B virus (B/Nevada/10084/2019) | 40.1 | 40.1 | 100% | 0.003 | 100 | MT600623.1 |
| segment 8 nuclear export protein (NEP) and | | | | | | |
| nonstructural protein 1 (NS1) genes, complete | | | | | | |
| cds | | | | | | |
| Influenza B virus (B/Nevada/10083/2019) | 40.1 | 40.1 | 100% | 0.003 | 100 | MT600615.1 |
| segment 8 nuclear export protein (NEP) and | | | | | | |
| nonstructural protein 1 (NS1) genes, complete | | | | | | |
| cds | | | | | | |
| Influenza B virus (B/Nevada/10082/2019) | 40.1 | 40.1 | 100% | 0.003 | 100 | MT600607.1 |
| segment 8 nuclear export protein (NEP) and | | | | | | |
| nonstructural protein 1 (NS1) genes, complete | | | | | | |
| cds | | | | | | |
| Influenza B virus (B/Nevada/10081/2019) | 40.1 | 40.1 | 100% | 0.003 | 100 | MT600599.1 |
| segment 8 nuclear export protein (NEP) and | | | | | | |
| nonstructural protein 1 (NS1) genes, complete | | | | | | |
| cds | | | | | | |
| Influenza B virus (B/Nevada/10080/2019) | 40.1 | 40.1 | 100% | 0.003 | 100 | MT600591.1 |
| segment 8 nuclear export protein (NEP) and | | | | | | |
| nonstructural protein 1 (NS1) genes, complete | | | | | | |
| cds | | | | | | |
| Influenza B virus (B/Nevada/10077/2019) | 40.1 | 40.1 | 100% | 0.003 | 100 | MT600583.1 |
| segment 8 nuclear export protein (NEP) and | | | | | | |
| nonstructural protein 1 (NS1) genes, complete | | | | | | |
| cds | | | | | | |
| Influenza B virus (B/Nevada/10076/2019) | 40.1 | 40.1 | 100% | 0.003 | 100 | MT600575.1 |
| segment 8 nuclear export protein (NEP) and | | | | | | |
| nonstructural protein 1 (NS1) genes, complete | | | | | | |
| cds | | | | | | |
| Influenza B virus (B/Nevada/10075/2019) | 40.1 | 40.1 | 100% | 0.003 | 100 | MT600567.1 |
| segment 8 nuclear export protein (NEP) and | | | | | | |
| nonstructural protein 1 (NS1) genes, complete | | | | | | |
| cds | | | | | | |
| Influenza B virus (B/Nevada/10074/2019) | 40.1 | 40.1 | 100% | 0.003 | 100 | MT600559.1 |
| segment 8 nuclear export protein (NEP) and | | | | | | |
| nonstructural protein 1 (NS1) genes, complete | | | | | | |
| cds | | | | | | |
| Influenza B virus (B/Nevada/10073/2019) | 40.1 | 40.1 | 100% | 0.003 | 100 | MT600551.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10070/2019) | 40.1 | 40.1 | 100% | 0.003 | 100 | MT600543.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10069/2019) | 40.1 | 40.1 | 100% | 0.003 | 100 | MT600535.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10067/2019) | 40.1 | 40.1 | 100% | 0.003 | 100 | MT600527.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10066/2019) | 40.1 | 40.1 | 100% | 0.003 | 100 | MT600519.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10065/2019) | 40.1 | 40.1 | 100% | 0.003 | 100 | MT600511.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10064/2019) | 40.1 | 40.1 | 100% | 0.003 | 100 | MT600503.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10062/2019) | 40.1 | 40.1 | 100% | 0.003 | 100 | MT600495.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10061/2019) | 40.1 | 40.1 | 100% | 0.003 | 100 | MT600487.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10060/2019) | 40.1 | 40.1 | 100% | 0.003 | 100 | MT600479.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10058/2019) | 40.1 | 40.1 | 100% | 0.003 | 100 | MT600471.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10057/2019) | 40.1 | 40.1 | 100% | 0.003 | 100 | MT600463.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10055/2019) | 40.1 | 40.1 | 100% | 0.003 | 100 | MT600455.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10054/2019) | 40.1 | 40.1 | 100% | 0.003 | 100 | MT600447.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10052/2019) | 40.1 | 40.1 | 100% | 0.003 | 100 | MT600439.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10051/2019) | 40.1 | 40.1 | 100% | 0.003 | 100 | MT600431.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10050/2019) | 40.1 | 40.1 | 100% | 0.003 | 100 | MT600423.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10049/2019) | 40.1 | 40.1 | 100% | 0.003 | 100 | MT600415.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10047/2019) | 40.1 | 40.1 | 100% | 0.003 | 100 | MT600407.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10046/2019) | 40.1 | 40.1 | 100% | 0.003 | 100 | MT600399.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10045/2019) | 40.1 | 40.1 | 100% | 0.003 | 100 | MT600391.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10044/2019) | 40.1 | 40.1 | 100% | 0.003 | 100 | MT600383.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10043/2019) | 40.1 | 40.1 | 100% | 0.003 | 100 | MT600375.1 |
| segment 8 nuclear export protein (NEP) and | | | | | | |
| nonstructural protein 1 (NS1) genes, complete | | | | | | |
| cds | | | | | | |
| Influenza B virus (B/Mississippi/10042/2019) | 40.1 | 40.1 | 100% | 0.003 | 100 | MT600367.1 |
| segment 8 nuclear export protein (NEP) and | | | | | | |
| nonstructural protein 1 (NS1) genes, complete | | | | | | |
| cds | | | | | | |
| Influenza B virus (B/Kentucky/10040/2019) | 40.1 | 40.1 | 100% | 0.003 | 100 | MT600359.1 |
| segment 8 nuclear export protein (NEP) and | | | | | | |
| nonstructural protein 1 (NS1) genes, complete | | | | | | |
| cds | | | | | | |
| Influenza B virus (B/Florida/10038/2019) | 40.1 | 40.1 | 100% | 0.003 | 100 | MT600351.1 |
| segment 8 nuclear export protein (NEP) and | | | | | | |
| nonstructural protein 1 (NS1) genes, complete | | | | | | |
| cds | | | | | | |
| Influenza B virus (B/Florida/10037/2019) | 40.1 | 40.1 | 100% | 0.003 | 100 | MT600343.1 |
| segment 8 nuclear export protein (NEP) and | | | | | | |
| nonstructural protein 1 (NS1) genes, complete | | | | | | |
| cds | | | | | | |
| Influenza B virus (B/Colorado/10036/2019) | 40.1 | 40.1 | 100% | 0.003 | 100 | MT600335.1 |
| segment 8 nuclear export protein (NEP) and | | | | | | |
| nonstructural protein 1 (NS1) genes, complete | | | | | | |
| cds | | | | | | |
| Influenza B virus (B/Colorado/10035/2019) | 40.1 | 40.1 | 100% | 0.003 | 100 | MT600327.1 |
| segment 8 nuclear export protein (NEP) and | | | | | | |
| nonstructural protein 1 (NS1) genes, complete | | | | | | |
| cds | | | | | | |
| Influenza B virus (B/Arizona/10034/2019) | 40.1 | 40.1 | 100% | 0.003 | 100 | MT600319.1 |
| segment 8 nuclear export protein (NEP) and | | | | | | |
| nonstructural protein 1 (NS1) genes, complete | | | | | | |
| cds | | | | | | |
| Influenza B virus (B/Alaska/10032/2019) | 40.1 | 40.1 | 100% | 0.003 | 100 | MT600311.1 |
| segment 8 nuclear export protein (NEP) and | | | | | | |
| nonstructural protein 1 (NS1) genes, complete | | | | | | |
| cds | | | | | | |

In some embodiments, the composition described herein comprises probe 7. In additional embodiments, probe 7 may hybridize to a target nucleotide molecule that is a part of MERS-CoV, a novel coronavirus causing Middle East Respiratory Syndrome (MERS). In some embodiments, probe 7 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 7. In some embodiments, probe 7 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 7. Table 3 shows potential target nucleotide molecules in the Influenza C virus.

TABLE 3
Potential target nucleotide molecules in the Influenza C virus

| | Max | Total | Query | E | Per. | |
|---|---|---|---|---|---|---|
| Description | Score | Score | Cover | value | ident | Accession |
| Sequence 41 from Patent WO2008143640 | 34.2 | 34.2 | 100% | 0.093 | 100 | GM968016.1 |
| Influenza C virus (C/Ann Arbor/1/50) | 34.2 | 34.2 | 100% | 0.093 | 100 | NC_006311.1 |
| segment 5, complete sequence | | | | | | |
| Influenza C virus | 34.2 | 34.2 | 100% | 0.093 | 100 | MK480602.1 |
| (C/Thailand/CN38_NP_gene/2017) segment | | | | | | |
| 5 nucleoprotein (NP) gene, partial cds | | | | | | |
| Influenza C virus | 34.2 | 34.2 | 100% | 0.093 | 100 | MK480595.1 |
| (C/Thailand/B12983_NP_gene/2015) | | | | | | |
| segment 5 nucleoprotein (NP) gene, partial | | | | | | |
| cds | | | | | | |
| Influenza C virus | 34.2 | 34.2 | 100% | 0.093 | 100 | MK480588.1 |
| (C/Thailand/B12597_NP_gene/2015) | | | | | | |
| segment 5 nucleoprotein (NP) gene, partial | | | | | | |
| cds | | | | | | |
| Influenza C virus | 34.2 | 34.2 | 100% | 0.093 | 100 | MK480581.1 |
| (C/Thailand/c5395_NP_gene/2015) segment | | | | | | |
| 5 nucleoprotein (NP) gene, partial cds | | | | | | |
| Influenza C virus (C/Iwate/2/2016) NP gene | 34.2 | 34.2 | 100% | 0.093 | 100 | LC327663.1 |
| for nucleoprotein, complete cds | | | | | | |
| Influenza C virus | 34.2 | 34.2 | 100% | 0.093 | 100 | MH348117.1 |
| (C/Bovine/Montana/12/2016) segment 5 | | | | | | |
| nucleoprotein (NP) gene, complete cds | | | | | | |
| Influenza C virus (C/India/P119564/2011) | 34.2 | 34.2 | 100% | 0.093 | 100 | KT232086.1 |
| segment 5 nucleoprotein (NP) gene, complete | | | | | | |
| cds | | | | | | |
| Influenza C virus (C/Tokyo/4/2014) NP gene | 34.2 | 34.2 | 100% | 0.093 | 100 | LC123814.1 |
| for nucleoprotein, complete cds | | | | | | |
| Influenza C virus (C/Tokyo/1/2014) NP gene | 34.2 | 34.2 | 100% | 0.093 | 100 | LC123813.1 |
| for nucleoprotein, complete cds | | | | | | |
| Influenza C virus (C/Miyagi/6/2014) NP gene | 34.2 | 34.2 | 100% | 0.093 | 100 | LC123812.1 |
| for nucleoprotein, complete cds | | | | | | |
| Influenza C virus (C/Miyagi/2/2014) NP gene | 34.2 | 34.2 | 100% | 0.093 | 100 | LC123810.1 |
| for nucleoprotein, complete cds | | | | | | |
| Influenza C virus (C/Yamagata/33/2014) NP | 34.2 | 34.2 | 100% | 0.093 | 100 | LC123808.1 |
| gene for nucleoprotein, complete cds | | | | | | |
| Influenza C virus (C/Yamagata/32/2014) NP | 34.2 | 34.2 | 100% | 0.093 | 100 | LC123807.1 |
| gene for nucleoprotein, complete cds | | | | | | |
| Influenza C virus (C/Yamagata/30/2014) NP | 34.2 | 34.2 | 100% | 0.093 | 100 | LC123806.1 |
| gene for nucleoprotein, complete cds | | | | | | |
| Influenza C virus (C/Yamagata/16/2014) NP | 34.2 | 34.2 | 100% | 0.093 | 100 | LC123805.1 |
| gene for nucleoprotein, complete cds | | | | | | |
| Influenza C virus (C/Yamagata/13/2014) NP | 34.2 | 34.2 | 100% | 0.093 | 100 | LC123804.1 |
| gene for nucleoprotein, complete cds | | | | | | |
| Influenza C virus (C/Yamagata/6/2014) NP | 34.2 | 34.2 | 100% | 0.093 | 100 | LC123803.1 |
| gene for nucleoprotein, complete cds | | | | | | |
| Influenza C virus (C/Fukuoka/1/2012) NP | 34.2 | 34.2 | 100% | 0.093 | 100 | LC123802.1 |
| gene for nucleoprotein, complete cds | | | | | | |
| Influenza C virus (C/Miyagi/2/2012) NP gene | 34.2 | 34.2 | 100% | 0.093 | 100 | LC123801.1 |
| for nucleoprotein, complete cds | | | | | | |
| Influenza C virus (C/Yamagata/29/2012) NP | 34.2 | 34.2 | 100% | 0.093 | 100 | LC123800.1 |
| gene for nucleoprotein, complete cds | | | | | | |
| Influenza C virus (C/Yamagata/28/2012) NP | 34.2 | 34.2 | 100% | 0.093 | 100 | LC123799.1 |
| gene for nucleoprotein, complete cds | | | | | | |
| Influenza C virus (C/Yamagata/14/2012) NP | 34.2 | 34.2 | 100% | 0.093 | 100 | LC123798.1 |
| gene for nucleoprotein, complete cds | | | | | | |
| Influenza C virus (C/Yamagata/4/2012) NP | 34.2 | 34.2 | 100% | 0.093 | 100 | LC123796.1 |
| gene for nucleoprotein, complete cds | | | | | | |
| Influenza C virus (C/Yamagata/3/2012) NP | 34.2 | 34.2 | 100% | 0.093 | 100 | LC123795.1 |
| gene for nucleoprotein, complete cds | | | | | | |
| Influenza C virus (C/Yamagata/1/2012) NP | 34.2 | 34.2 | 100% | 0.093 | 100 | LC123794.1 |
| gene for nucleoprotein, complete cds | | | | | | |
| Influenza C virus (C/Tokyo/3/2010) NP gene | 34.2 | 34.2 | 100% | 0.093 | 100 | LC123793.1 |
| for nucleoprotein, complete cds | | | | | | |
| Influenza C virus (C/Miyagi/2/2008) NP gene | 34.2 | 34.2 | 100% | 0.093 | 100 | LC123791.1 |
| for nucleoprotein, complete cds | | | | | | |
| Influenza C virus (C/Yamagata/18/2008) NP | 34.2 | 34.2 | 100% | 0.093 | 100 | LC123790.1 |
| gene for nucleoprotein, complete cds | | | | | | |
| Influenza C virus (C/Yamagata/16/2008) NP | 34.2 | 34.2 | 100% | 0.093 | 100 | LC123789.1 |
| gene for nucleoprotein, complete cds | | | | | | |
| Influenza C virus (C/Yamagata/11/2008) NP | 34.2 | 34.2 | 100% | 0.093 | 100 | LC123788.1 |
| gene for nucleoprotein, complete cds | | | | | | |
| Influenza C virus (C/Yamagata/3/2008) NP | 34.2 | 34.2 | 100% | 0.093 | 100 | LC123787.1 |
| gene for nucleoprotein, complete cds | | | | | | |
| Influenza C virus (C/Miyagi/4/2006) NP gene | 34.2 | 34.2 | 100% | 0.093 | 100 | LC123786.1 |
| for nucleoprotein, complete cds | | | | | | |
| Influenza C virus (C/Yamagata/9/2006) NP | 34.2 | 34.2 | 100% | 0.093 | 100 | LC123782.1 |
| gene for nucleoprotein, complete cds | | | | | | |
| Influenza C virus (C/Fukuoka/2/2006) NP | 34.2 | 34.2 | 100% | 0.093 | 100 | LC123780.1 |
| gene for nucleoprotein, complete cds | | | | | | |
| Influenza C virus (C/Fukuoka/1/2005) NP | 34.2 | 34.2 | 100% | 0.093 | 100 | LC123779.1 |
| gene for nucleoprotein, complete cds | | | | | | |
| Influenza C virus (C/Yamagata/2/2005) NP | 34.2 | 34.2 | 100% | 0.093 | 100 | LC123776.1 |
| gene for nucleoprotein, complete cds | | | | | | |
| Influenza C virus (C/Miyagi/47/2004) NP | 34.2 | 34.2 | 100% | 0.093 | 100 | LC123774.1 |
| gene for nucleoprotein, complete cds | | | | | | |
| Influenza C virus (C/Miyagi/42/2004) NP | 34.2 | 34.2 | 100% | 0.093 | 100 | LC123773.1 |
| gene for nucleoprotein, complete cds | | | | | | |
| Influenza C virus (C/Miyagi/12/2004) NP | 34.2 | 34.2 | 100% | 0.093 | 100 | LC123771.1 |
| gene for nucleoprotein, complete cds | | | | | | |
| Influenza C virus (C/Yamagata/26/2004) NP | 34.2 | 34.2 | 100% | 0.093 | 100 | LC123769.1 |
| gene for nucleoprotein, complete cds | | | | | | |
| Influenza C virus (C/Yamagata/14/2004) NP | 34.2 | 34.2 | 100% | 0.093 | 100 | LC123767.1 |
| gene for nucleoprotein, complete cds | | | | | | |
| Influenza C virus (C/Yamagata/3/2004) NP | 34.2 | 34.2 | 100% | 0.093 | 100 | LC123766.1 |
| gene for nucleoprotein, complete cds | | | | | | |
| Influenza C virus (C/Miyagi/31/2002) NP | 34.2 | 34.2 | 100% | 0.093 | 100 | LC123765.1 |
| gene for nucleoprotein, complete cds | | | | | | |
| Influenza C virus (C/Miyagi/28/2002) NP | 34.2 | 34.2 | 100% | 0.093 | 100 | LC123764.1 |
| gene for nucleoprotein, complete cds | | | | | | |
| Influenza C virus (C/Miyagi/20/2002) NP | 34.2 | 34.2 | 100% | 0.093 | 100 | LC123763.1 |
| gene for nucleoprotein, complete cds | | | | | | |
| Influenza C virus (C/Miyagi/11/2002) NP | 34.2 | 34.2 | 100% | 0.093 | 100 | LC123762.1 |
| gene for nucleoprotein, complete cds | | | | | | |
| Influenza C virus (C/Yamagata/7/2002) NP | 34.2 | 34.2 | 100% | 0.093 | 100 | LC123761.1 |
| gene for nucleoprotein, complete cds | | | | | | |
| Influenza C virus (C/Yamagata/5/2002) NP | 34.2 | 34.2 | 100% | 0.093 | 100 | LC123760.1 |
| gene for nucleoprotein, complete cds | | | | | | |
| Influenza C virus (C/Yamagata/6/2000) NP | 34.2 | 34.2 | 100% | 0.093 | 100 | LC123759.1 |
| gene for nucleoprotein, complete cds | | | | | | |
| Influenza C virus (C/Aichi/1/99) NP gene for | 34.2 | 34.2 | 100% | 0.093 | 100 | LC123758.1 |
| nucleoprotein, complete cds | | | | | | |
| Influenza C virus (C/Yamagata/13/98) NP | 34.2 | 34.2 | 100% | 0.093 | 100 | LC123757.1 |
| gene for nucleoprotein, complete cds | | | | | | |
| Influenza C virus (C/Yamagata/8/96) NP | 34.2 | 34.2 | 100% | 0.093 | 100 | LC123756.1 |
| gene for nucleoprotein, complete cds | | | | | | |
| Influenza C virus (C/Miyagi/9/96) NP gene | 34.2 | 34.2 | 100% | 0.093 | 100 | LC123755.1 |
| for nucleoprotein, complete cds | | | | | | |
| Influenza C virus (C/Miyagi/8/96) NP gene | 34.2 | 34.2 | 100% | 0.093 | 100 | LC123754.1 |
| for nucleoprotein, complete cds | | | | | | |
| Influenza C virus (C/Miyagi/7/93) NP gene | 34.2 | 34.2 | 100% | 0.093 | 100 | LC123753.1 |
| for nucleoprotein, complete cds | | | | | | |
| Influenza C virus (C/Miyagi/1/93) NP gene | 34.2 | 34.2 | 100% | 0.093 | 100 | LC123751.1 |
| for nucleoprotein, complete cds | | | | | | |
| Influenza C virus (C/Yamagata/1/93) NP | 34.2 | 34.2 | 100% | 0.093 | 100 | LC123750.1 |
| gene for nucleoprotein, complete cds | | | | | | |
| Influenza C virus (C/Yamagata/4/92) NP | 34.2 | 34.2 | 100% | 0.093 | 100 | LC123748.1 |
| gene for nucleoprotein, complete cds | | | | | | |
| Influenza C virus (C/Miyagi/5/91) NP gene | 34.2 | 34.2 | 100% | 0.093 | 100 | LC123747.1 |
| for nucleoprotein, complete cds | | | | | | |
| Influenza C virus (C/Yamagata/10/89) NP | 34.2 | 34.2 | 100% | 0.093 | 100 | LC123746.1 |
| gene for nucleoprotein, complete cds | | | | | | |
| Influenza C virus (C/Yamagata/9/88) NP | 34.2 | 34.2 | 100% | 0.093 | 100 | LC123745.1 |
| gene for nucleoprotein, complete cds | | | | | | |
| Influenza C virus (C/Yamagata/7/88) NP | 34.2 | 34.2 | 100% | 0.093 | 100 | LC123744.1 |
| gene for nucleoprotein, complete cds | | | | | | |
| Influenza C virus (C/England/892/1983) NP | 34.2 | 34.2 | 100% | 0.093 | 100 | LC123743.1 |
| gene for nucleoprotein, complete cds | | | | | | |
| Influenza C virus (C/Nara/82) NP gene for | 34.2 | 34.2 | 100% | 0.093 | 100 | LC123742.1 |
| nucleoprotein, complete cds | | | | | | |
| Influenza C virus (C/Sao Paulo/378/82) NP | 34.2 | 34.2 | 100% | 0.093 | 100 | LC123741.1 |
| gene for nucleoprotein, complete cds | | | | | | |
| Influenza C virus (C/pig/Beijing/115/81) NP | 34.2 | 34.2 | 100% | 0.093 | 100 | LC123740.1 |
| gene for nucleoprotein, complete cds | | | | | | |
| Influenza C virus (C/Yamagata/7/81) NP | 34.2 | 34.2 | 100% | 0.093 | 100 | LC123737.1 |
| gene for nucleoprotein, complete cds | | | | | | |
| Influenza C virus (C/Aichi/1/81) NP gene for | 34.2 | 34.2 | 100% | 0.093 | 100 | LC123736.1 |
| nucleoprotein, complete cds | | | | | | |
| Influenza C virus (C/Mississippi/80) NP gene | 34.2 | 34.2 | 100% | 0.093 | 100 | LC123735.1 |
| for nucleoprotein, complete cds | | | | | | |
| Influenza C virus (C/Shizuoka/79) NP gene | 34.2 | 34.2 | 100% | 0.093 | 100 | LC123734.1 |
| for nucleoprotein, complete cds | | | | | | |
| Influenza C virus (C/Kyoto/1/79) NP gene for | 34.2 | 34.2 | 100% | 0.093 | 100 | LC123733.1 |
| nucleoprotein, complete cds | | | | | | |
| Influenza C virus (C/Kansas/1/79) NP gene | 34.2 | 34.2 | 100% | 0.093 | 100 | LC123732.1 |
| for nucleoprotein, complete cds | | | | | | |
| Influenza C virus (C/California/78) NP gene | 34.2 | 34.2 | 100% | 0.093 | 100 | LC123731.1 |
| for nucleoprotein, complete cds | | | | | | |
| Influenza C virus (C/Miyagi/77) NP gene for | 34.2 | 34.2 | 100% | 0.093 | 100 | LC123730.1 |
| nucleoprotein, complete cds | | | | | | |
| Influenza C virus (C/Kanagawa/1/76) NP | 34.2 | 34.2 | 100% | 0.093 | 100 | LC123729.1 |
| gene for nucleoprotein, complete cds | | | | | | |
| Influenza C virus (C/Aomori/74) NP gene for | 34.2 | 34.2 | 100% | 0.093 | 100 | LC123728.1 |
| nucleoprotein, complete cds | | | | | | |
| Influenza C virus (C/Sapporo/71) NP gene | 34.2 | 34.2 | 100% | 0.093 | 100 | LC123727.1 |
| for nucleoprotein, complete cds | | | | | | |
| Influenza C virus (C/Georgia/1/69) NP gene | 34.2 | 34.2 | 100% | 0.093 | 100 | LC123726.1 |
| for nucleoprotein, complete cds | | | | | | |
| Influenza C virus (C/Paris/1/67) NP gene for | 34.2 | 34.2 | 100% | 0.093 | 100 | LC123725.1 |
| nucleoprotein, complete cds | | | | | | |
| Influenza C virus (C/Yamagata/64) NP gene | 34.2 | 34.2 | 100% | 0.093 | 100 | LC123724.1 |
| for nucleoprotein, complete cds | | | | | | |
| Influenza C virus (C/Great Lakes/1167/1954) | 34.2 | 34.2 | 100% | 0.093 | 100 | LC123723.1 |
| NP gene for nucleoprotein, complete cds | | | | | | |
| Influenza C virus (C/Taylor/1233/1947) NP | 34.2 | 34.2 | 100% | 0.093 | 100 | LC123722.1 |
| gene for nucleoprotein, complete cds | | | | | | |
| Influenza C virus (C/India/P121719/2012) | 34.2 | 34.2 | 100% | 0.093 | 100 | KM507549.1 |
| segment 5 nucleoprotein (NP) gene, partial | | | | | | |
| cds | | | | | | |
| Influenza C virus (C/India/P135047/2013) | 34.2 | 34.2 | 100% | 0.093 | 100 | KM507556.1 |
| segment 5 nucleoprotein (NP) gene, complete | | | | | | |
| cds | | | | | | |
| Influenza C virus (C/Scotland/6855/2007) | 34.2 | 34.2 | 100% | 0.093 | 100 | KT835360.1 |
| segment 5 nucleoprotein (NP) gene, partial | | | | | | |
| cds | | | | | | |
| Influenza C virus (C/Scotland/5334/2007) | 34.2 | 34.2 | 100% | 0.093 | 100 | KT835359.1 |
| segment 5 nucleoprotein (NP) gene, partial | | | | | | |
| cds | | | | | | |
| Influenza C virus (C/Scotland/7274/2007) | 34.2 | 34.2 | 100% | 0.093 | 100 | KT835358.1 |
| segment 5 nucleoprotein (NP) gene, partial | | | | | | |
| cds | | | | | | |
| Influenza C virus (C/Scotland/7482/2007) | 34.2 | 34.2 | 100% | 0.093 | 100 | KT835357.1 |
| segment 5 nucleoprotein (NP) gene, partial | | | | | | |
| cds | | | | | | |
| Influenza C virus (C/Scotland/7383/2007) | 34.2 | 34.2 | 100% | 0.093 | 100 | KT835356.1 |
| segment 5 nucleoprotein (NP) gene, partial | | | | | | |
| cds | | | | | | |
| Influenza C virus (C/Scotland/5500/2007) | 34.2 | 34.2 | 100% | 0.093 | 100 | KT835355.1 |
| segment 5 nucleoprotein (NP) gene, partial | | | | | | |
| cds | | | | | | |
| Influenza C virus (C/Victoria/2/2012) | 34.2 | 34.2 | 100% | 0.093 | 100 | KM504281.1 |
| segment 5 nucleoprotein gene, complete cds | | | | | | |
| Influenza C virus (C/Singapore/DSO- | 34.2 | 34.2 | 100% | 0.093 | 100 | JX963111.1 |
| 070170/2006) segment 5 nucleoprotein (NP) | | | | | | |
| gene, partial cds | | | | | | |
| Influenza C virus (C/Eastern | 34.2 | 34.2 | 100% | 0.093 | 100 | JX947862.1 |
| India/1202/2011) segment 5 nucleoprotein | | | | | | |
| (NP) gene, partial cds | | | | | | |
| Influenza C virus (C/Yamagata/2/2010) NP | 34.2 | 34.2 | 100% | 0.093 | 100 | AB684316.1 |
| gene for nucleoprotein, complete cds | | | | | | |
| Influenza C virus (C/Yamagata/1/2007) NP | 34.2 | 34.2 | 100% | 0.093 | 100 | AB684315.1 |
| gene for nucleoprotein, complete cds | | | | | | |
| Influenza C virus (C/Yamagata/15/2004) NP | 34.2 | 34.2 | 100% | 0.093 | 100 | AB684313.1 |
| gene for nucleoprotein, complete cds | | | | | | |
| Influenza C virus (C/Miyagi/4/2002) NP gene | 34.2 | 34.2 | 100% | 0.093 | 100 | AB684312.1 |
| for nucleoprotein, complete cds | | | | | | |
| Influenza C virus (C/Yamagata/3/2000) NP | 34.2 | 34.2 | 100% | 0.093 | 100 | AB684311.1 |
| gene for nucleoprotein, complete cds | | | | | | |
| Influenza C virus | 40.1 | 40.1 | 100% | 0.003 | 100 | MK480602.1 |
| (C/Thailand/CN38_NP_gene/2017) segment | | | | | | |
| 5 nucleoprotein (NP) gene, partial cds | | | | | | |
| Influenza C virus | 40.1 | 40.1 | 100% | 0.003 | 100 | MK480595.1 |
| (C/Thailand/B12983_NP_gene/2015) | | | | | | |
| segment 5 nucleoprotein (NP) gene, partial | | | | | | |
| cds | | | | | | |
| Influenza C virus | 40.1 | 40.1 | 100% | 0.003 | 100 | MK480588.1 |
| (C/Thailand/B12597_NP_gene/2015) | | | | | | |
| segment 5 nucleoprotein (NP) gene, partial | | | | | | |
| cds | | | | | | |
| Influenza C virus | 40.1 | 40. | 100% | 0.003 | 100 | MK480581.1 |
| (C/Thailand/c5395_NP_gene/2015) segment | | | | | | |
| 5 nucleoprotein (NP) gene, partial cds | | | | | | |
| Influenza C virus (C/Seoul/APD462/2015) | 40.1 | 40. | 100% | 0.003 | 100 | MK050100.1 |
| segment 5 nucleoprotein (NP) gene, complete | | | | | | |
| cds | | | | | | |
| Influenza C virus (C/Iwate/2/2016) NP gene | 40.1 | 40.1 | 100% | 0.003 | 100 | LC327663.1 |
| for nucleoprotein, complete cds | | | | | | |
| Influenza C virus (C/Iwate/1/2016) NP gene | 40.1 | 40.1 | 100% | 0.003 | 100 | LC327656.1 |
| for nucleoprotein, complete cds | | | | | | |
| Influenza C virus | 40.1 | 40.1 | 100% | 0.003 | 100 | MH348117.1 |
| (C/Bovine/Montana/12/2016) segment 5 | | | | | | |
| nucleoprotein (NP) gene, complete cds | | | | | | |
| Influenza C virus (C/India/P119564/2011) | 40.1 | 40.1 | 100% | 0.003 | 100 | KT232086.1 |
| segment 5 nucleoprotein (NP) gene, complete | | | | | | |
| cds | | | | | | |
| Influenza C virus (C/Tokyo/4/2014) NP gene | 40.1 | 40.1 | 100% | 0.003 | 100 | LC123814.1 |
| for nucleoprotein, complete cds | | | | | | |
| Influenza C virus (C/Tokyo/1/2014) NP gene | 40.1 | 40.1 | 100% | 0.003 | 100 | LC123813.1 |
| for nucleoprotein, complete cds | | | | | | |
| Influenza C virus (C/Miyagi/6/2014) NP gene | 40.1 | 40.1 | 100% | 0.003 | 100 | LC123812.1 |
| for nucleoprotein, complete cds | | | | | | |
| Influenza C virus (C/Miyagi/5/2014) NP gene | 40.1 | 40.1 | 100% | 0.003 | 100 | LC123811.1 |
| for nucleoprotein, complete cds | | | | | | |
| Influenza C virus (C/Miyagi/2/2014) NP gene | 40.1 | 40.1 | 100% | 0.003 | 100 | LC123810.1 |
| for nucleoprotein, complete cds | | | | | | |
| Influenza C virus (C/Yamagata/33/2014) NP | 40.1 | 40.1 | 100% | 0.003 | 100 | LC123808.1 |
| gene for nucleoprotein, complete cds | | | | | | |
| Influenza C virus (C/Yamagata/32/2014) NP | 40.1 | 40.1 | 100% | 0.003 | 100 | LC123807.1 |
| gene for nucleoprotein, complete cds | | | | | | |
| Influenza C virus (C/Yamagata/30/2014) NP | 40.1 | 40.1 | 100% | 0.003 | 100 | LC123806.1 |
| gene for nucleoprotein, complete cds | | | | | | |
| Influenza C virus (C/Yamagata/16/2014) NP | 40.1 | 40.1 | 100% | 0.003 | 100 | LC123805.1 |
| gene for nucleoprotein, complete cds | | | | | | |
| Influenza C virus (C/Yamagata/13/2014) NP | 40.1 | 40.1 | 100% | 0.003 | 100 | LC123804.1 |
| gene for nucleoprotein, complete cds | | | | | | |
| Influenza C virus (C/Yamagata/6/2014) NP | 40.1 | 40.1 | 100% | 0.003 | 100 | LC123803.1 |
| gene for nucleoprotein, complete cds | | | | | | |
| Influenza C virus (C/Fukuoka/1/2012) NP | 40.1 | 40.1 | 100% | 0.003 | 100 | LC123802.1 |
| gene for nucleoprotein, complete cds | | | | | | |
| Influenza C virus (C/Miyagi/2/2012) NP gene | 40.1 | 40.1 | 100% | 0.003 | 100 | LC123801.1 |
| for nucleoprotein, complete cds | | | | | | |
| Influenza C virus (C/Yamagata/29/2012) NP | 40.1 | 40.1 | 100% | 0.003 | 100 | LC123800.1 |
| gene for nucleoprotein, complete cds | | | | | | |
| Influenza C virus (C/Yamagata/28/2012) NP | 40.1 | 40.1 | 100% | 0.003 | 100 | LC123799.1 |
| gene for nucleoprotein, complete cds | | | | | | |
| Influenza C virus (C/Yamagata/14/2012) NP | 40.1 | 40.1 | 100% | 0.003 | 100 | LC123798.1 |
| gene for nucleoprotein, complete cds | | | | | | |
| Influenza C virus (C/Yamagata/4/2012) NP | 40.1 | 40.1 | 100% | 0.003 | 100 | LC123796.1 |
| gene for nucleoprotein, complete cds | | | | | | |
| Influenza C virus (C/Yamagata/3/2012) NP | 40.1 | 40.1 | 100% | 0.003 | 100 | LC123795.1 |
| gene for nucleoprotein, complete cds | | | | | | |
| Influenza C virus (C/Yamagata/1/2012) NP | 40.1 | 40.1 | 100% | 0.003 | 100 | LC123794.1 |
| gene for nucleoprotein, complete cds | | | | | | |
| Influenza C virus (C/Tokyo/3/2010) NP gene | 40.1 | 40.1 | 100% | 0.003 | 100 | LC123793.1 |
| for nucleoprotein, complete cds | | | | | | |
| Influenza C virus (C/Miyagi/2/2008) NP gene | 40.1 | 40.1 | 100% | 0.003 | 100 | LC123791.1 |
| for nucleoprotein, complete cds | | | | | | |
| Influenza C virus (C/Yamagata/18/2008) NP | 40.1 | 40.1 | 100% | 0.003 | 100 | LC123790.1 |
| gene for nucleoprotein, complete cds | | | | | | |
| Influenza C virus (C/Yamagata/16/2008) NP | 40.1 | 40.1 | 100% | 0.003 | 100 | LC123789.1 |
| gene for nucleoprotein, complete cds | | | | | | |
| Influenza C virus (C/Yamagata/11/2008) NP | 40.1 | 40.1 | 100% | 0.003 | 100 | LC123788.1 |
| gene for nucleoprotein, complete cds | | | | | | |
| Influenza C virus (C/Yamagata/3/2008) NP | 40.1 | 40.1 | 100% | 0.003 | 100 | LC123787.1 |
| gene for nucleoprotein, complete cds | | | | | | |
| Influenza C virus (C/Miyagi/4/2006) NP gene | 40.1 | 40.1 | 100% | 0.003 | 100 | LC123786.1 |
| for nucleoprotein, complete cds | | | | | | |
| Influenza C virus (C/Yamagata/9/2006) NP | 40.1 | 40.1 | 100% | 0.003 | 100 | LC123782.1 |
| gene for nucleoprotein, complete cds | | | | | | |
| Influenza C virus (C/Fukuoka/2/2006) NP | 40.1 | 40.1 | 100% | 0.003 | 100 | LC123780.1 |
| gene for nucleoprotein, complete cds | | | | | | |
| Influenza C virus (C/Fukuoka/1/2005) NP | 40.1 | 40.1 | 100% | 0.003 | 100 | LC123779.1 |
| gene for nucleoprotein, complete cds | | | | | | |
| Influenza C virus (C/Yamagata/2/2005) NP | 40.1 | 40.1 | 100% | 0.003 | 100 | LC123776.1 |
| gene for nucleoprotein, complete cds | | | | | | |
| Influenza C virus (C/Yamagata/1/2005) NP | 40.1 | 40.1 | 100% | 0.003 | 100 | LC123775.1 |
| gene for nucleoprotein, complete cds | | | | | | |
| Influenza C virus (C/Miyagi/47/2004) NP | 40.1 | 40.1 | 100% | 0.003 | 100 | LC123774.1 |
| gene for nucleoprotein, complete cds | | | | | | |
| Influenza C virus (C/Miyagi/42/2004) NP | 40.1 | 40.1 | 100% | 0.003 | 100 | LC123773.1 |
| gene for nucleoprotein, complete cds | | | | | | |
| Influenza C virus (C/Miyagi/12/2004) NP | 40.1 | 40.1 | 100% | 0.003 | 100 | LC123771.1 |
| gene for nucleoprotein, complete cds | | | | | | |
| Influenza C virus (C/Yamagata/27/2004) NP | 40.1 | 40.1 | 100% | 0.003 | 100 | LC123770.1 |
| gene for nucleoprotein, complete cds | | | | | | |
| Influenza C virus (C/Yamagata/18/2004) NP | 40.1 | 40.1 | 100% | 0.003 | 100 | LC123768.1 |
| gene for nucleoprotein, complete cds | | | | | | |
| Influenza C virus (C/Yamagata/3/2004) NP | 40.1 | 40.1 | 100% | 0.003 | 100 | LC123766.1 |
| gene for nucleoprotein, complete cds | | | | | | |
| Influenza C virus (C/Miyagi/31/2002) NP | 40.1 | 40.1 | 100% | 0.003 | 100 | LC123765.1 |
| gene for nucleoprotein, complete cds | | | | | | |
| Influenza C virus (C/Miyagi/28/2002) NP | 40.1 | 40.1 | 100% | 0.003 | 100 | LC123764.1 |
| gene for nucleoprotein, complete cds | | | | | | |
| Influenza C virus (C/Miyagi/20/2002) NP | 40.1 | 40.1 | 100% | 0.003 | 100 | LC123763.1 |
| gene for nucleoprotein, complete cds | | | | | | |
| Influenza C virus (C/Miyagi/11/2002) NP | 40.1 | 40.1 | 100% | 0.003 | 100 | LC123762.1 |
| gene for nucleoprotein, complete cds | | | | | | |
| Influenza C virus (C/Yamagata/7/2002) NP | 40.1 | 40.1 | 100% | 0.003 | 100 | LC123761.1 |
| gene for nucleoprotein, complete cds | | | | | | |
| Influenza C virus (C/Yamagata/5/2002) NP | 40.1 | 40.1 | 100% | 0.003 | 100 | LC123760.1 |
| gene for nucleoprotein, complete cds | | | | | | |
| Influenza C virus (C/Yamagata/6/2000) NP | 40.1 | 40.1 | 100% | 0.003 | 100 | LC123759.1 |
| gene for nucleoprotein, complete cds | | | | | | |
| Influenza C virus (C/Aichi/1/99) NP gene for | 40.1 | 40.1 | 100% | 0.003 | 100 | LC123758.1 |
| nucleoprotein, complete cds | | | | | | |
| Influenza C virus (C/Yamagata/13/98) NP | 40.1 | 40.1 | 100% | 0.003 | 100 | LC123757.1 |
| gene for nucleoprotein, complete cds | | | | | | |
| Influenza C virus (C/Yamagata/8/96) NP | 40.1 | 40.1 | 100% | 0.003 | 100 | LC123756.1 |
| gene for nucleoprotein, complete cds | | | | | | |
| Influenza C virus (C/Miyagi/9/96) NP gene | 40.1 | 40.1 | 100% | 0.003 | 100 | LC123755.1 |
| for nucleoprotein, complete cds | | | | | | |
| Influenza C virus (C/Miyagi/7/93) NP gene | 40.1 | 40.1 | 100% | 0.003 | 100 | LC123753.1 |
| for nucleoprotein, complete cds | | | | | | |
| Influenza C virus (C/Miyagi/3/93) NP gene | 40.1 | 40.1 | 100% | 0.003 | 100 | LC123752.1 |
| for nucleoprotein, complete cds | | | | | | |
| Influenza C virus (C/Miyagi/1/93) NP gene | 40.1 | 40.1 | 100% | 0.003 | 100 | LC123751.1 |
| for nucleoprotein, complete cds | | | | | | |
| Influenza C virus (C/Yamagata/1/93) NP | 40.1 | 40.1 | 100% | 0.003 | 100 | LC123750.1 |
| gene for nucleoprotein, complete cds | | | | | | |
| Influenza C virus (C/Yamagata/5/92) NP | 40.1 | 40.1 | 100% | 0.003 | 100 | LC123749.1 |
| gene for nucleoprotein, complete cds | | | | | | |
| Influenza C virus (C/Yamagata/4/92) NP | 40.1 | 40.1 | 100% | 0.003 | 100 | LC123748.1 |
| gene for nucleoprotein, complete cds | | | | | | |
| Influenza C virus (C/Miyagi/5/91) NP gene | 40.1 | 40.1 | 100% | 0.003 | 100 | LC123747.1 |
| for nucleoprotein, complete cds | | | | | | |
| Influenza C virus (C/Yamagata/10/89) NP | 40.1 | 40.1 | 100% | 0.003 | 100 | LC123746.1 |
| gene for nucleoprotein, complete cds | | | | | | |
| Influenza C virus (C/Yamagata/9/88) NP | 40.1 | 40.1 | 100% | 0.003 | 100 | LC123745.1 |
| gene for nucleoprotein, complete cds | | | | | | |
| Influenza C virus (C/Yamagata/7/88) NP | 40.1 | 40.1 | 100% | 0.003 | 100 | LC123744.1 |
| gene for nucleoprotein, complete cds | | | | | | |
| Influenza C virus (C/England/892/1983) NP | 40.1 | 40.1 | 100% | 0.003 | 100 | LC123743.1 |
| gene for nucleoprotein, complete cds | | | | | | |
| Influenza C virus (C/Nara/82) NP gene for | 40.1 | 40.1 | 100% | 0.003 | 100 | LC123742.1 |
| nucleoprotein, complete cds | | | | | | |
| Influenza C virus (C/Sao Paulo/378/82) NP | 40.1 | 40.1 | 100% | 0.003 | 100 | LC123741.1 |
| gene for nucleoprotein, complete cds | | | | | | |
| Influenza C virus (C/pig/Beijing/115/81) NP | 40.1 | 40.1 | 100% | 0.003 | 100 | LC123740.1 |
| gene for nucleoprotein, complete cds | | | | | | |
| Influenza C virus (C/Yamagata/7/81) NP | 40.1 | 40.1 | 100% | 0.003 | 100 | LC123737.1 |
| gene for nucleoprotein, complete cds | | | | | | |
| Influenza C virus (C/Aichi/1/81) NP gene for | 40.1 | 40.1 | 100% | 0.003 | 100 | LC123736.1 |
| nucleoprotein, complete cds | | | | | | |
| Influenza C virus (C/Mississippi/80) NP gene | 40.1 | 40.1 | 100% | 0.003 | 100 | LC123735.1 |
| for nucleoprotein, complete cds | | | | | | |
| Influenza C virus (C/Shizuoka/79) NP gene | 40.1 | 40.1 | 100% | 0.003 | 100 | LC123734.1 |
| for nucleoprotein, complete cds | | | | | | |
| Influenza C virus (C/Kyoto/1/79) NP gene for | 40.1 | 40.1 | 100% | 0.003 | 100 | LC123733.1 |
| nucleoprotein, complete cds | | | | | | |
| Influenza C virus (C/Kansas/1/79) NP gene | 40.1 | 40.1 | 100% | 0.003 | 100 | LC123732.1 |
| for nucleoprotein, complete cds | | | | | | |
| Influenza C virus (C/California/78) NP gene | 40.1 | 40.1 | 100% | 0.003 | 100 | LC123731.1 |
| for nucleoprotein, complete cds | | | | | | |
| Influenza C virus (C/Miyagi/77) NP gene for | 40.1 | 40.1 | 100% | 0.003 | 100 | LC123730.1 |
| nucleoprotein, complete cds | | | | | | |
| Influenza C virus (C/Kanagawa/1/76) NP | 40.1 | 40.1 | 100% | 0.003 | 100 | LC123729.1 |
| gene for nucleoprotein, complete cds | | | | | | |
| Influenza C virus (C/Aomori/74) NP gene for | 40.1 | 40.1 | 100% | 0.003 | 100 | LC123728.1 |
| nucleoprotein, complete cds | | | | | | |
| Influenza C virus (C/Sapporo/71) NP gene | 40.1 | 40.1 | 100% | 0.003 | 100 | LC123727.1 |
| for nucleoprotein, complete cds | | | | | | |
| Influenza C virus (C/Georgia/1/69) NP gene | 40.1 | 40.1 | 100% | 0.003 | 100 | LC123726.1 |
| for nucleoprotein, complete cds | | | | | | |
| Influenza C virus (C/Paris/1/67) NP gene for | 40.1 | 40.1 | 100% | 0.003 | 100 | LC123725.1 |
| nucleoprotein, complete cds | | | | | | |
| Influenza C virus (C/Yamagata/64) NP gene | 40.1 | 40.1 | 100% | 0.003 | 100 | LC123724.1 |
| for nucleoprotein, complete cds | | | | | | |
| Influenza C virus (C/Great Lakes/1167/1954) | 40.1 | 40.1 | 100% | 0.003 | 100 | LC123723.1 |
| NP gene for nucleoprotein, complete cds | | | | | | |
| Influenza C virus (C/Taylor/1233/1947) NP | 40.1 | 40.1 | 100% | 0.003 | 100 | LC123722.1 |
| gene for nucleoprotein, complete cds | | | | | | |
| Influenza C virus (C/India/P121719/2012) | 40.1 | 40.1 | 100% | 0.003 | 100 | KM507549.1 |
| segment 5 nucleoprotein (NP) gene, partial | | | | | | |
| cds | | | | | | |
| Influenza C virus (C/India/P135047/2013) | 40.1 | 40.1 | 100% | 0.003 | 100 | KM507556.1 |
| segment 5 nucleoprotein (NP) gene, complete | | | | | | |
| cds | | | | | | |
| Influenza C virus (C/Scotland/6855/2007) | 40.1 | 40.1 | 100% | 0.003 | 100 | KT835360.1 |
| segment 5 nucleoprotein (NP) gene, partial | | | | | | |
| cds | | | | | | |
| Influenza C virus (C/Scotland/5334/2007) | 40.1 | 40.1 | 100% | 0.003 | 100 | KT835359.1 |
| segment 5 nucleoprotein (NP) gene, partial | | | | | | |
| cds | | | | | | |
| Influenza C virus (C/Scotland/7274/2007) | 40.1 | 40.1 | 100% | 0.003 | 100 | KT835358.1 |
| segment 5 nucleoprotein (NP) gene, partial | | | | | | |
| cds | | | | | | |
| Influenza C virus (C/Scotland/7482/2007) | 40.1 | 40.1 | 100% | 0.003 | 100 | KT835357.1 |
| segment 5 nucleoprotein (NP) gene, partial | | | | | | |
| cds | | | | | | |
| Influenza C virus (C/Scotland/7383/2007) | 40.1 | 40.1 | 100% | 0.003 | 100 | KT835356.1 |
| segment 5 nucleoprotein (NP) gene, partial | | | | | | |
| cds | | | | | | |
| Influenza C virus (C/Scotland/5500/2007) | 40.1 | 40.1 | 100% | 0.003 | 100 | KT835355.1 |
| segment 5 nucleoprotein (NP) gene, partial | | | | | | |
| cds | | | | | | |
| Influenza C virus (C/Victoria/2/2012) | 40.1 | 40.1 | 100% | 0.003 | 100 | KM504281.1 |
| segment 5 nucleoprotein gene, complete cds | | | | | | |
| Influenza C virus (C/Singapore/DSO- | 40.1 | 40.1 | 100% | 0.003 | 100 | JX963113.1 |
| 070203/2006) segment 5 nucleoprotein (NP) | | | | | | |
| gene, partial cds | | | | | | |
| Influenza C virus (C/Singapore/DSO- | 40.1 | 40.1 | 100% | 0.003 | 100 | JX963111.1 |
| 070170/2006) segment 5 nucleoprotein (NP) | | | | | | |
| gene, partial cds | | | | | | |
| Influenza C virus (C/Singapore/DSO- | 40.1 | 40.1 | 100% | 0.003 | 100 | JX963110.1 |
| 050530/2006) segment 5 nucleoprotein (NP) | | | | | | |
| gene, partial cds | | | | | | |
| Influenza C virus (C/Eastern | 40.1 | 40.1 | 100% | 0.003 | 100 | JX947862.1 |
| India/1202/2011) segment 5 nucleoprotein | | | | | | |
| (NP) gene, partial cds | | | | | | |
| Sequence 41 from Patent WO2008143640 | 50.1 | 50.1 | 100% | 6.00E− | 100 | GM968016.1 |
| | | | | 06 | | |
| Influenza C virus (C/Ann Arbor/1/50) | 50.1 | 50.1 | 100% | 6.00E− | 100 | NC_006311.1 |
| segment 5, complete sequence | | | | 06 | | |
| Influenza C virus | 50.1 | 50.1 | 100% | 6.00E− | 100 | MK480602.1 |
| (C/Thailand/CN38_NP_gene/2017) segment | | | | 06 | | |
| 5 nucleoprotein (NP) gene, partial cds | | | | | | |
| Influenza C virus | 50.1 | 50.1 | 100% | 6.00E− | 100 | MK480595.1 |
| (C/Thailand/B12983_NP_gene/2015) | | | | 06 | | |
| segment 5 nucleoprotein (NP) gene, partial | | | | | | |
| cds | | | | | | |
| Influenza C virus | 50.1 | 50.1 | 100% | 6.00E− | 100 | MK480588.1 |
| (C/Thailand/B12597_NP_gene/2015) | | | | 06 | | |
| segment 5 nucleoprotein (NP) gene, partial | | | | | | |
| cds | | | | | | |
| Influenza C virus | 50.1 | 50.1 | 100% | 6.00E− | 100 | MK480581.1 |
| (C/Thailand/c5395_NP_gene/2015) segment | | | | 06 | | |
| 5 nucleoprotein (NP) gene, partial cds | | | | | | |
| Influenza C virus (C/Seoul/APD462/2015) | 50.1 | 50.1 | 100% | 6.00E− | 100 | MK050100.1 |
| segment 5 nucleoprotein (NP) gene, complete | | | | 06 | | |
| cds | | | | | | |
| Influenza C virus (C/Iwate/2/2016) NP gene | 50.1 | 50.1 | 100% | 6.00E− | 100 | LC327663.1 |
| for nucleoprotein, complete cds | | | | 06 | | |
| Influenza C virus (C/Iwate/1/2016) NP gene | 50.1 | 50.1 | 100% | 6.00E− | 100 | LC327656.1 |
| for nucleoprotein, complete cds | | | | 06 | | |
| Influenza C virus (C/India/P119564/2011) | 50.1 | 50.1 | 100% | 6.00E− | 100 | KT232086.1 |
| segment 5 nucleoprotein (NP) gene, complete | | | | 06 | | |
| cds | | | | | | |
| Influenza C virus (C/Tokyo/4/2014) NP gene | 50.1 | 50.1 | 100% | 6.00E− | 100 | LC123814.1 |
| for nucleoprotein, complete cds | | | | 06 | | |
| Influenza C virus (C/Tokyo/1/2014) NP gene | 50.1 | 50.1 | 100% | 6.00E− | 100 | LC123813.1 |
| for nucleoprotein, complete cds | | | | 06 | | |
| Influenza C virus (C/Miyagi/6/2014) NP gene | 50.1 | 50.1 | 100% | 6.00E− | 100 | LC123812.1 |
| for nucleoprotein, complete cds | | | | 06 | | |
| Influenza C virus (C/Miyagi/5/2014) NP gene | 50.1 | 50.1 | 100% | 6.00E− | 100 | LC123811.1 |
| for nucleoprotein, complete cds | | | | 06 | | |
| Influenza C virus (C/Miyagi/2/2014) NP gene | 50.1 | 50.1 | 100% | 6.00E− | 100 | LC123810.1 |
| for nucleoprotein, complete cds | | | | 06 | | |
| Influenza C virus (C/Yamagata/33/2014) NP | 50.1 | 50.1 | 100% | 6.00E− | 100 | LC123808.1 |
| gene for nucleoprotein, complete cds | | | | 06 | | |
| Influenza C virus (C/Yamagata/32/2014) NP | 50.1 | 50.1 | 100% | 6.00E− | 100 | LC123807.1 |
| gene for nucleoprotein, complete cds | | | | 06 | | |
| Influenza C virus (C/Yamagata/30/2014) NP | 50.1 | 50.1 | 100% | 6.00E− | 100 | LC123806.1 |
| gene for nucleoprotein, complete cds | | | | 06 | | |
| Influenza C virus (C/Yamagata/16/2014) NP | 50.1 | 50.1 | 100% | 6.00E− | 100 | LC123805.1 |
| gene for nucleoprotein, complete cds | | | | 06 | | |
| Influenza C virus (C/Yamagata/13/2014) NP | 50.1 | 50.1 | 100% | 6.00E− | 100 | LC123804.1 |
| gene for nucleoprotein, complete cds | | | | 06 | | |
| Influenza C virus (C/Yamagata/6/2014) NP | 50.1 | 50.1 | 100% | 6.00E− | 100 | LC123803.1 |
| gene for nucleoprotein, complete cds | | | | 06 | | |
| Influenza C virus (C/Fukuoka/1/2012) NP | 50.1 | 50.1 | 100% | 6.00E− | 100 | LC123802.1 |
| gene for nucleoprotein, complete cds | | | | 06 | | |
| Influenza C virus (C/Yamagata/28/2012) NP | 50.1 | 50.1 | 100% | 6.00E− | 100 | LC123799.1 |
| gene for nucleoprotein, complete cds | | | | 06 | | |
| Influenza C virus (C/Yamagata/4/2012) NP | 50.1 | 50.1 | 100% | 6.00E− | 100 | LC123796.1 |
| gene for nucleoprotein, complete cds | | | | 06 | | |
| Influenza C virus (C/Yamagata/1/2012) NP | 50.1 | 50.1 | 100% | 6.00E− | 100 | LC123794.1 |
| gene for nucleoprotein, complete cds | | | | 06 | | |
| Influenza C virus (C/Tokyo/3/2010) NP gene | 50.1 | 50.1 | 100% | 6.00E− | 100 | LC123793.1 |
| for nucleoprotein, complete cds | | | | 06 | | |
| Influenza C virus (C/Yamagata/18/2008) NP | 50.1 | 50.1 | 100% | 6.00E− | 100 | LC123790.1 |
| gene for nucleoprotein, complete cds | | | | 06 | | |
| Influenza C virus (C/Yamagata/16/2008) NP | 50.1 | 50.1 | 100% | 6.00E− | 100 | LC123789.1 |
| gene for nucleoprotein, complete cds | | | | 06 | | |
| Influenza C virus (C/Yamagata/11/2008) NP | 50.1 | 50.1 | 100% | 6.00E− | 100 | LC123788.1 |
| gene for nucleoprotein, complete cds | | | | 06 | | |
| Influenza C virus (C/Miyagi/4/2006) NP gene | 50.1 | 50.1 | 100% | 6.00E− | 100 | LC123786.1 |
| for nucleoprotein, complete cds | | | | 06 | | |
| Influenza C virus (C/Yamagata/9/2006) NP | 50.1 | 50.1 | 100% | 6.00E− | 100 | LC123782.1 |
| gene for nucleoprotein, complete cds | | | | 06 | | |
| Influenza C virus (C/Fukuoka/1/2005) NP | 50.1 | 50.1 | 100% | 6.00E− | 100 | LC123779.1 |
| gene for nucleoprotein, complete cds | | | | 06 | | |
| Influenza C virus (C/Yamagata/2/2005) NP | 50.1 | 50.1 | 100% | 6.00E− | 100 | LC123776.1 |
| gene for nucleoprotein, complete cds | | | | 06 | | |
| Influenza C virus (C/Yamagata/1/2005) NP | 50.1 | 50.1 | 100% | 6.00E− | 100 | LC123775.1 |
| gene for nucleoprotein, complete cds | | | | 06 | | |
| Influenza C virus (C/Miyagi/47/2004) NP | 50.1 | 50.1 | 100% | 6.00E− | 100 | LC123774.1 |
| gene for nucleoprotein, complete cds | | | | 06 | | |
| Influenza C virus (C/Miyagi/42/2004) NP | 50.1 | 50.1 | 100% | 6.00E− | 100 | LC123773.1 |
| gene for nucleoprotein, complete cds | | | | 06 | | |
| Influenza C virus (C/Miyagi/12/2004) NP | 50.1 | 50.1 | 100% | 6.00E− | 100 | LC123771.1 |
| gene for nucleoprotein, complete cds | | | | 06 | | |
| Influenza C virus (C/Yamagata/27/2004) NP | 50.1 | 50.1 | 100% | 6.00E− | 100 | LC123770.1 |
| gene for nucleoprotein, complete cds | | | | 06 | | |
| Influenza C virus (C/Yamagata/26/2004) NP | 50.1 | 50.1 | 100% | 6.00E− | 100 | LC123769.1 |
| gene for nucleoprotein, complete cds | | | | 06 | | |
| Influenza C virus (C/Yamagata/18/2004) NP | 50.1 | 50.1 | 100% | 6.00E− | 100 | LC123768.1 |
| gene for nucleoprotein, complete cds | | | | 06 | | |
| Influenza C virus (C/Yamagata/14/2004) NP | 50.1 | 50.1 | 100% | 6.00E− | 100 | LC123767.1 |
| gene for nucleoprotein, complete cds | | | | 06 | | |
| Influenza C virus (C/Yamagata/3/2004) NP | 50.1 | 50.1 | 100% | 6.00E− | 100 | LC123766.1 |
| gene for nucleoprotein, complete cds | | | | 06 | | |
| Influenza C virus (C/Miyagi/31/2002) NP | 50.1 | 50.1 | 100% | 6.00E− | 100 | LC123765.1 |
| gene for nucleoprotein, complete cds | | | | 06 | | |
| Influenza C virus (C/Miyagi/28/2002) NP | 50.1 | 50.1 | 100% | 6.00E− | 100 | LC123764.1 |
| gene for nucleoprotein, complete cds | | | | 06 | | |
| Influenza C virus (C/Miyagi/20/2002) NP | 50.1 | 50.1 | 100% | 6.00E− | 100 | LC123763.1 |
| gene for nucleoprotein, complete cds | | | | 06 | | |
| Influenza C virus (C/Miyagi/11/2002) NP | 50.1 | 50.1 | 100% | 6.00E− | 100 | LC123762.1 |
| gene for nucleoprotein, complete cds | | | | 06 | | |
| Influenza C virus (C/Yamagata/7/2002) NP | 50.1 | 50.1 | 100% | 6.00E− | 100 | LC123761.1 |
| gene for nucleoprotein, complete cds | | | | 06 | | |
| Influenza C virus (C/Yamagata/5/2002) NP | 50.1 | 50.1 | 100% | 6.00E− | 100 | LC123760.1 |
| gene for nucleoprotein, complete cds | | | | 06 | | |
| Influenza C virus (C/Yamagata/6/2000) NP | 50.1 | 50.1 | 100% | 6.00E− | 100 | LC123759.1 |
| gene for nucleoprotein, complete cds | | | | 06 | | |
| Influenza C virus (C/Aichi/1/99) NP gene for | 50.1 | 50.1 | 100% | 6.00E− | 100 | LC123758.1 |
| nucleoprotein, complete cds | | | | 06 | | |
| Influenza C virus (C/Yamagata/13/98) NP | 50.1 | 50.1 | 100% | 6.00E− | 100 | LC123757.1 |
| gene for nucleoprotein, complete cds | | | | 06 | | |
| Influenza C virus (C/Yamagata/8/96) NP | 50.1 | 50.1 | 100% | 6.00E− | 100 | LC123756.1 |
| gene for nucleoprotein, complete cds | | | | 06 | | |
| Influenza C virus (C/Miyagi/9/96) NP gene | 50.1 | 50.1 | 100% | 6.00E− | 100 | LC123755.1 |
| for nucleoprotein, complete cds | | | | 06 | | |
| Influenza C virus (C/Miyagi/8/96) NP gene | 50.1 | 50.1 | 100% | 6.00E− | 100 | LC123754.1 |
| for nucleoprotein, complete cds | | | | 06 | | |
| Influenza C virus (C/Miyagi/7/93) NP gene | 50.1 | 50.1 | 100% | 6.00E− | 100 | LC123753.1 |
| for nucleoprotein, complete cds | | | | 06 | | |
| Influenza C virus (C/Miyagi/3/93) NP gene | 50.1 | 50.1 | 100% | 6.00E− | 100 | LC123752.1 |
| for nucleoprotein, complete cds | | | | 06 | | |
| Influenza C virus (C/Miyagi/1/93) NP gene | 50.1 | 50.1 | 100% | 6.00E− | 100 | LC123751.1 |
| for nucleoprotein, complete cds | | | | 06 | | |
| Influenza C virus (C/Yamagata/5/92) NP | 50.1 | 50.1 | 100% | 6.00E− | 100 | LC123749.1 |
| gene for nucleoprotein, complete cds | | | | 06 | | |
| Influenza C virus (C/Yamagata/4/92) NP | 50.1 | 50.1 | 100% | 6.00E− | 100 | LC123748.1 |
| gene for nucleoprotein, complete cds | | | | 06 | | |
| Influenza C virus (C/Miyagi/5/91) NP gene | 50.1 | 50.1 | 100% | 6.00E− | 100 | LC123747.1 |
| for nucleoprotein, complete cds | | | | 06 | | |
| Influenza C virus (C/Yamagata/10/89) NP | 50.1 | 50.1 | 100% | 6.00E− | 100 | LC123746.1 |
| gene for nucleoprotein, complete cds | | | | 06 | | |
| Influenza C virus (C/Yamagata/9/88) NP | 50.1 | 50.1 | 100% | 6.00E− | 100 | LC123745.1 |
| gene for nucleoprotein, complete cds | | | | 06 | | |
| Influenza C virus (C/Yamagata/7/88) NP | 50.1 | 50.1 | 100% | 6.00E− | 100 | LC123744.1 |
| gene for nucleoprotein, complete cds | | | | 06 | | |
| Influenza C virus (C/England/892/1983) NP | 50.1 | 50.1 | 100% | 6.00E− | 100 | LC123743.1 |
| gene for nucleoprotein, complete cds | | | | 06 | | |
| Influenza C virus (C/Nara/82) NP gene for | 50.1 | 50.1 | 100% | 6.00E− | 100 | LC123742.1 |
| nucleoprotein, complete cds | | | | 06 | | |
| Influenza C virus (C/pig/Beijing/115/81) NP | 50.1 | 50.1 | 100% | 6.00E− | 100 | LC123740.1 |
| gene for nucleoprotein, complete cds | | | | 06 | | |
| Influenza C virus (C/Yamagata/7/81) NP | 50.1 | 50.1 | 100% | 6.00E− | 100 | LC123737.1 |
| gene for nucleoprotein, complete cds | | | | 06 | | |
| Influenza C virus (C/Aichi/1/81) NP gene for | 50.1 | 50.1 | 100% | 6.00E− | 100 | LC123736.1 |
| nucleoprotein, complete cds | | | | 06 | | |
| Influenza C virus (C/Mississippi/80) NP gene | 50.1 | 50.1 | 100% | 6.00E− | 100 | LC123735.1 |
| for nucleoprotein, complete cds | | | | 06 | | |
| Influenza C virus (C/Shizuoka/79) NP gene | 50.1 | 50.1 | 100% | 6.00E− | 100 | LC123734.1 |
| for nucleoprotein, complete cds | | | | 06 | | |
| Influenza C virus (C/Kyoto/1/79) NP gene for | 50.1 | 50.1 | 100% | 6.00E− | 100 | LC123733.1 |
| nucleoprotein, complete cds | | | | 06 | | |
| Influenza C virus (C/Kansas/1/79) NP gene | 50.1 | 50.1 | 100% | 6.00E− | 100 | LC123732.1 |
| for nucleoprotein, complete cds | | | | 06 | | |
| Influenza C virus (C/California/78) NP gene | 50.1 | 50.1 | 100% | 6.00E− | 100 | LC123731.1 |
| for nucleoprotein, complete cds | | | | 06 | | |
| Influenza C virus (C/Miyagi/77) NP gene for | 50.1 | 50.1 | 100% | 6.00E− | 100 | LC123730.1 |
| nucleoprotein, complete cds | | | | 06 | | |
| Influenza C virus (C/Kanagawa/1/76) NP | 50.1 | 50.1 | 100% | 6.00E− | 100 | LC123729.1 |
| gene for nucleoprotein, complete cds | | | | 06 | | |
| Influenza C virus (C/Aomori/74) NP gene for | 50.1 | 50.1 | 100% | 6.00E− | 100 | LC123728.1 |
| nucleoprotein, complete cds | | | | 06 | | |
| Influenza C virus (C/Sapporo/71) NP gene | 50.1 | 50.1 | 100% | 6.00E− | 100 | LC123727.1 |
| for nucleoprotein, complete cds | | | | 06 | | |
| Influenza C virus (C/Paris/1/67) NP gene for | 50.1 | 50.1 | 100% | 6.00E− | 100 | LC123725.1 |
| nucleoprotein, complete cds | | | | 06 | | |
| Influenza C virus (C/Yamagata/64) NP gene | 50.1 | 50.1 | 100% | 6.00E− | 100 | LC123724.1 |
| for nucleoprotein, complete cds | | | | 06 | | |
| Influenza C virus (C/Great Lakes/1167/1954) | 50.1 | 50.1 | 100% | 6.00E− | 100 | LC123723.1 |
| NP gene for nucleoprotein, complete cds | | | | 06 | | |
| Influenza C virus (C/Taylor/1233/1947) NP | 50.1 | 50.1 | 100% | 6.00E− | 100 | LC123722.1 |
| gene for nucleoprotein, complete cds | | | | 06 | | |
| Influenza C virus (C/India/P121719/2012) | 50.1 | 50.1 | 100% | 6.00E− | 100 | KM507549.1 |
| segment 5 nucleoprotein (NP) gene, partial | | | | 06 | | |
| cds | | | | | | |
| Influenza C virus (C/India/P135047/2013) | 50.1 | 50.1 | 100% | 6.00E− | 100 | KM507556.1 |
| segment 5 nucleoprotein (NP) gene, complete | | | | 06 | | |
| cds | | | | | | |
| Influenza C virus (C/Scotland/6855/2007) | 50.1 | 50.1 | 100% | 6.00E− | 100 | KT835360.1 |
| segment 5 nucleoprotein (NP) gene, partial | | | | 06 | | |
| cds | | | | | | |
| Influenza C virus (C/Scotland/5334/2007) | 50.1 | 50.1 | 100% | 6.00E− | 100 | KT835359.1 |
| segment 5 nucleoprotein (NP) gene, partial | | | | 06 | | |
| cds | | | | | | |
| Influenza C virus (C/Scotland/7274/2007) | 50.1 | 50.1 | 100% | 6.00E− | 100 | KT835358.1 |
| segment 5 nucleoprotein (NP) gene, partial | | | | 06 | | |
| cds | | | | | | |
| Influenza C virus (C/Scotland/7482/2007) | 50.1 | 50.1 | 100% | 6.00E− | 100 | KT835357.1 |
| segment 5 nucleoprotein (NP) gene, partial | | | | 06 | | |
| cds | | | | | | |
| Influenza C virus (C/Scotland/7383/2007) | 50.1 | 50.1 | 100% | 6.00E− | 100 | KT835356.1 |
| segment 5 nucleoprotein (NP) gene, partial | | | | 06 | | |
| cds | | | | | | |
| Influenza C virus (C/Scotland/5500/2007) | 50.1 | 50.1 | 100% | 6.00E− | 100 | KT835355.1 |
| segment 5 nucleoprotein (NP) gene, partial | | | | 06 | | |
| cds | | | | | | |
| Influenza C virus (C/Victoria/2/2012) | 50.1 | 50.1 | 100% | 6.00E− | 100 | KM504281.1 |
| segment 5 nucleoprotein gene, complete cds | | | | 06 | | |
| Influenza C virus (C/Singapore/DSO- | 50.1 | 50.1 | 100% | 6.00E− | 100 | JX963113.1 |
| 070203/2006) segment 5 nucleoprotein (NP) | | | | 06 | | |
| gene, partial cds | | | | | | |
| Influenza C virus (C/Singapore/DSO- | 50.1 | 50.1 | 100% | 6.00E− | 100 | JX963112.1 |
| 070193/2006) segment 5 nucleoprotein (NP) | | | | 06 | | |
| gene, partial cds | | | | | | |
| Influenza C virus (C/Singapore/DSO- | 50.1 | 50.1 | 100% | 6.00E− | 100 | JX963111.1 |
| 070170/2006) segment 5 nucleoprotein (NP) | | | | 06 | | |
| gene, partial cds | | | | | | |
| Influenza C virus (C/Singapore/DSO- | 50.1 | 50.1 | 100% | 6.00E− | 100 | JX963110.1 |
| 050530/2006) segment 5 nucleoprotein (NP) | | | | 06 | | |
| gene, partial cds | | | | | | |
| Influenza C virus (C/Eastern | 50.1 | 50.1 | 100% | 6.00E− | 100 | JX947862.1 |
| India/1202/2011) segment 5 nucleoprotein | | | | 06 | | |
| (NP) gene, partial cds | | | | | | |
| Influenza C virus (C/Yamagata/2/2010) NP | 50.1 | 50.1 | 100% | 6.00E− | 100 | AB684316.1 |
| gene for nucleoprotein, complete cds | | | | 06 | | |
| Influenza C virus (C/Yamagata/1/2007) NP | 50.1 | 50.1 | 100% | 6.00E− | 100 | AB684315.1 |
| gene for nucleoprotein, complete cds | | | | 06 | | |
| Influenza C virus (C/Yamagata/3/2005) NP | 50.1 | 50.1 | 100% | 6.00E− | 100 | AB684314.1 |
| gene for nucleoprotein, complete cds | | | | 06 | | |
| Influenza C virus (C/Yamagata/15/2004) NP | 50.1 | 50.1 | 100% | 6.00E− | 100 | AB684313.1 |
| gene for nucleoprotein, complete cds | | | | 06 | | |
| Influenza C virus (C/Yamagata/3/2000) NP | 50.1 | 50.1 | 100% | 6.00E− | 100 | AB684311.1 |
| gene for nucleoprotein, complete cds | | | | 06 | | |

In some embodiments, the composition described herein comprises probe 8. In additional embodiments, probe 8 may hybridize to a target nucleotide molecule that is a part of Respiratory syncytial virus (RSV), causing RSV infection. In some embodiments, probe 8 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 8. In some embodiments, probe 8 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 8.

In some embodiments, the composition described herein comprises probe 9. In additional embodiments, probe 9 may hybridize to a target nucleotide molecule that is a part of SARS-CoV-2, a novel coronavirus causing Coronavirus disease 2019 (COVID-19). In some embodiments, probe 9 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 9. In some embodiments, probe 9 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 9.

In some embodiments, the composition described herein comprises probe 10. In additional embodiments, probe 10 may hybridize to a target nucleotide molecule that is a part of SARS-CoV, a novel coronavirus causing Severe Acute Respiratory Syndrome (SARS). In some embodiments, probe 10 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 10. In some embodiments, probe 10 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 10.

In some embodiments, the composition described herein comprises probe 11. In additional embodiments, probe 11 may hybridize to a target nucleotide molecule that is a part of a housekeeping gene in RP. In some embodiments, probe 11 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 11. In some embodiments, probe 11 consists of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 11.

TABLE 4
Nucleic acid sequences of probes

| Probe | |
|---|---|
| number | Nucleic Acid Sequence |
| Probe 1 | GCTGCCCAAACGATGGCTGAAAAAGGGAGGCAGC (SEQ ID NO: 1) |
| Probe 2 | GCTGCCAATCGCAAAAGCTGTTCAGTCACGGCAGC (SEQ ID NO: |
| | 2) |
| Probe 3 | CGCGCTACCCAGTCATAACTTACTCAACAGCAACAGCGCG (SEQ |
| | ID NO: 3) |
| Probe 4 | GCTGCCGTCTCAAAATTTGTTGATCAAGACAATACAATTGGCAGC |
| | (SEQ ID NO: 4) |
| Probe 5 | GCTGCCAGGGCTTTCACCGAAGAGGGGGCAGC (SEQ ID NO: 5) |
| Probe 6 | GCTGCCAACGAAGTAGGTGGAGACGGAGGGGCAGC (SEQ ID NO: |
| | 6) |
| Probe 7 | TTGCAAATTGGCTTGCCCCCACT (SEQ ID NO: 7) |
| Probe 8 | GCGAGCCCATGTGAATTCCCTGCATCAATGCTCGC (SEQ ID NO: 8) |
| Probe 9 | ACCCCGCATTACGTTTGGTGGACC (SEQ ID NO: 9) |
| Probe 10 | AYCACATTGGCACCCGCAATCCTG (SEQ ID NO: 10) |
| Probe 11 | TTCTGACCTGAAGGCTCTGCGCG (SEQ ID NO: 11) |

In some embodiments, the composition further comprises at least one forward primer selected from the group consisting of forward primers comprising a nucleic acid sequence having at least 85% sequence identity to SEQ ID NO: 12, 13, 14, 15, 16, 17, 18, 19, 20, 21, or 22. In some embodiments, the composition further comprises at least one reverse primer selected from the group consisting of reverse primers comprising a nucleic acid sequence having at least 85% sequence identity to SEQ ID NO: 23, 24, 25, 26, 27, 28, 29, 30, 31, 32, or 33. In additional embodiments, the composition described herein comprises at least one: (a) forward primer selected from the group consisting of forward primers comprising a nucleic acid sequence having at least 85% sequence identity to SEQ ID NO: 12, 13, 14, 15, 16, 17, 18, 19, 20, 21, or 22; and (b) reverse primer selected from the group consisting of reverse primers comprising a nucleic acid sequence having at least 85% sequence identity to SEQ ID NO: 23, 24, 25, 26, 27, 28, 29, 30, 31, 32, or 33.

In some embodiments, the composition further comprises at least one forward primer selected from the group consisting of forward primers comprising a nucleic acid sequence having at least 100% sequence identity to SEQ ID NO: 12, 13, 14, 15, 16, 17, 18, 19, 20, 21, or 22. In some embodiments, the composition further comprises at least one reverse primer selected from the group consisting of reverse primers comprising a nucleic acid sequence having at least 100% sequence identity to SEQ ID NO: 23, 24, 25, 26, 27, 28, 29, 30, 31, 32, or 33. In additional embodiments, the composition described herein comprises at least one: (a) forward primer selected from the group consisting of forward primers comprising a nucleic acid sequence having at least 100% sequence identity to SEQ ID NO: 12, 13, 14, 15, 16, 17, 18, 19, 20, 21, or 22; and (b) reverse primer selected from the group consisting of reverse primers comprising a nucleic acid sequence having at least 100% sequence identity to SEQ ID NO: 23, 24, 25, 26, 27, 28, 29, 30, 31, 32, or 33.

In some embodiments, the composition further comprises at least one forward primer selected from the group consisting of forward primers comprising a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 12, 13, 14, 15, 16, 17, 18, 19, 20, 21, or 22. In some embodiments, the composition further comprises at least one reverse primer selected from the group consisting of reverse primers comprising a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 23, 24, 25, 26, 27, 28, 29, 30, 31, 32, or 33. In additional embodiments, the composition described herein comprises at least one: (a) forward primer selected from the group consisting of forward primers comprising a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 12, 13, 14, 15, 16, 17, 18, 19, 20, 21, or 22; and (b) reverse primer selected from the group consisting of reverse primers comprising a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 23, 24, 25, 26, 27, 28, 29, 30, 31, 32, or 33.

In some embodiments, the composition further comprises at least one forward primer selected from the group consisting of forward primers consisting of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 12, 13, 14, 15, 16, 17, 18, 19, 20, 21, or 22. In some embodiments, the composition further comprises at least one reverse primer selected from the group consisting of reverse primers consisting of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 23, 24, 25, 26, 27, 28, 29, 30, 31, 32, or 33. In additional embodiments, the composition described herein comprises at least one: (a) forward primer selected from the group consisting of forward primers consisting of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 12, 13, 14, 15, 16, 17, 18, 19, 20, 21, or 22; and (b) reverse primer selected from the group consisting of reverse primers consisting of a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 23, 24, 25, 26, 27, 28, 29, 30, 31, 32, or 33.

In some embodiments, the composition described herein comprises at least one forward primer selected from the group consisting of forward primer 1, forward primer 2, forward primer 3, forward primer 4, forward primer 5, forward primer 6, forward primer 7, forward primer 8, forward primer 9, forward primer 10, and forward primer 11. In some embodiments, the composition described herein comprises at least one reverse primer selected from the group consisting of reverse primer 1, reverse primer 2, reverse primer 3, reverse primer 4, reverse primer 5, reverse primer 6, reverse primer 7, reverse primer 8, reverse primer 9, reverse primer 10, and reverse primer 11.

In some embodiments, the composition described herein comprises at least forward primer 1, reverse primer 1, and probe 1, wherein forward primer 1 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 12, wherein reverse primer 1 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 23, and wherein probe 1 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 1.

In some embodiments, the composition described herein comprises at least forward primer 2, reverse primer 2, and probe 2, wherein forward primer 2 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 13, wherein reverse primer 2 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 24, and wherein probe 2 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 2.

In some embodiments, the composition described herein comprises at least forward primer 3, reverse primer 3, and probe 3, wherein forward primer 3 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 14, wherein reverse primer 3 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 25, and wherein probe 3 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 3.

In some embodiments, the composition described herein comprises at least forward primer 4, reverse primer 4, and probe 4, wherein forward primer 4 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 15, wherein reverse primer 4 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 26, and wherein probe 4 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 4.

In some embodiments, the composition described herein comprises at least forward primer 5, reverse primer 5, and probe 5, wherein forward primer 5 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 16, wherein reverse primer 5 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 27, and wherein probe 5 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 5.

In some embodiments, the composition described herein comprises at least forward primer 6, reverse primer 6, and probe 6, wherein forward primer 6 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 17, wherein reverse primer 6 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 28, and wherein probe 6 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 6.

In some embodiments, the composition described herein comprises at least forward primer 7, reverse primer 7, and probe 7, wherein forward primer 7 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 18, wherein reverse primer 7 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 29, and wherein probe 7 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 7.

In some embodiments, the composition described herein comprises at least forward primer 8, reverse primer 8, and probe 8, wherein forward primer 8 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 19, wherein reverse primer 8 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 30, and wherein probe 8 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 8.

In some embodiments, the composition described herein comprises at least forward primer 9, reverse primer 9, and probe 9, wherein forward primer 9 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 20, wherein reverse primer 9 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 31, and wherein probe 9 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 9.

In some embodiments, the composition described herein comprises at least forward primer 10, reverse primer 10, and probe 10, wherein forward primer 10 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 21, wherein reverse primer 10 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 32, and wherein probe 10 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 10.

In some embodiments, the composition described herein comprises at least forward primer 11, reverse primer 11, and probe 11, wherein forward primer 11 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 22, wherein reverse primer 11 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 33, and wherein probe 11 comprises a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 11.

In some embodiments, the composition described herein comprises at least forward primers 1, 2, 3, and 4, reverse primers 1, 2, 3, and 4, and probes 1, 2, 3, and 4, wherein forward primers 1, 2, 3, and 4 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 12, 13, 14, and 15, respectively, wherein reverse primers 1, 2, 3, and 4 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 23, 24, 25, and 26, respectively, and wherein probes 1, 2, 3, and 4 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 1, 2, 3, and 4.

In some embodiments, the composition described herein comprises at least forward primers 7, 9, and 10, reverse primers 7, 9, and 10, and probes 7, 9, and 10, wherein forward primers 7, 9, and 10 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 18, 20, and 21, respectively, wherein reverse primers 7, 9, and 10 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 29, 31, and 32, respectively, and wherein probes 7, 9, and 10 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 7, 9, and 10.

In some embodiments, the composition described herein comprises at least forward primers 5, 6, and 8, reverse primers 5, 6, and 8, and probes 5, 6, and 8, wherein forward primers 5, 6, and 8 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 16, 17, and 19, respectively, wherein reverse primers 5, 6, and 8 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 27, 28, and 30, respectively, and wherein probes 5, 6, and 8 comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 5, 6, and 8.

TABLE 5
Nucleic acid sequences of forward primers and reverse primers

| Primer | Nucleic Acid Sequence | Nucleic Acid Sequence |
|---|---|---|
| number | of forward primer | of reverse primer |
| 1 | ACCTACAAGGCAACAACATC | CTTCCTGCTGGTGTGTTAAT |
| | (SEQ ID NO: 12) | (SEQ ID NO: 23) |
| 2 | CCATTTACCTAAGTGATGGAA | CGTGGCATAATCTTCTTTTT |
| | (SEQ ID NO: 13) | (SEQ ID NO: 24) |
| 3 | GGAGCATTGTGTCATCTGTC | TAGTGTGTAATGCAGCTCGT |
| | (SEQ ID NO: 14) | (SEQ ID NO: 25) |
| 4 | CCTGGAGTCCCATCAAAAGT | GCATCTATACGAACACCTGCT |
| | (SEQ ID NO: 15) | (SEQ ID NO: 26) |
| 5 | AAAGCGAATTTCAGTGTGAT | GAAGGCAATGGTGAGATTT |
| | (SEQ ID NO: 16) | (SEQ ID NO: 27) |
| 6 | GTCCATCAAGCTCCAGTTTT | TCTTCTTACAGCTTGCTTGC |
| | (SEQ ID NO: 17) | (SEQ ID NO: 28) |
| 7 | CCACTACTCCCATTTCGTCAG | CAGTATGTGTAGTGCGCATATAA |
| | (SEQ ID NO: 18) | GCA (SEQ ID NO: 29) |
| 8 | TTTCCACAATATYTAAGTGTCAA | TCATCWCCATACTTTTCTGTTA |
| | (SEQ ID NO: 19) | (SEQ ID NO: 30) |
| 9 | GACCCCAAAATCAGCGAAAT | TCTGGTTACTGCCAGTTGAATCT |
| | (SEQ ID NO: 20) | G (SEQ ID NO: 31) |
| 10 | GGGAGCCTTGAATACACCAAAA | TGTAGCACGATTGCAGCATTG |
| | (SEQ ID NO: 21) | (SEQ ID NO: 32) |
| 11 | AGATTTGGACCTGCGAGCG (SEQ | GAGCGGCTGTCTCCACAAGT |
| | ID NO: 22) | (SEQ ID NO: 33) |

In some embodiments, the composition further comprises at least one dye selected from the group consisting of JUN-QSY, ABY-QSY, FAM, VIC, and Cy5. In additional embodiments, the at least one dye is JUN-QSY. In additional embodiments, the at least one dye is ABY-QSY. In additional embodiments, the at least one dye is FAM. In additional embodiments, the at least one dye is VIC. In additional embodiments, the at least one dye is Cy5.

### III. METHODS

The present disclosure relates to methods and compositions for detecting and treating respiratory illness. The methods and compositions described herein comprise at least one probe that may hybridize with a target nucleotide molecule and may further comprise at least one forward primer, at least one reverse primer, and/or at least one dye.

In one aspect, the present disclosure relates to a method for detecting a respiratory illness, comprising: hybridizing a target nucleotide molecule in a sample with a composition comprising at least one probe selected from the group consisting of probe 1, probe 2, probe 3, probe 4, probe 5, probe 6, probe 7, probe 8, probe 9, probe 10, and probe 11, wherein probe 1 comprises a nucleic acid sequence having at least 85% sequence identity to SEQ ID NO: 1, wherein probe 2 comprises a nucleic acid sequence having at least 85% sequence identity to SEQ ID NO: 2, wherein probe 3 comprises a nucleic acid sequence having at least 85% sequence identity to SEQ ID NO: 3, wherein probe 4 comprises a nucleic acid sequence having at least 85% sequence identity to SEQ ID NO: 4, wherein probe 5 comprises a nucleic acid sequence having at least 85% sequence identity to SEQ ID NO: 5, wherein probe 6 comprises a nucleic acid sequence having at least 85% sequence identity to SEQ ID NO: 6, wherein probe 7 comprises a nucleic acid sequence having at least 85% sequence identity to SEQ ID NO: 7, wherein probe 8 comprises a nucleic acid sequence having at least 85% sequence identity to SEQ ID NO: 8, wherein probe 9 comprises a nucleic acid sequence having at least 85% sequence identity to SEQ ID NO: 9, wherein probe 10 comprises a nucleic acid sequence having at least 85% sequence identity to SEQ ID NO: 10, wherein probe 11 comprises a nucleic acid sequence having at least 85% sequence identity to SEQ ID NO: 11.

In another aspect, the present disclosure relates to a method for treating a respiratory illness, comprising detecting the respiratory illness according to a method described herein.

In some embodiments, the respiratory illness is selected from the group consisting of Parainfluenza 1, Parainfluenza 2, Parainfluenza 3, Parainfluenza 4, Influenza A, Influenza B, MERS, RSV infection, COVID-19, and SARS. In additional embodiments, the respiratory illness is Parainfluenza 1. In additional embodiments, the respiratory illness is Parainfluenza 2. In additional embodiments, the respiratory illness is Parainfluenza 3. In additional embodiments, the respiratory illness is Parainfluenza 4. In additional embodiments, the respiratory illness is Influenza A. In additional embodiments, the respiratory illness is Influenza B. In additional embodiments, the respiratory illness is MERS. In additional embodiments, the respiratory illness is RSV infection. In additional embodiments, the respiratory illness is COVID-19. In additional embodiments, the respiratory illness is SARS.

In additional embodiments, the respiratory illness is MERS, COVID-19, and SARS. In additional embodiments, the respiratory illness is Influenza A, Influenza B, and RSV infection. In additional embodiments, the respiratory illness is Parainfluenza 1, Parainfluenza 2, Parainfluenza 3, and Parainfluenza 4.

In some embodiments, the method described herein comprises a sample obtained from a bodily fluid of a subject. In some embodiments, the bodily fluid is saliva, a nasopharyngeal fluid, an oropharyngeal fluid, a mid-turbinate fluid, or blood. In additional embodiments, the bodily fluid is saliva. In additional embodiments, the bodily fluid is a nasopharyngeal fluid. In additional embodiments, the bodily fluid is an oropharyngeal fluid. In additional embodiments, the bodily fluid is a mid-turbinate fluid. In additional embodiments, the bodily fluid is blood. In additional embodiments, the subject is human.

In some embodiments, the method described herein further comprises obtaining a sample from a subject comprising drawing blood from the subject. In some embodiments, the method described herein further comprises obtaining a sample from a subject comprising administering a swab to a cavity of the subject. In some embodiments, the cavity is a nostril or mouth of the subject.

In one aspect, the present disclosure relates to a method for detecting a respiratory illness, comprising: Real-Time Reverse Transcriptase (RT)-PCR assays for the in vitro qualitative detection of nucleic acid from Influenza A virus, Influenza B virus, and Influenza C virus in nasopharyngeal, oropharyngeal, and mid-turbinate swab samples.

### A. RNA Extraction by Genolution NexTractor NX-48S Automated RNA Extraction Instrument

In some embodiments, the method comprises using a Genolution Nextractor NX-48S, which is an automatic extraction system, for rapid isolation of RNA from a variety of samples. In some embodiments, using the Nextractor purifies up to 48 samples at one time and also offers a high throughput processing and yield. In some embodiments, the Nextractor is a fully automated walk away system and the reagent cartridges are pre-filled.

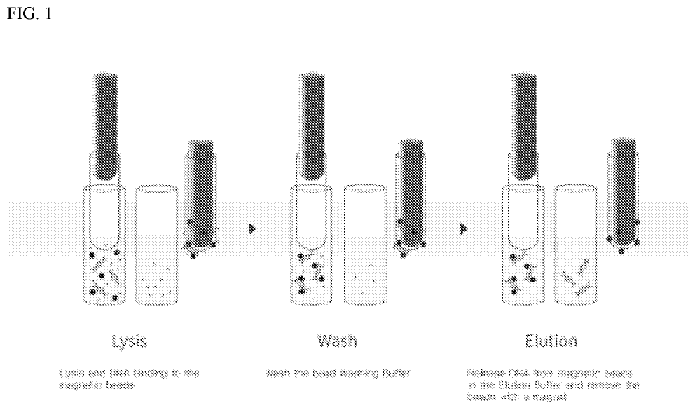

FIG. 1 depicts The principle of nucleic acid extraction from a sample using the magnetic beads method is shown in FIG. 1. In some embodiments, the extraction comprises: (a) lysis, wherein the sample is lysed and DNA is bound to magnetic beads; (b) wash, wherein the beads are washed with washing buffer; and (c) elution, wherein DNA is released from magnetic beads in elution buffer and the beads removed with a magnet.

### B. Real-Time RT PCR

In some embodiments, the method comprises utilizing a fluorescent reporter probe, which has proven to be the most accurate and most reliable of methods. In some embodiments, the method comprises using a sequence-specific DNA-based probe to quantify only the DNA containing the probe sequence. In some embodiments, use of the reporter probe significantly increases specificity and allows quantification even in the presence of non-specific DNA amplification.

In some embodiments, fluorescence is detected and measured in a real-time PCR thermal cycler, and its geometric increase corresponding to exponential increase of the product is used to determine the ΔRn in each reaction.

In some embodiments, the method comprises a nuclease assay. In some embodiments, extracted RNA is introduced into a reaction mixture consisting of Master Mix, forward and reverse primers, and probes. In some embodiments, fluorescence dye probe anneals specifically to a complementary sequence, if present, between the forward and reverse primers sites. In some embodiments, reverse Real-Time PCR (rRT-PCR) is one of the most powerful and sensitive gene analysis techniques available. In some embodiments, rRT-PCR is used for a broad range of applications, including quantitative gene expression analysis, genotyping, copy number, drug target validation, biomarker discovery, pathogen detection, and measuring RNA interference.

In some embodiments, every rRT-PCR contains a fluorescent reporter molecule, a TaqMan® probe to monitor the accumulation of PCR product. In some embodiments, as the quantity of target amplicon increases, so does the amount of fluorescence emited from the fluorophore.

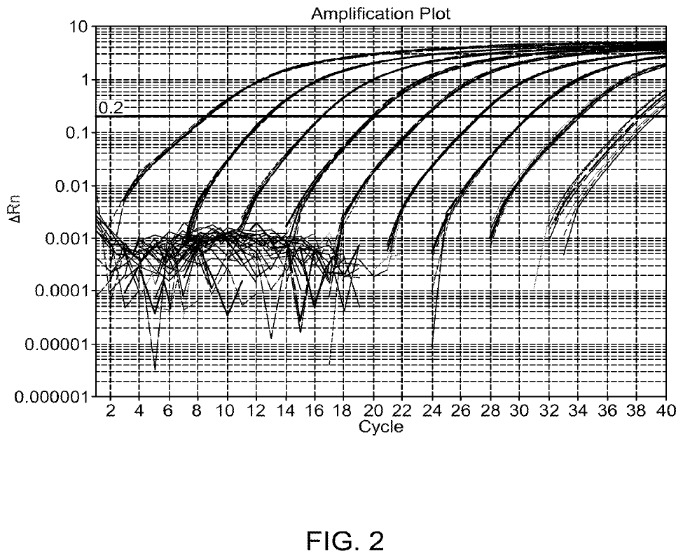

Exponential phase measurement in Real-Time PCR is shown in FIG. 2. In some embodiments, Real-Time PCR focuses on the exponential phase, which provides the most precise and accurate data for quantitation. In some embodiments, during the exponential phase, the real-time PCR instrument calculates two values: (a) Threshold: the level of detection at which a reaction reaches a fluorescent intensity above background; and (b) Ct: the PCR cycle at which the sample reaches the threshold, wherein the Ct value is used in absolute or relative quantitation.

### IV. VALIDATION PROCEDURE

In one aspect, the present disclosure relates to a method for detecting a respiratory illness, comprising: Real-Time Reverse Transcriptase (RT)-PCR assays for the in vitro qualitative detection of nucleic acids, which requires a validation procedure.

In some embodiments, the method comprises following a technical procedure for validation.

In some embodiments, the method comprises a validation design chart comprising: (a) RNA Extraction by Genolution Nextractor NX-48S; (b) PCR Amplification performed on a 7500 Fast Real-Time PCR System instrument; (c) all RNA extractions and PCR test runs documented in batch records, wherein documentation includes date of run, operator name, instrument name, reagent lot number, reagent expiration date, sample name and position on PCR plate map.

In some embodiments, RNA Extraction Materials and Equipment are used as shown in Table 6.

TABLE 6
RNA Extraction Materials and Equipment

| RNA | | | |
|---|---|---|---|
| EXTRACTION | | CATALOG | |
| KIT | SUPPLIER | NUMBER | STORAGE |
| Genolution RNA | Genolution | VN144 | 15° C. to 25° C. |
| Extraction Kit | | | |

RNA EXTRACTION KIT

| COMPONENTS | QUANTITY |
|---|---|
| Number of Samples per Kit | 48 |
| Prefilled extraction block | 2 |
| Magnetic Beads | 1 bottle |

In some embodiments, real-time RT-PCR materials and equipment are used as shown in Table 7.

TABLE 7
Real-Time RT-PCR Materials and Equipment

| | | CATALOG | STORAGE |
|---|---|---|---|
| PCR REAGENT | SUPPLIER | NUMBER | CONDITION |
| TaqPath ™ 1-Step | ThermoFisher | A28522 | −15° C. to −25° C. |
| Multiplex Master | | | |
| Mix (No ROX) | | | |
| TaqPath ™ 1-Step | ThermoFisher | A28527 | −15° C. to −25° C. |
| Multiplex Master | | | |
| Mix | | | |
| Influenza A Forward | Avellino | N/A | −15° C. to −25° C. |
| Primer | | | |
| Influenza A Reverse | Avellino | N/A | −15° C. to −25° C. |
| Primer | | | |
| Influenza A Probe | Avellino | N/A | −15° C. to −25° C. |
| Influenza B Forward | Avellino | N/A | −15° C. to −25° C. |
| Primer | | | |
| Influenza B Reverse | Avellino | N/A | −15° C. to −25° C. |
| Primer | | | |
| Influenza B Probe | Avellino | N/A | −15° C. to −25° C. |
| Influenza C Forward | Avellino | N/A | −15° C. to −25° C. |
| Primer | | | |
| Influenza C Reverse | Avellino | N/A | −15° C. to −25° C. |
| Primer | | | |
| Influenza C Probe | Avellino | N/A | −15° C. to −25° C. |

In some embodiments, equipment and suppliers are used as shown in Table 8.

TABLE 8
Equipment and suppliers

| EQUIPMENT | SUPPLIER |
|---|---|
| 7500 Fast Real-Time PCR System | Life Technologies |
| PCR Plate Spinner or equivalent | VWR |

In some embodiments, general material and equipment are used as shown in Table 9.

TABLE 9
general material and equipment

| GENERAL MATERIAL | | CATALOG |
|---|---|---|
| and EQUIPMENT | SUPPLIER | NUMBER |
| MicroAmp ™ Optical 96- | Life Technologies | 4346906 |
| Well Reaction Plate with | | |
| Barcode | | |
| Splash-free Support Base | Life Technologies | 4312063 |
| MicroAmp ™ Adhesive Film | Life Technologies | 4333183 |
| Applicator | | |
| MicroAmp ™ Optical | Life Technologies | 4311971 |
| Adhesive Film | | |
| LSE Mini Microcentrifuge | Corning | 6765 |
| or equivalent | | |
| Vortexer (Capsule) or | VWR | G-560 |
| equivalent | | |
| Single Channel Pipettes | Rainin | LTS 1000 uL |
| | | LTS 200 uL |
| | | LTS 20 uL |
| | | LTS 10 uL |
| | | LTS 2 uL |
| Multi Channel Pipettes | Rainin | LA6-1200XLS |
| | | LTS 1200 uL |
| | | LTS 300 uL |
| | | LTS 10 uL |
| Single Channel Repeater | Fisher Scientific | Biohit Epet |
| Pipettes | | BE-100 |
| Pipette tips, aerosol resistant | Rainin | RT-L10F |
| or equivalent | | RT-L200F |
| | | RT-L300F |
| | | RT-L1000F |
| | Fisher Scientific | 02-707-431 |
| Water, UltraPure Distilled | Invitrogen | 10977-023 |
| Water, Protease Free or | | |
| equivalent | | |
| Disposable gloves and other | Fisher Scientific | EV-2050 |
| appropriate PPEs | | (Gloves) |
| | | 19-460-102 |
| | | (Face shields) |
| | | 10837-036 |
| | | (Disposable |
| | | Gowns) |
| Buccal Swabs | Copan | 155C |
| (for sample collection) | | |
| Microsoft Excel ® | Microsoft Office 2010 | N/A |
| spreadsheet or equivalent | | |
| Analysis Software | ABI 7500 Software V2.3 | N/A |
| 50 mL Serological Pipettes | Fisher Scientific | 13-675-27 |
| 10 mL Serological Pipettes | Fisher Scientific | 13-675-20 |
| 25 mL Serological Pipettes | Fisher Scientific | 13-688-2 |
| 100 mL Serological Pipettes | Fisher Scientific | 13-675-22 |
| Disposable Transfer Pipettes | Fisher Scientific | 13-711-9D |
| AccuHelp Motorized Pipette | AccuPet | PH01-G |
| Controller or equivalent | | |

In some embodiments, the method comprises quality controls comprising: (a) assay controls run concurrently with all test samples; (b) PTC—Positive Template Control with an expected Ct value range; (c) NTC—No Template Control; (d) EC—Extraction Control extracted concurrently with the test samples, providing a nucleic acid extraction procedural control and a secondary negative control that validates the nucleic extraction procedure and reagent integrity; and (e) RP—all clinical samples tested for human RNAse P (RNP) gene to assess specimen quality.

In some embodiments, the method comprises an rRNA Extraction and Quantification Procedure. In some embodiments, the method comprises an Automated Extraction Procedure.

In some embodiments, the method comprises a sample obtained from starting material comprising: nasopharyngeal, oropharyngeal and mid-turbinate cells collected on buccal swabs from individuals or subjects, wherein nucleic acid is extracted from the collected buccal swabs for use in validation. In some embodiments, yield and quality of the purified nucleic acid depend on the storage conditions of the swab. In some embodiments, fresher samples collected with buccal swabs yield better results.

In some embodiments, yield and quality of purified nucleic acid are determined by various factors. In some embodiments, nucleic acid yields depend on the number of epithelial cells present. In some embodiments, yields from buccal swabs differ by as much as tenfold. In some embodiments, yield and quality of the purified nucleic Acid are determined by a precision and accuracy evaluation of multiple measurements of each analyte.

In some embodiments, the method comprises a Real-Time RT-PCR Plate Set Up Procedure. In some embodiments, Extracted Nucleic Acid (RNA) from the validation samples is used for analysis. In some embodiments, the following general PCR precautions are taken to avoid contamination: (a) clean lab coat and clean gloves are worn when preparing samples for PCR amplification; (b) gloves or lab coat are changed whenever contamination is suspected; (c) separate areas and dedicated equipment and supplies for each of the following procedures are maintained: Sample preparation, PCR setup, PCR amplification, and analysis of PCR products; (d) amplified PCR products are never brought into the PCR setup area; (e) all sample tubes are carefully opened and closed to prevent potential splash or spray of PCR samples; (f) reactions and components are kept covered or capped as much as possible; (g) plates are kept covered or capped between the PCR runs; (h) a positive-displacement pipette or aerosol-resistant pipette tips are used; (i) lab benches and equipment are cleaned before and after each procedure.

In some embodiments, the method comprises PCR Reagents and Storage as follows: (a) all TaqMan reagents are kept protected from light until they are needed for use, wherein excessive exposure to light may affect the fluorescent probes. (b) freeze-thaw cycles are minimized; (c) prior to use, the PCR Mixes are allowed to thaw at 2° C. to 8° C. for use at a later time within the day, wherein if immediate use is required, the PCR Mixes may be thawed at room temperature; (d) when thawed, the reagents are suspended by flicking the tubes or by giving them a light vortex, and then centrifuging the tubes briefly; (e) Primer, Probe and materials used for their creation, are stored at −25° C. to −15° C.; (f) TaqPath™ 1-Step RT-qPCR Master Mix is stored at −25° C. to −15° C.

In some embodiments, the method comprises preparing PCR master mixes according to the volumes shown in Table 10.

TABLE 10
PCR master mix preparation

| | | Vol. of Reagent |
|---|---|---|
| Step # | Reagent | Added per Reaction |
| 1 | Nuclease-free Water | N × 6 μL |
| 2 | Influenza A PCR Mix | N × 1 μL |
| | (Primer & Probe Set) | |
| 3 | Influenza B PCR Mix | N × 2 μL |
| | (Primer & Probe Set) | |
| 4 | Influenza C PCR Mix | N × 1 μL |
| | (Primer & Probe Set) | |
| 5 | TaqPath ™ 1-Step RT-qPCR | N × 5 μL |
| | Master Mix | |
| | Total Volume | 15 l |

In some embodiments, the method comprises a MicroAmp™ Optical 96-Well Reaction Plate Set Up comprising Master Mix Addition. In some embodiments, when the reaction plate is outside the instrument, the reaction plate is placed in a splash-free support base to avoid placing the plate onto a contaminated surface that could cause optical contamination.

In some embodiments, the method comprises a Manual PCR Set-up comprising: (a) Pipetting 15.0 μL of Master Mix to each well of the microplate with the use of an automatic pipettor, wherein the NTC for PCR should be the first well that receives Master Mix; (b) into each well of the plate, pipetting 5.0 μL of controls, NTCs, and extracted RNA samples onto the side of the well with the use of a single channel pipette; (c) covering the plate with MicroAmp Optical Adhesive Film; (d) inspecting each well for volume uniformity, noting any wells that do not contain the proper volume; (e) centrifuging the plate for 15 to 20 seconds in the PCR Plate Spinner to spin down the contents, wherein the PCR Plate Spinner automatically accelerates to 2,500 RPM; and (f) visually checking contents of wells to ensure the mix is at the bottom, repeatping spinning if necessary.

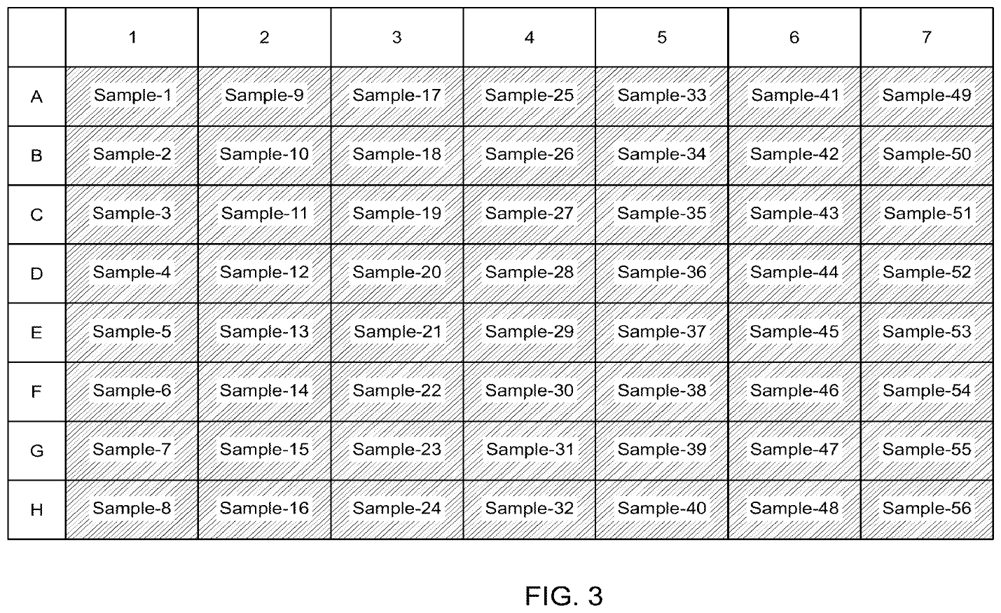

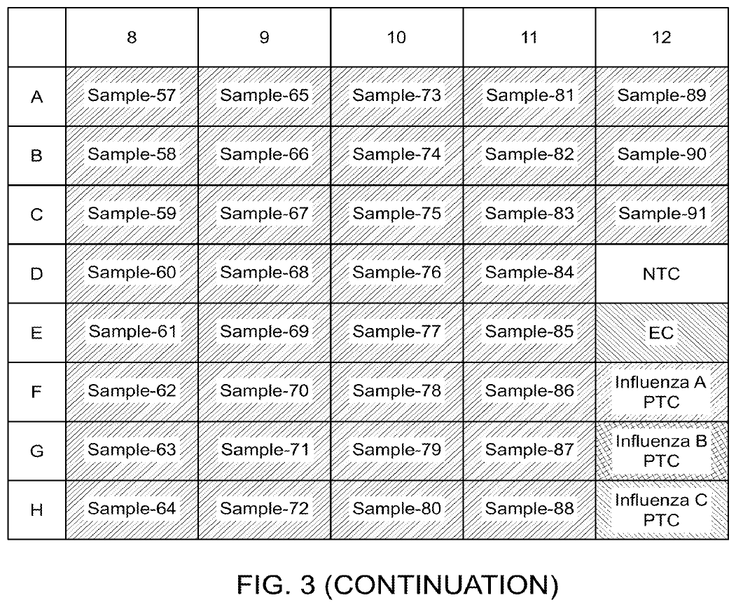

An example of a PCR Plate Map, showing placement for extracted RNA samples, controls, and NTCs, is shown in FIG. 3.

In some embodiments, the method comprises performing PCR using the 7500 Fast Real-Time PCR System Instrument. In some embodiments, PCR is performed on each plate on 7500 Fast Real-Time PCR System (7500 Fast) instruments using the following cycling conditions: instrument Settings as shown in Table 11: Detector (FAM, JUN & NED); Quencher (None); Passive Reference: (None or MUSTANG PURPLE); Run Mode: (Fast); Sample Volume (20 μL).

TABLE 11
Instrument settings

| Step | Cycles | Temp | Time |
|---|---|---|---|
| UNG Incubation | 1 | 25° C. | 2 min |
| RT Incubation | 1 | 50° C. | 15 min |
| Enzyme Activation | 1 | 95° C. | 2 min |
| Amplification | 40 | 95° C. | 3 sec |
| | | 55° C. | 30 sec |

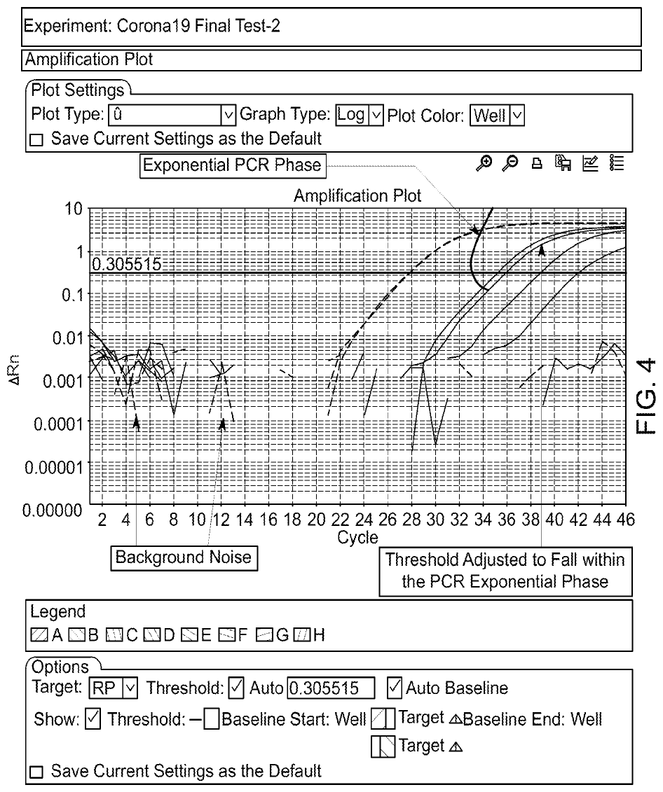

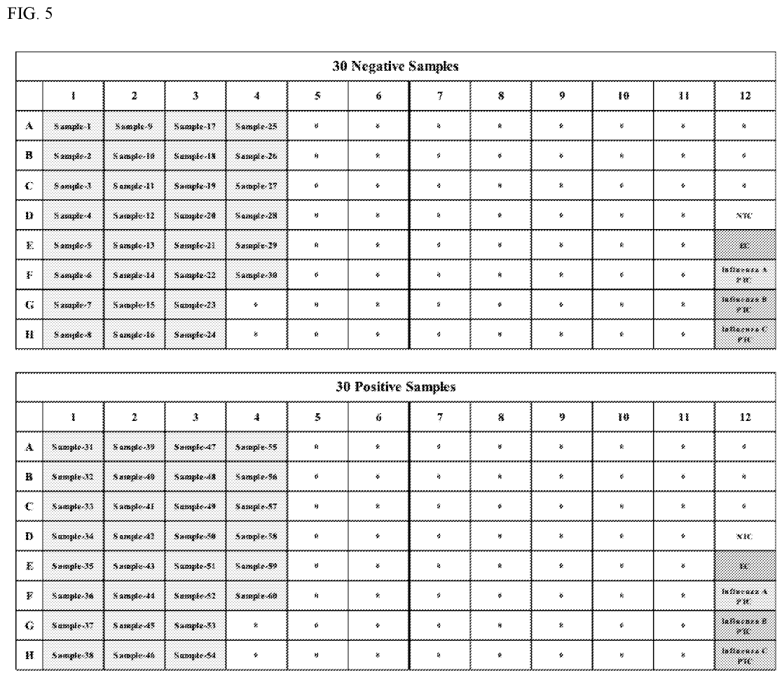

In some embodiments, the method comprises analysis after PCR amplification. In some embodiments, after completion of the run, the data are saved and analyzed following the instrument manufacturer's instructions. In some embodiments, analyses are performed separately for each target using a manual threshold setting. In some embodiments, thresholds are adjusted to fall within exponential phase of the fluorescence curves and above any background signal (FIG. 4). In some embodiments, the procedure chosen for setting the threshold is used consistently.

In some embodiments, the method comprises interpreting test results. In some embodiments, NTCs are negative and do not exhibit fluorescence growth curves that cross the threshold line. In some embodiments, if a false positive occurs with one or more of the primer and probe NTC reactions, sample contamination may have occurred, in which case the run is invalidated and the assay repeated with stricter adherence to the procedure guidelines.

In some embodiments, PTC reaction produces a positive result with an expected Ct value for each target included in the test. In some embodiments, if expected positive reactivity is not achieved, the run is invalidated and the assay repeated with stricter adherence to procedure guidelines, in which case the cause of failed PTC reactivity is determined, corrective actions implemented, and results of the investigation and corrective actions documented. In some embodiments, PTC reagents that do not generate expected result are not used.

In some embodiments, RP is positive at or before 38 cycles for all clinical samples and EC, thus indicating the presence of sufficient nucleic acid from human RNase P gene and that the specimen is of acceptable quality. In some embodiments, failure to detect RNase P in EC may indicate: (1) improper assay set up and execution; and (2) Reagent or equipment malfunction. In some embodiments, detection of RNase P in EC but failure to detect RNase P in any of the clinical samples may indicate: (1) improper extraction of nucleic acid from clinical materials resulting in loss of nucleic acid or carry-over of PCR inhibitors from clinical specimens; and (2) absence of sufficient human cellular material in sample to enable detection.

In some embodiments, EC is negative for influenza specific primer/probe sets. In some embodiments, if any specific primer/probes exhibit a growth curve that crosses the threshold line, a specific interpretation is followed. In some embodiments, the specific interpretation comprises: (1) interpreting that contamination of nucleic acid extraction reagents occurred, in which case the run is invalidated and reagent integrity of nucleic acid extraction reagents confirmed prior to further testing; and (2) interpreting that cross contamination of samples occurred during nucleic acid extraction procedures or assay setup, in which case the run is invalidated and the assay repeated with stricter adherence to procedure guidelines.

In some embodimets, when all controls exhibit the expected performance, a specimen is considered negative if all markers cycle threshold growth curves do not cross the threshold and the RNase P growth curve does cross the threshold line.

In some embodiments, when all controls exhibit the expected performance, a specimen is considered positive for all markers cycle threshold growth curve crosses the threshold line, wherein the RNase P may or may not be positive as described, but the result is still valid.

In some embodiments, when all controls exhibit the expected performance and the growth curves for the markers and the RNase P marker do not cross the cycle threshold growth curve, the result is invalid, in which case the extracted RNA from the specimen is re-tested. In some embodiments, if residual RNA is not available, RNA is re-extracted from residual specimen and re-tested. In some embodiments, if the re-tested sample is negative for all markers and all controls exhibit the expected performance, the result is “Invalid.”

In some embodiments, when all controls exhibit the expected performance and the cycle threshold growth curve for any marker but not all cross the threshold line the result is inconclusive, in which case, RNA is re-extracted from residual specimen and re-tested. Table 12 shows how test results are interpreted.

TABLE 12
Test Results Interpretation

| | | Result | Expected |
|---|---|---|---|
| Target | Results | Interpretation | Ct Value |
| Influenza A | +/− | Detected/Not | <40 Ct |
| | | Detected | |
| Influenza B | +/− | Detected/Not | <40 Ct |
| | | Detected | |
| Influenza C | +/− | Detected/Not | <40 Ct |
| | | Detected | |
| RP | + | N/A | <38 Ct |

### V. PERFORMANCE VALIDATION

In one aspect, the present disclosure relates to a method for detecting a respiratory illness, comprising: Real-Time Reverse Transcriptase (RT)-PCR assays for the in vitro qualitative detection of nucleic acids, which requires a performance validation.

In some embodiments, performance validation comprises accuracy or trueness. In some embodiments, accuracy is the closeness of the agreement between the results of a single measurement and the true value of the analyte.

In some embodiments, performance validation comprises precision or reproducibility. In some embodiments, precision refers to how well a given measurement can be reproduced when a test is applied repeatedly to multiple aliquots of a single homogeneous sample. In some embodiments, precision refers to how well a given measurement can be reproduced when a test is applied repeatedly is defined as the “closeness of agreement between independent test/measurement results obtained under stipulated conditions”. In some embodiments, the method comprises assessing precision by evaluating reproducibility. In some embodiments, reproducibility is considered to be the extreme measures of precision, with reproducibility (run-to-run imprecision, operator-to-operator imprecision, etc.) being the largest measure of precision and involving results of measurements under changed conditions (different operators, time, etc.).

In some embodiments, performance validation comprises analytical sensitivity. In some embodiments, analytical Sensitivity is defined as the ability of the assay to detect very low concentrations of a given sample. In some embodiments, analytical sensitivity is also referred as Limit of Detection (LoD). In some embodiments, LoD is the lowest actual concentration of the sample that can be consistently detected with acceptable precision.

In some embodiments, performance validation comprises analytical specificity. In some embodiments, analytical specificity refers to the ability of an assay to detect only the intended target and that quantification of the target is not affected by cross-reactivity from related or potentially interfering nucleic acids or specimen related conditions. In some embodiments, the two aspects of analytical specificity of cross-reactivity and interference can be determined by performing interference studies.

In some embodiments, performance validation comprises quality controls. In some embodiments, all controls start at extraction step, which also serve as process control. In some embodiments, controls are taken through all steps of the assay and serve as extraction controls and amplification controls. In some embodiments, the three controls are negative extraction control (EC) from known normal individuals, Positive template control (PTC) from known positive individuals or synthetic control material, and the No Template Control (NTC). In some embodiments, control target values are determined during the validation runs, then control limits based on the analytical variation of the assay as well as the implications of rejecting a test are set.

In some embodiments, the method comprises sample de-identification for use in the method validation protocol. In some embodiments, sample vials used for the validation are obtained from individuals, whereupon all samples are de-identified according to FDA guidance. Alternatively, in some embodiments, if a sample vial is unavailable, previously extracted RNA from a vial may be utilized. In some embodiments, spiking RNA is used as a positive template control. In some embodiments, a set of serial numbers is prepared, with one serial number assigned and labeled per one original vial. In some embodiments, the serial number is recorded in relation to the original vial ID number.

In some embodiments, the method comprises performance specification elements. In some embodiments, performance specification elements comprise: (a) accuracy or trueness; (b) precision or reproducibility comprising: (1) Intra-Assay Reproducibility (Within-Run Precision), (2) Operator-to-Operator Reproducibility, and (3) Inter-Assay Reproducibility (Run-to-Run Precision); (c) analytical sensitivity comprising: (1) In silico Inclusivity, (2) Limit of Detection (LoD); (d) analytical Specificity that is In silico Cross-Reactivity; and (e) Quality Control and Quality Metrics.

In some embodiments, the method comprises a validation procedure. In some embodiments, the validation procedure comprises accuracy or trueness. In some embodiments, in the absence of known positive samples for testing, the FDA recommends that laboratories confirm performance of their assay with a series of contrived clinical specimens by testing a minimum of 30 contrived reactive(positive) specimens and 30 non-reactive (negative) specimens. In some embodiments, contrived reactive specimens can be created by spiking RNA or inactivated virus into clinical specimens: (1) Sample 1˜30: Negative sample; (2) Sample 31˜60: Positive sample. In some embodiments, the accuracy is assessed by measure of agreement of the samples that have the same calls. The % accuracy is assessed by the following formula, with the acceptance critera being ≥95%:

% accuracy=(#samples called accurately)/(total #of samples)×100%

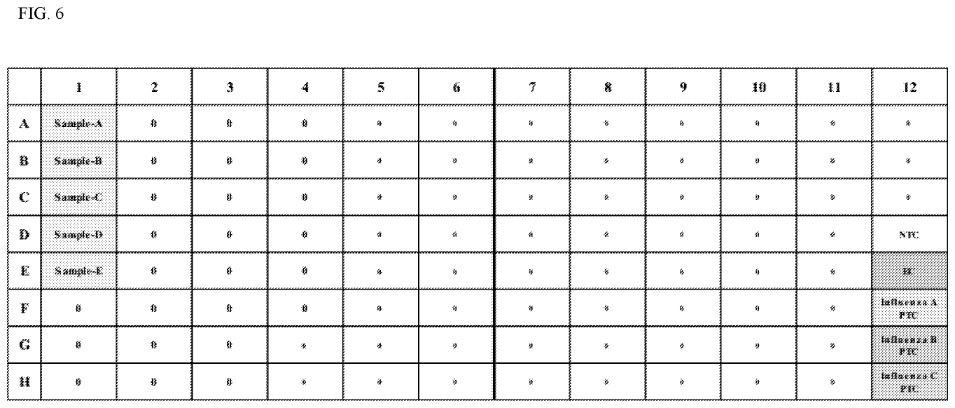

In some embodiments, the validation procedure comprises precision or reproducibility. In some embodiments, precision is related to within and between run controls to determine ability to return identical results under identical (precision) or changed (reproducibility) conditions (FIG. 6).

In some embodiments, the following runs are performed to verify precision or reproducibility: (a) intra-assay reproducibility (Within-Run Precision) comprising determining by testing five known positive (spiking RNA) samples in one run; (b) Operator-to-Operator and Inter-Assay Reproducibility (Run-to-Run Precision) comprising determining by testing five positive (spiking RNA) samples by three different operators in five different runs; (c) data analysis, wherein the data are expressed as two-level categorical variables (i.e., ‘concordant’ or ‘discordant’) and expressed as a percent ‘concordance’, wherein at the conclusion of this experiment, the concordance from each reproducibility category is calculated and the negative or positive calls evaluated with the variable conditions listed, and wherein percent concordance is determined by the following formula, with the acceptance criteria being ≥95% concordance:

% Concordance=(#of samples called correctly/Total #of samples)×100%

In some embodiments, the validation procedure comprises Analytical Sensitivity. In some embodiments, the analytical sensitivity comprises In silico Inclusivity. In some embodiments, results are documented of an inclusivity study that demonstrates the strains of SARS-CoV-2 that can be detected by the proposed molecular assay. In some embodiments, it is acceptable to conduct an in silico analysis of published SARS-CoV-2 sequences using the assay's primers and probes. In some embodiments, the FDA anticipates that 100% of published SARS-CoV-2 sequences are detectable with the selected primers and probes.

In some embodiments, the analytical sensitivity comprises Limit of Detection (LoD). In some embodiments, analytical Sensitivity refers to the true positives in agreement of results when compared to a reference method. In some embodiments, LoD is determined by the following calculation:

Analytical Sensitivity=TP/(TP+FN)×100%

where TP represents the number of True Positives and FN represents the number of False Negatives and the acceptance Criteria≥95%.

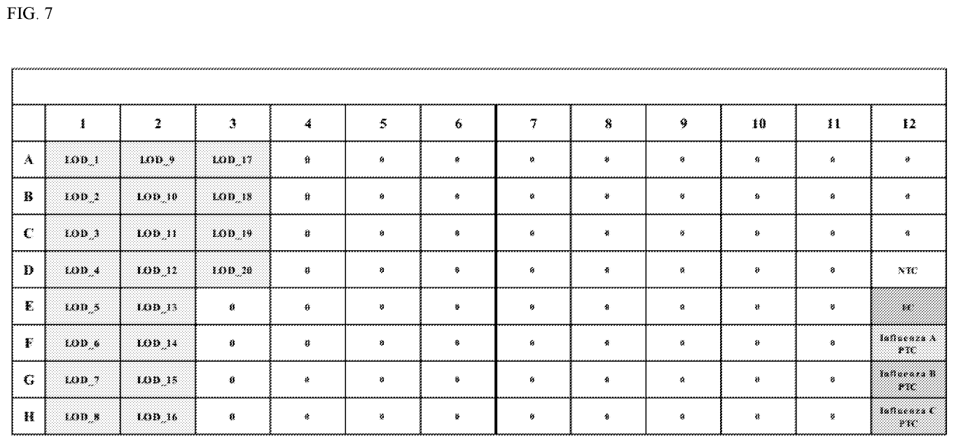

In some embodiments, the Limit of Detection (LoD) is determined empirically by testing serial dilutions of three replicates per concentration, and then the final concentration confirmed with 20 replicates. The LoD is defined as the lowest concentration at which 19/20 replicates are positive. Table 13 shows an analytical sensitivity sample list and FIG. 7 shows an analytical sensitivity plate layout.

TABLE 13
Analytical sensitivity sample list
Three positive samples (Spiking RNA) List
Stock
Stock
Stock
1:10 Stock Dilution
1:10 Stock Dilution
1:10 Stock Dilution
1:100 Stock Dilution
1:100 Stock Dilution
1:100 Stock Dilution
1:1000 Stock Dilution
1:1000 Stock Dilution
1:1000 Stock Dilution
1:10000 Stock Dilution
1:10000 Stock Dilution
1:10000 Stock Dilution
1:100000 Stock Dilution
1:100000 Stock Dilution
1:100000 Stock Dilution

In some embodiments, the validation procedure comprises analytical specificity. In some embodiments, the analytical specificity comprises in silico Cross-Reactivity. In some embodiments, at a minimum, an in silico analysis of the assay primer and probes compared to common respiratory flora and other viral pathogens is sufficient for initial clinical use. In some embodiments, FDA defines in silico cross-reactivity as greater than 80% homology between one of the primers/probes and any sequence present in the targeted microorganism. In some embodiments, recognized laboratory procedures are followed in the context of the sample types intended for testing for any additional cross-reactivity testing. Table 14 shows a recommended List of Organisms to be Analyzed in silico or by Direct Testing.

TABLE 14
Recommended List of organisms to be analyzed in silico or by direct testing

| Other high priority pathogens | High priority organisms likely in |
|---|---|
| from the same genetic family | circulating areas |
| Human coronavirus 229E | Adenovirus (e.g. C1 Ad. 71) |
| Human coronavirus OC43 | Human Metapneumovirus (hMPV) |
| Human coronavirus HKU1 | Parainfluenza virus 1-4 |
| Human coronavirus NL63 | Influenza A & B |
| SARS-coronavirus | Enterovirus (e.g. EV68) |
| MERS-coronavirus | Respiratory syncytial virus |
| | Rhinovirus |
| | Chlamydia pneumoniae |
| | Haemophilus influenzae |
| | Legionella pneumophila |
| | Mycobacterium tuberculosis |
| | Streptococcus pneumoniae |
| | Streptococcus pyogenes |
| | Bordetella pertussis |
| | Mycoplasma pneumoniae |
| | Pneumocystis jiroveci (PJP) |
| | Pooled human nasal wash-to represent |
| | diverse microbial flora in the human |
| | respiratory tract |
| | Candida albicans |
| | Pseudomonas aeruginosa |
| | Staphylococcus epidermis |
| | Staphylococcus Streptococcus salivarius |

In some embodiments, the validation procedure comprises establishing Quality Control and Quality Metrics. In some embodiments, quality metrics for Evaluating a Run comprise: (1) Verifying the NTC, EC, and PTC controls and amplifying as expected; (2) Verifying RP samples are positive at or before 38 cycles for all patient samples and EC; and (3) Verifying Ct is less than 40 cycles for PTC targets.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Assessment of Methods for Detecting a Panel of Viruses Causing Respiratory Illness

### Performance Characteristics Evalution Procedure

The following performance characteristics will be evaluated in accordance the Validation Protocol: (1) Accuracy (Trueness); (2) Precision (Reproducibility), including: (a) Intra-Assay Reproducibility (Within-Run Precision), (b) Operator-to-Operator Reproducibility, (c) Inter-Assay Reproducibility (Run-to-Run Precision); (3) Analytical Sensitivity, including (a) Limit of Detection (LoD); and (4) Quality Control.

### Performance Characteristics Summary: Accuracy (Trueness)

Accuracy Acceptance Criteria. The Accuracy Acceptance Criteria will be as follows. Percent Accuracy will be assessed by the following formula, with the acceptance criteria being ≥95%:

% accuracy=(#samples called accurately)/(total #of samples)×100%

Accuracy Data Summary. There are total 30 negative samples and 30 positive samples for each of the Influenza A, B and C combined in the same well. The sample numbers are the following: (1) 30 Accuracy Negative: Sample 1-Sample 30; and (2) 30 Accuracy Positive for Influenza A, B and C: Sample 31-Sample 60. Sample results were compared to the corresponding theoretical results and the data are presented in Tables 15 and 16.

TABLE 15
Accuracy Negative Sample Result Comparison

| | Influenza A B C (RT)- | | | | | |
|---|---|---|---|---|---|---|
| | PCR Panel (C_{T}) | | | | | |
| | Influ- | Influ- | Influ- | | | |
| | enza | enza | enza | | Actual | Theo- |
| Sample | A | B | C | | Result | retical |
| Name | Ct | Ct | Ct | RP | Actual | Result |

Sample 1~Sample 30, Negative

| | | | | | | |
|---|---|---|---|---|---|---|
| Sample | ND* | ND | ND | 31.28 | Negative | Negative |
| 1 | | | | | | |
| Sample | ND | ND | ND | 28.38 | Negative | Negative |
| 2 | | | | | | |
| Sample | ND | ND | ND | 30.30 | Negative | Negative |
| 3 | | | | | | |
| Sample | ND | ND | ND | 28.36 | Negative | Negative |
| 4 | | | | | | |
| Sample | ND | ND | ND | 32.22 | Negative | Negative |
| 5 | | | | | | |
| Sample | ND | ND | ND | 31.58 | Negative | Negative |
| 6 | | | | | | |
| Sample | ND | ND | ND | 28.94 | Negative | Negative |
| 7 | | | | | | |
| Sample | ND | ND | ND | 29.62 | Negative | Negative |
| 8 | | | | | | |
| Sample | ND | ND | ND | 30.96 | Negative | Negative |
| 9 | | | | | | |
| Sample | ND | ND | ND | 29.72 | Negative | Negative |
| 10 | | | | | | |
| Sample | ND | ND | ND | 29.68 | Negative | Negative |
| 11 | | | | | | |
| Sample | ND | ND | ND | 28.27 | Negative | Negative |
| 12 | | | | | | |
| Sample | ND | ND | ND | 27.14 | Negative | Negative |
| 13 | | | | | | |
| Sample | ND | ND | ND | 32.29 | Negative | Negative |
| 14 | | | | | | |
| Sample | ND | ND | ND | 34.94 | Negative | Negative |
| 15 | | | | | | |
| Sample | ND | ND | ND | 33.93 | Negative | Negative |
| 16 | | | | | | |
| Sample | ND | ND | ND | 27.88 | Negative | Negative |
| 17 | | | | | | |
| Sample | ND | ND | ND | 27.19 | Negative | Negative |
| 18 | | | | | | |
| Sample | ND | ND | ND | 30.51 | Negative | Negative |
| 19 | | | | | | |
| Sample | ND | ND | ND | 27.56 | Negative | Negative |
| 20 | | | | | | |
| Sample | ND | ND | ND | 31.81 | Negative | Negative |
| 21 | | | | | | |
| Sample | ND | ND | ND | 31.29 | Negative | Negative |
| 22 | | | | | | |
| Sample | ND | ND | ND | 32.24 | Negative | Negative |
| 23 | | | | | | |
| Sample | ND | ND | ND | 27.69 | Negative | Negative |
| 24 | | | | | | |
| Sample | ND | ND | ND | 28.81 | Negative | Negative |
| 25 | | | | | | |
| Sample | ND | ND | ND | 30.92 | Negative | Negative |
| 26 | | | | | | |
| Sample | ND | ND | ND | 26.57 | Negative | Negative |
| 27 | | | | | | |
| Sample | ND | ND | ND | 26.18 | Negative | Negative |
| 28 | | | | | | |
| Sample | ND | ND | ND | 29.28 | Negative | Negative |
| 29 | | | | | | |
| Sample | ND | ND | ND | 26.73 | Negative | Negative |
| 30 | | | | | | |
| NTC | ND | ND | ND | ND | NTC | NTC |
| EC | ND | ND | ND | 27.33 | Negative | Negative |
| PTC | 15.84 | 25.15 | 22.22 | 31.59 | Positive | Positive |

*ND: Not Detected

TABLE 16
Accuracy Positive Sample Result Comparison

| | Influenza A B C (RT)- | | | | | |
|---|---|---|---|---|---|---|
| | PCR Panel (C_{T}) | | | | | |
| | Influ- | Influ- | Influ- | | | |
| | enza | enza | enza | | Actual | Theo- |
| Sample | A | B | C | | Result | retical |
| Name | Ct | Ct | Ct | RP | Actual | Result |

Sample 31~Sample 60, Positive

| | | | | | | |
|---|---|---|---|---|---|---|
| Sample A- | 24.34 | ND* | ND | 27.69 | Influenza A | Influenza A |
| 31 | | | | | Positive | Positive |
| Sample A- | 24.18 | ND | ND | 31.68 | Influenza A | Influenza A |
| 32 | | | | | Positive | Positive |
| Sample A- | 24.10 | ND | ND | 29.35 | Influenza A | Influenza A |
| 33 | | | | | Positive | Positive |
| Sample A- | 24.24 | ND | ND | 30.32 | Influenza A | Influenza A |
| 34 | | | | | Positive | Positive |
| Sample A- | 23.69 | ND | ND | 31.18 | Influenza A | Influenza A |
| 35 | | | | | Positive | Positive |
| Sample A- | 24.19 | ND | ND | 29.42 | Influenza A | Influenza A |
| 36 | | | | | Positive | Positive |
| Sample A- | 24.24 | ND | ND | 30.96 | Influenza A | Influenza A |
| 37 | | | | | Positive | Positive |
| Sample A- | 23.97 | ND | ND | 31.40 | Influenza A | Influenza A |
| 38 | | | | | Positive | Positive |
| Sample A- | 24.24 | ND | ND | 31.43 | Influenza A | Influenza A |
| 39 | | | | | Positive | Positive |
| Sample A- | 24.27 | ND | ND | 29.93 | Influenza A | Influenza A |
| 40 | | | | | Positive | Positive |
| Sample A- | 24.19 | ND | ND | 31.71 | Influenza A | Influenza A |
| 41 | | | | | Positive | Positive |
| Sample A- | 24.43 | ND | ND | 28.99 | Influenza A | Influenza A |
| 42 | | | | | Positive | Positive |
| Sample A- | 24.46 | ND | ND | 29.00 | Influenza A | Influenza A |
| 43 | | | | | Positive | Positive |
| Sample A- | 24.30 | ND | ND | 29.31 | Influenza A | Influenza A |
| 44 | | | | | Positive | Positive |
| Sample A- | 24.12 | ND | ND | 28.53 | Influenza A | Influenza A |
| 45 | | | | | Positive | Positive |
| Sample A- | 27.55 | ND | ND | 28.65 | Influenza A | Influenza A |
| 46 | | | | | Positive | Positive |
| Sample A- | 27.70 | ND | ND | 29.44 | Influenza A | Influenza A |
| 47 | | | | | Positive | Positive |
| Sample A- | 27.70 | ND | ND | 28.87 | Influenza A | Influenza A |
| 48 | | | | | Positive | Positive |
| Sample A- | 27.88 | ND | ND | 29.17 | Influenza A | Influenza A |
| 49 | | | | | Positive | Positive |
| Sample A- | 27.81 | ND | ND | 29.38 | Influenza A | Influenza A |
| 50 | | | | | Positive | Positive |
| Sample A- | 27.84 | ND | ND | 28.49 | Influenza A | Influenza A |
| 51 | | | | | Positive | Positive |
| Sample A- | 27.73 | ND | ND | 29.09 | Influenza A | Influenza A |
| 52 | | | | | Positive | Positive |
| Sample A- | 27.68 | ND | ND | 29.89 | Influenza A | Influenza A |
| 53 | | | | | Positive | Positive |
| Sample A- | 27.65 | ND | ND | 29.02 | Influenza A | Influenza A |
| 54 | | | | | Positive | Positive |
| Sample A- | 27.73 | ND | ND | 28.45 | Influenza A | Influenza A |
| 55 | | | | | Positive | Positive |
| Sample A- | 27.80 | ND | ND | 26.83 | Influenza A | Influenza A |
| 56 | | | | | Positive | Positive |
| Sample A- | 27.74 | ND | ND | 28.67 | Influenza A | Influenza A |
| 57 | | | | | Positive | Positive |
| Sample A- | 27.88 | ND | ND | 27.79 | Influenza A | Influenza A |
| 58 | | | | | Positive | Positive |
| Sample A- | 27.86 | ND | ND | 27.19 | Influenza A | Influenza A |
| 59 | | | | | Positive | Positive |
| Sample A- | 27.74 | ND | ND | 28.53 | Influenza A | Influenza A |
| 60 | | | | | Positive | Positive |
| Sample B- | ND | 24.07 | ND | 28.69 | Influenza B | Influenza B |
| 31 | | | | | Positive | Positive |
| Sample B- | ND | 24.03 | ND | 28.99 | Influenza B | Influenza B |
| 32 | | | | | Positive | Positive |
| Sample B- | ND | 24.12 | ND | 27.82 | Influenza B | Influenza B |
| 33 | | | | | Positive | Positive |
| Sample B- | ND | 24.05 | ND | 27.04 | Influenza B | Influenza B |
| 34 | | | | | Positive | Positive |
| Sample B- | ND | 23.96 | ND | 28.70 | Influenza B | Influenza B |
| 35 | | | | | Positive | Positive |
| Sample B- | ND | 23.84 | ND | 30.03 | Influenza B | Influenza B |
| 36 | | | | | Positive | Positive |
| Sample B- | ND | 23.92 | ND | 25.43 | Influenza B | Influenza B |
| 37 | | | | | Positive | Positive |
| Sample B- | ND | 23.91 | ND | 26.43 | Influenza B | Influenza B |
| 38 | | | | | Positive | Positive |
| Sample B- | ND | 23.96 | ND | 28.44 | Influenza B | Influenza B |
| 39 | | | | | Positive | Positive |
| Sample B- | ND | 24.01 | ND | 28.37 | Influenza B | Influenza B |
| 40 | | | | | Positive | Positive |
| Sample B- | ND | 24.21 | ND | 27.79 | Influenza B | Influenza B |
| 41 | | | | | Positive | Positive |
| Sample B- | ND | 23.87 | ND | 31.56 | Influenza B | Influenza B |
| 42 | | | | | Positive | Positive |
| Sample B- | ND | 23.97 | ND | 29.11 | Influenza B | Influenza B |
| 43 | | | | | Positive | Positive |
| Sample B- | ND | 23.95 | ND | 30.16 | Influenza B | Influenza B |
| 44 | | | | | Positive | Positive |
| Sample B- | ND | 23.90 | ND | 30.07 | Influenza B | Influenza B |
| 45 | | | | | Positive | Positive |
| Sample B- | ND | 27.47 | ND | 28.98 | Influenza B | Influenza B |
| 46 | | | | | Positive | Positive |
| Sample B- | ND | 27.55 | ND | 30.81 | Influenza B | Influenza B |
| 47 | | | | | Positive | Positive |
| Sample B- | ND | 27.57 | ND | 31.33 | Influenza B | Influenza B |
| 48 | | | | | Positive | Positive |
| Sample B- | ND | 27.69 | ND | 31.31 | Influenza B | Influenza B |
| 49 | | | | | Positive | Positive |
| Sample B- | ND | 27.55 | ND | 29.69 | Influenza B | Influenza B |
| 50 | | | | | Positive | Positive |
| Sample B- | ND | 27.49 | ND | 31.54 | Influenza B | Influenza B |
| 51 | | | | | Positive | Positive |
| Sample B- | ND | 27.56 | ND | 28.92 | Influenza B | Influenza B |
| 52 | | | | | Positive | Positive |
| Sample B- | ND | 27.68 | ND | 28.93 | Influenza B | Influenza B |
| 53 | | | | | Positive | Positive |
| Sample B- | ND | 27.50 | ND | 29.18 | Influenza B | Influenza B |
| 54 | | | | | Positive | Positive |
| Sample B- | ND | 27.39 | ND | 28.47 | Influenza B | Influenza B |
| 55 | | | | | Positive | Positive |
| Sample B- | ND | 27.56 | ND | 28.52 | Influenza B | Influenza B |
| 56 | | | | | Positive | Positive |
| Sample B- | ND | 27.70 | ND | 31.38 | Influenza B | Influenza B |
| 57 | | | | | Positive | Positive |
| Sample B- | ND | 27.60 | ND | 29.73 | Influenza B | Influenza B |
| 58 | | | | | Positive | Positive |
| Sample B- | ND | 27.46 | ND | 31.58 | Influenza B | Influenza B |
| 59 | | | | | Positive | Positive |
| Sample B- | ND | 27.63 | ND | 28.68 | Influenza B | Influenza B |
| 60 | | | | | Positive | Positive |
| Sample C- | ND | ND | 27.02 | 28.95 | Influenza C | Influenza C |
| 31 | | | | | Positive | Positive |
| Sample C- | ND | ND | 26.71 | 29.05 | Influenza C | Influenza C |
| 32 | | | | | Positive | Positive |
| Sample C- | ND | ND | 26.60 | 28.16 | Influenza C | Influenza C |
| 33 | | | | | Positive | Positive |
| Sample C- | ND | ND | 26.22 | 28.55 | Influenza C | Influenza C |
| 34 | | | | | Positive | Positive |
| Sample C- | ND | ND | 26.42 | 28.57 | Influenza C | Influenza C |
| 35 | | | | | Positive | Positive |
| Sample C- | ND | ND | 26.03 | 26.85 | Influenza C | Influenza C |
| 36 | | | | | Positive | Positive |
| Sample C- | ND | ND | 26.03 | 28.60 | Influenza C | Influenza C |
| 37 | | | | | Positive | Positive |
| Sample C- | ND | ND | 25.96 | 27.40 | Influenza C | Influenza C |
| 38 | | | | | Positive | Positive |
| Sample C- | ND | ND | 25.97 | 27.22 | Influenza C | Influenza C |
| 39 | | | | | Positive | Positive |
| Sample C- | ND | ND | 25.96 | 28.44 | Influenza C | Influenza C |
| 40 | | | | | Positive | Positive |
| Sample C- | ND | ND | 26.00 | 28.82 | Influenza C | Influenza C |
| 41 | | | | | Positive | Positive |
| Sample C- | ND | ND | 25.88 | 29.00 | Influenza C | Influenza C |
| 42 | | | | | Positive | Positive |
| Sample C- | ND | ND | 26.32 | 27.69 | Influenza C | Influenza C |
| 43 | | | | | Positive | Positive |
| Sample C- | ND | ND | 25.54 | 31.54 | Influenza C | Influenza C |
| 44 | | | | | Positive | Positive |
| Sample C- | ND | ND | 25.77 | 29.11 | Influenza C | Influenza C |
| 45 | | | | | Positive | Positive |
| Sample C- | ND | ND | 29.59 | 30.14 | Influenza C | Influenza C |
| 46 | | | | | Positive | Positive |
| Sample C- | ND | ND | 29.65 | 30.19 | Influenza C | Influenza C |
| 47 | | | | | Positive | Positive |
| Sample C- | ND | ND | 29.58 | 29.22 | Influenza C | Influenza C |
| 48 | | | | | Positive | Positive |
| Sample C- | ND | ND | 29.54 | 30.85 | Influenza C | Influenza C |
| 49 | | | | | Positive | Positive |
| Sample C- | ND | ND | 29.61 | 31.69 | Influenza C | Influenza C |
| 50 | | | | | Positive | Positive |
| Sample C- | ND | ND | 29.71 | 28.34 | Influenza C | Influenza C |
| 51 | | | | | Positive | Positive |
| Sample C- | ND | ND | 28.98 | 27.40 | Influenza C | Influenza C |
| 52 | | | | | Positive | Positive |
| Sample C- | ND | ND | 29.11 | 28.90 | Influenza C | Influenza C |
| 53 | | | | | Positive | Positive |
| Sample C- | ND | ND | 29.36 | 28.21 | Influenza C | Influenza C |
| 54 | | | | | Positive | Positive |
| Sample C- | ND | ND | 29.25 | 27.88 | Influenza C | Influenza C |
| 55 | | | | | Positive | Positive |
| Sample C- | ND | ND | 29.20 | 28.99 | Influenza C | Influenza C |
| 56 | | | | | Positive | Positive |
| Sample C- | ND | ND | 29.43 | 29.31 | Influenza C | Influenza C |
| 57 | | | | | Positive | Positive |
| Sample C- | ND | ND | 29.48 | 29.00 | Influenza C | Influenza C |
| 58 | | | | | Positive | Positive |
| Sample C- | ND | ND | 29.84 | 29.80 | Influenza C | Influenza C |
| 59 | | | | | Positive | Positive |
| Sample C- | ND | ND | 29.65 | 29.13 | Influenza C | Influenza C |
| 60 | | | | | Positive | Positive |
| NTC | ND | ND | ND | ND | NTC | NTC |
| EC | ND | ND | ND | 27.30 | Negative | Negative |
| PTC | 17.45 | 18.37 | 19.14 | 28.20 | Positive | Positive |

*ND: Not Detected

TABLE 17
SARC-CoV2, SARS-like Coronavirus and RSV (RT)-PCR Panel (CT)

| | SARC-CoV2, | | | | | |
|---|---|---|---|---|---|---|
| | SARS-like Coronavirus and | | | | | |
| | RSV (RT)-PCR Panel (CT) | | | | | |
| | | SARS- | | | | |
| | | like | | | | |
| | SARC- | Corona- | | | | Theo- |
| Sample | CoV2 | virus | RSV | | Actual | retical |
| Name | Ct | Ct | Ct | RP | Result | Result |

Sample 31~Sample 60, Positive

| | | | | | | |
|---|---|---|---|---|---|---|
| Sample R- | ND | ND | 35.96 | 29.94 | RSV Positive | RSV Positive |
| 31 | | | | | | |
| Sample R- | ND | ND | 35.96 | 29.92 | RSV Positive | RSV Positive |
| 32 | | | | | | |
| Sample R- | ND | ND | 35.66 | 29.9 | RSV Positive | RSV Positive |
| 33 | | | | | | |
| Sample R- | ND | ND | 35.61 | 29.95 | RSV Positive | RSV Positive |
| 34 | | | | | | |
| Sample R- | ND | ND | 35.82 | 29.9 | RSV Positive | RSV Positive |
| 35 | | | | | | |
| Sample R- | ND | ND | 36.18 | 29.99 | RSV Positive | RSV Positive |
| 36 | | | | | | |
| Sample R- | ND | ND | 35.82 | 29.98 | RSV Positive | RSV Positive |
| 37 | | | | | | |
| Sample R- | ND | ND | 35.53 | 29.81 | RSV Positive | RSV Positive |
| 38 | | | | | | |
| Sample R- | ND | ND | 36.36 | 29.81 | RSV Positive | RSV Positive |
| 39 | | | | | | |
| Sample R- | ND | ND | 36.13 | 30.02 | RSV Positive | RSV Positive |
| 40 | | | | | | |
| Sample R- | ND | ND | 35.91 | 29.86 | RSV Positive | RSV Positive |
| 41 | | | | | | |
| Sample R- | ND | ND | 35.87 | 29.91 | RSV Positive | RSV Positive |
| 42 | | | | | | |
| Sample R- | ND | ND | 35.73 | 29.9 | RSV Positive | RSV Positive |
| 43 | | | | | | |
| Sample R- | ND | ND | 35.86 | 29.96 | RSV Positive | RSV Positive |
| 44 | | | | | | |
| Sample R- | ND | ND | 35.81 | 29.92 | RSV Positive | RSV Positive |
| 45 | | | | | | |
| Sample R- | ND | ND | 34.47 | 29.69 | RSV Positive | RSV Positive |
| 46 | | | | | | |
| Sample R- | ND | ND | 34.37 | 29.7 | RSV Positive | RSV Positive |
| 47 | | | | | | |
| Sample R- | ND | ND | 34.23 | 29.78 | RSV Positive | RSV Positive |
| 48 | | | | | | |
| Sample R- | ND | ND | 34.31 | 29.81 | RSV Positive | RSV Positive |
| 49 | | | | | | |
| Sample R- | ND | ND | 34.29 | 29.81 | RSV Positive | RSV Positive |
| 50 | | | | | | |
| Sample R- | ND | ND | 34.07 | 29.73 | RSV Positive | RSV Positive |
| 51 | | | | | | |
| Sample R- | ND | ND | 34.15 | 29.82 | RSV Positive | RSV Positive |
| 52 | | | | | | |
| Sample R- | ND | ND | 34.48 | 29.74 | RSV Positive | RSV Positive |
| 53 | | | | | | |
| Sample R- | ND | ND | 34.41 | 29.75 | RSV Positive | RSV Positive |
| 54 | | | | | | |
| Sample R- | ND | ND | 34.1 | 29.8 | RSV Positive | RSV Positive |
| 55 | | | | | | |
| Sample R- | ND | ND | 34.14 | 29.79 | RSV Positive | RSV Positive |
| 56 | | | | | | |
| Sample R- | ND | ND | 34.32 | 29.87 | RSV Positive | RSV Positive |
| 57 | | | | | | |
| Sample R- | ND | ND | 34.5 | 29.76 | RSV Positive | RSV Positive |
| 58 | | | | | | |
| Sample R- | ND | ND | 34.33 | 29.77 | RSV Positive | RSV Positive |
| 59 | | | | | | |
| Sample R- | ND | ND | 34.08 | 29.82 | RSV Positive | RSV Positive |
| 60 | | | | | | |
| NTC | ND | ND | ND | ND | NTC | NTC |
| EC | ND | ND | ND | 29.30 | Negative | Negative |
| SARS | 31.50 | 34.48 | ND | 34.28 | Positive | Positive |
| PTC | | | | | | |
| RSV PTC | ND | ND | 16.61 | 26.40 | Positive | Positive |

Accuracy Result Summary. Accuracy was defined as follows:

% Accuracy=(60 samples called accurately)/(60 total samples)=100%

59 samples' experimental results matched their theoretical results. One sample, Sample56, failed to detect the T1 and T2 target and called the sample negative. The data demonstrated 100% accuracy. Target performance goal is ≥95% accuracy. Therefore, Accuracy target performance was achieved. It is concluded that the Accuracy performance characteristic is validated and have met acceptance criteria for this testing system.

The data demonstrated 100% accuracy. Target performance goal is ≥95% accuracy. Therefore, Accuracy target performance was achieved. It is concluded that the Accuracy performance characteristic is validated and have met acceptance criteria for this testing system.

### Performance Characteristics Summary: Precision (Reproducibility)

Precision Acceptance Criteria. Percent Concordance will be assessed by the following formula, with the acceptance criteria being ≥95% concordance of sample results for all samples tested:

% Concordance=(#of samples called correctly/Total #of samples)×100%

TABLE 18
Operator-to-Operator and Inter-Assay Reproducibility Result Data
Operator-to-Operator and Inter-Assay Reproducibility Result Data

| | | | C_{T} | | | | | |
|---|---|---|---|---|---|---|---|---|
| | | Sample | Influenza | Influenza | Influenza | | Actual | Expected |
| Run | Operator | Name | A | B | C | RP | Result | Result |
| 1 | 1 | Sample A | ND* | ND | ND | 29.45 | Negative | Negative |
| | | Sample B | ND | ND | ND | 28.82 | Negative | Negative |
| | | Sample C | ND | ND | ND | 29.42 | Negative | Negative |
| | | Sample D | ND | ND | ND | 27.74 | Negative | Negative |
| | | Sample E | ND | ND | ND | 26.28 | Negative | Negative |
| | | NTC | ND | ND | ND | ND | Negative | Negative |
| | | EC | ND | ND | ND | 26.64 | Negative | Negative |
| | | PTC | 24.62 | 27.65 | 28.48 | 29.18 | Positive | Positive |
| 2 | 2 | Sample A | ND | ND | ND | 29.06 | Negative | Negative |
| | | Sample B | ND | ND | ND | 31.67 | Negative | Negative |
| | | Sample C | ND | ND | ND | 29.50 | Negative | Negative |
| | | Sample D | ND | ND | ND | 29.02 | Negative | Negative |
| | | Sample E | ND | ND | ND | 28.98 | Negative | Negative |
| | | NTC | ND | ND | ND | ND | Negative | Negative |
| | | EC | ND | ND | ND | 27.98 | Negative | Negative |
| | | PTC | 24.93 | 24.28 | 25.88 | 29.16 | Positive | Positive |
| 3 | 3 | Sample A | ND | ND | ND | 29.61 | Negative | Negative |
| | | Sample B | ND | ND | ND | 28.87 | Negative | Negative |
| | | Sample C | ND | ND | ND | 29.46 | Negative | Negative |
| | | Sample D | ND | ND | ND | 27.79 | Negative | Negative |
| | | Sample E | ND | ND | ND | 26.18 | Negative | Negative |
| | | NTC | ND | ND | ND | ND | Negative | Negative |
| | | EC | ND | ND | ND | 26.80 | Negative | Negative |
| | | PTC | 24.42 | 28.13 | 29.63 | 28.95 | Positive | Positive |
| 4 | 4 | Sample A | ND | ND | ND | 29.65 | Negative | Negative |
| | | Sample B | ND | ND | ND | 29.07 | Negative | Negative |
| | | Sample C | ND | ND | ND | 29.68 | Negative | Negative |
| | | Sample D | ND | ND | ND | 27.91 | Negative | Negative |
| | | Sample E | ND | ND | ND | 26.32 | Negative | Negative |
| | | NTC | ND | ND | ND | ND | Negative | Negative |
| | | EC | ND | ND | ND | 26.95 | Negative | Negative |
| | | PTC | 25.93 | 28.01 | 27.99 | 28.99 | Positive | Positive |
| 5 | 5 | Sample A | ND | ND | ND | 25.95 | Negative | Negative |
| | | Sample B | ND | ND | ND | 29.14 | Negative | Negative |
| | | Sample C | ND | ND | ND | 25.76 | Negative | Negative |
| | | Sample D | ND | ND | ND | 28.42 | Negative | Negative |
| | | Sample E | ND | ND | ND | 28.33 | Negative | Negative |
| | | NTC | ND | ND | ND | ND | Negative | Negative |
| | | EC | ND | ND | ND | 28.58 | Negative | Negative |
| | | PTC | 29.14 | 28.41 | 30.07 | 29.53 | Positive | Positive |

*ND: Not Detected

TABLE 19
Operator-to-Operator and Inter-Assay Reproducibility Result Data
Operator-to-Operator and Inter-Assay Reproducibility Result Data

| | | | C_{T} | | | | | |
|---|---|---|---|---|---|---|---|---|
| | | Sample | SARS- | SARS-like | | | Actual | Expected |
| Run | Operator | Name | CoV2 | Coronavirus | RSV | RP | Result | Result |
| 1 | 1 | Sample | ND | ND | ND | 32.87 | Negative | Negative |
| | | A | | | | | | |
| | | Sample | ND | ND | ND | 32.97 | Negative | Negative |
| | | B | | | | | | |
| | | Sample | ND | ND | ND | 32.42 | Negative | Negative |
| | | C | | | | | | |
| | | Sample | ND | ND | ND | 32.38 | Negative | Negative |
| | | D | | | | | | |
| | | Sample | ND | ND | ND | 33.77 | Negative | Negative |
| | | E | | | | | | |
| | | NTC | ND | ND | ND | ND | Negative | Negative |
| | | EC | ND | ND | ND | 29.25 | Negative | Negative |
| | | SARS | 31.42 | 33.75 | ND | 32.78 | Positive | Positive |
| | | PTC | | | | | | |
| | | RSV | ND | ND | 35.2 | 27.94 | Positive | Positive |
| | | PTC | | | | | | |
| 2 | 2 | Sample | ND | ND | ND | 27.69 | Negative | Negative |
| | | A | | | | | | |
| | | Sample | ND | ND | ND | 28.28 | Negative | Negative |
| | | B | | | | | | |
| | | Sample | ND | ND | ND | 28.37 | Negative | Negative |
| | | C | | | | | | |
| | | Sample | ND | ND | ND | 27.44 | Negative | Negative |
| | | D | | | | | | |
| | | Sample | ND | ND | ND | 31.96 | Negative | Negative |
| | | E | | | | | | |
| | | NTC | ND | ND | ND | ND | Negative | Negative |
| | | EC | ND | ND | ND | 26.97 | Negative | Negative |
| | | SARS | 29.55 | 33.75 | ND | 31.49 | Positive | Positive |
| | | PTC | | | | | | |
| | | RSV | ND | ND | 38.22 | 27.68 | Positive | Positive |
| | | PTC | | | | | | |
| 3 | 3 | Sample | ND | ND | ND | 29.24 | Negative | Negative |
| | | A | | | | | | |
| | | Sample | ND | ND | ND | 28.03 | Negative | Negative |
| | | B | | | | | | |
| | | Sample | ND | ND | ND | 25.94 | Negative | Negative |
| | | C | | | | | | |
| | | Sample | ND | ND | ND | 27.12 | Negative | Negative |
| | | D | | | | | | |
| | | Sample | ND | ND | ND | 25.4 | Negative | Negative |
| | | E | | | | | | |
| | | NTC | ND | ND | ND | ND | Negative | Negative |
| | | EC | ND | ND | ND | 29.97 | Negative | Negative |
| | | SARS | 28.91 | 33.03 | ND | 30.98 | Positive | Positive |
| | | PTC | | | | | | |
| | | RSV | ND | ND | 16.59 | 24.38 | Positive | Positive |
| | | PTC | | | | | | |
| 4 | 4 | Sample | ND | ND | ND | 27.7 | Negative | Negative |
| | | A | | | | | | |
| | | Sample | ND | ND | ND | 38.73 | Negative | Negative |
| | | B | | | | | | |
| | | Sample | ND | ND | ND | 30.08 | Negative | Negative |
| | | C | | | | | | |
| | | Sample | ND | ND | ND | 28.09 | Negative | Negative |
| | | D | | | | | | |
| | | Sample | ND | ND | ND | 25.23 | Negative | Negative |
| | | E | | | | | | |
| | | NTC | ND | ND | ND | ND | Negative | Negative |
| | | EC | ND | ND | ND | 27.55 | Negative | Negative |
| | | SARS | 29.16 | 33.79 | ND | 30.97 | Positive | Positive |
| | | PTC | | | | | | |
| | | RSV | ND | ND | 17.14 | 23.69 | Positive | Positive |
| | | PTC | | | | | | |
| 5 | 5 | Sample | ND | ND | ND | 28.32 | Negative | Negative |
| | | A | | | | | | |
| | | Sample | ND | ND | ND | 28.79 | Negative | Negative |
| | | B | | | | | | |
| | | Sample | ND | ND | ND | 28.1 | Negative | Negative |
| | | C | | | | | | |
| | | Sample | ND | ND | ND | 28.98 | Negative | Negative |
| | | D | | | | | | |
| | | Sample | ND | ND | ND | 29.87 | Negative | Negative |
| | | E | | | | | | |
| | | NTC | ND | ND | ND | ND | Negative | Negative |
| | | EC | ND | ND | ND | 27.90 | Negative | Negative |
| | | SARS | 31.57 | 33.59 | ND | 30.99 | Positive | Positive |
| | | PTC | | | | | | |
| | | RSV | ND | ND | 17.60 | 24.21 | Positive | Positive |
| | | PTC | | | | | | |

*ND: Not Detected

Precision Result Summary. Percent concordance for each precision sample and total concordance is determined to be 100% based on the following formula:

% Concordance=(#of samples called correctly/Total #of samples)×100%

Intra-Assay (Within-Run) Precision:

% Concordance=(5 samples called correctly/5 samples total)×100%

### % Concordance=100%

Operator-to-Operator and Run-to-Run Precision:

% Concordance=(15 samples called correctly/15 samples total)×100%

### % Concordance=100%

Total Precision:

% Concordance=(25 samples called correctly/25 samples total)×100%

### % Concordance=100%

Precision result is 100% concordance (within-run, operator-to-operator). This study was carried out by testing five known positive samples in one run and by testing five (5) positive samples by three (3) different operators in three (3) different runs.

The data demonstrated 100% concordance. Target performance is ≥95% concordance of sample results for all samples tested. Therefore, Precision target performance was achieved. It is concluded that the Precision performance characteristic is validated and have met acceptance criteria for this testing system.

### Performance Characteristics Summary: Analytical Sensitivity

Note: Since the SARS-CoV2 and SARS-like Coronavirus LoD were established by EUA200006, no further LoD is needed for these two targets.

Analytical Sensitivity includes Limit of Detection and in silico Inclusivity Studies. Their acceptance criteria is defined as the following. Analytical Sensitivity is determined by the following calculation:

Analytical Sensitivity=TP/(TP+FN)×100%

where TP represents the number of True Positives and FN represents the number of False Negatives, with the Acceptance Criteria being ≥95%

The Limit of Detection (LoD) is defined as the lowest concentration at which 19/20 replicates are positive.

Limit of Detection Data Summary. The LoD of the AvellinoCoV2 Respiratory Panel was determined using quantified whole viral genomic RNA. Genomic RNA from the Influenza A, B and C were received from BEI Resources.
Influenza A: Catalog #, Lot #, original concentration of zzzz genome equivalents/mL (zzzzz genome equivalents/uL)
Influenza B: Catalog #, Lot #, original concentration of zzzz genome equivalents/mL (zzzzz genome equivalents/uL)
Influenza C: Catalog #, Lot #, original concentration of zzzz genome equivalents/mL (zzzzz genome equivalents/uL)
RSV: Catalog #, Lot #, original concentration of zzzz genome equivalents/mL (zzzzz genome equivalents/uL)

LoD was determined by testing in triplicate serial dilutions of the whole viral genomic RNA spiked into pooled samples. The LoD was confirmed by testing 20 replicates. The limit of detection for the test is now 1 copies/uL.

TABLE 20
LoD Sample Data Summary

| Gene copies/ | Influenza | Influenza | Influenza | |
|---|---|---|---|---|
| reaction | A | B | C | RP |
| (copies/uL) | Ct | Ct | Ct | Ct |
| 2000 | 17.00 | 19.91 | 29.05 | 28.78 |
| 2000 | 16.83 | 19.34 | 29.70 | 29.02 |
| 2000 | 16.82 | 19.26 | 29.91 | 28.86 |
| 200 | 23.38 | 23.54 | 30.85 | 29.44 |
| 200 | 23.63 | 23.93 | 30.77 | 29.54 |
| 200 | 23.58 | 23.82 | 31.62 | 29.45 |
| 100 | 24.69 | 25.46 | 31.09 | 29.18 |
| 100 | 24.36 | 24.89 | 32.74 | 29.65 |
| 100 | 24.29 | 25.13 | 31.24 | 29.47 |
| 50 | 25.47 | 25.98 | 32.30 | 29.29 |
| 50 | 25.77 | 26.11 | 33.55 | 29.13 |
| 50 | 25.85 | 25.98 | 32.97 | 28.97 |
| 20 | 27.22 | 27.27 | 33.86 | 29.22 |
| 20 | 27.25 | 27.40 | 34.27 | 29.37 |
| 20 | 27.17 | 27.51 | 33.96 | 29.25 |
| 10 | 28.66 | 29.15 | 34.78 | 30.07 |
| 10 | 28.34 | 28.72 | 33.82 | 29.64 |
| 10 | 28.08 | 28.60 | 34.21 | 29.67 |
| 5 | 29.31 | 29.57 | 36.59 | 29.88 |
| 5 | 29.41 | 29.56 | 37.42 | 29.63 |
| 5 | 29.41 | 29.58 | 37.28 | 29.58 |
| 1 | 35.03 | 34.45 | 36.94 | 29.48 |
| 1 | 35.90 | 34.79 | 37.36 | 29.61 |
| 1 | 35.21 | 34.26 | 36.51 | 29.85 |
| 0.5 | 37.53 | 37.22 | ND | 30.60 |
| 0.5 | 37.30 | 37.22 | ND | 30.95 |
| 0.5 | ND* | ND | ND | 30.82 |
| 0.05 | ND | ND | ND | 28.64 |
| 0.05 | ND | ND | ND | 28.88 |
| 0.05 | ND | ND | ND | 29.06 |
| NTC | ND | ND | ND | ND |
| EC | ND | ND | ND | 26.84 |
| PTC | 22.56 | 24.03 | 25.08 | 26.67 |

*ND: Not Detected

TABLE 21
LoD Data Summary

| Gene copies/ | SARS- | SARS-like | | |
|---|---|---|---|---|
| reaction | CoV2 | Coronavirus | RSV | RP |
| (copies/uL) | Ct | Ct | Ct | Ct |
| 5000 | ND | ND | 21.1 | 28.95 |
| 5000 | ND | ND | 20.95 | 28.95 |
| 5000 | ND | ND | 20.73 | 29.02 |
| 500 | ND | ND | 26.81 | 28.55 |
| 500 | ND | ND | 26.35 | 28.8 |
| 500 | ND | ND | 26.17 | 29.02 |
| 50 | ND | ND | 29.99 | 28.78 |
| 50 | ND | ND | 29.56 | 28.78 |
| 50 | ND | ND | 29.37 | 28.98 |
| 10 | ND | ND | 31.91 | 28.68 |
| 10 | ND | ND | 31.53 | 28.71 |
| 10 | ND | ND | 31.58 | 28.89 |
| 5 | ND | ND | 32.67 | 28.69 |
| 5 | ND | ND | 32.46 | 28.86 |
| 5 | ND | ND | 32.49 | 28.91 |
| 1 | ND | ND | 34.56 | 28.66 |
| 1 | ND | ND | 34.55 | 28.77 |
| 1 | ND | ND | 34.75 | 28.75 |
| 0.5 | ND | ND | ND | 28.79 |
| 0.5 | ND | ND | ND | 28.66 |
| 0.5 | ND | ND | 35.56 | 28.86 |
| 0.05 | ND | ND | 38.56 | 28.79 |
| 0.05 | ND | ND | 38.44 | 28.84 |
| 0.05 | ND | ND | 38.93 | 28.84 |
| 0.005 | ND | ND | 39.66 | 28.7 |
| 0.005 | ND | ND | ND | 28.78 |
| 0.005 | ND | ND | ND | 28.72 |
| NTC | ND | ND | ND | ND |
| EC | ND | ND | ND | 28.42 |
| SARS PTV | 30.50 | 35.07 | ND | 33.44 |
| RSV PTC | ND | ND | 17.07 | 25.20 |

Limit of Detection Confirmation Study. The limit of detection for the AvellinoCoV2 Respiratory Panel was confirmed in the most complex specimen matrix, which is the pooled iSWAB samples. The pooled iSWAB sample was spiked with BEI extracted genome viral RNA at the estimated limit of detection concentrations of 1 copy/uL. The spiked matrix was extracted 20 times and were run on the ABI 7500 Fast Real Time PCR System. The results are presented in the tables below:

TABLE 22
LoD 20 Replica Data Summary

| | Virus quantity: 1 copy/uL | | | |
|---|---|---|---|---|
| | Influenza | Influenza | Influenza | |
| 20 | A | B | C | RP |
| Replicates | Ct | Ct | Ct | Ct |
| 1 | 34.12 | 35.29 | 34.69 | 28.91 |
| 2 | 35.22 | 35.16 | 35.18 | 29.24 |
| 3 | 34.16 | 34.98 | 35.01 | 29.17 |
| 4 | 35.07 | 35.03 | 34.85 | 29.74 |
| 5 | 35.03 | 35.07 | 35.35 | 29.94 |
| 6 | 34.32 | 35.17 | 35.29 | 29.63 |
| 7 | 35.44 | 35.03 | 34.67 | 29.98 |
| 8 | 34.31 | 35.29 | 34.47 | 29.49 |
| 9 | 34.84 | 35.55 | 36.67 | 29.38 |
| 10 | 34.11 | 35.16 | 35.70 | 29.57 |
| 11 | 33.81 | 35.20 | 36.33 | 29.37 |
| 12 | 34.53 | 34.94 | 34.44 | 29.55 |
| 13 | 35.93 | 34.87 | 34.93 | 29.56 |
| 14 | 34.08 | 35.06 | 34.71 | 29.69 |
| 15 | 33.82 | 34.83 | 34.81 | 29.59 |
| 16 | 33.18 | 35.40 | 34.28 | 29.96 |
| 17 | 34.21 | 35.19 | 35.50 | 30.29 |
| 18 | 34.27 | 34.99 | 34.20 | 29.94 |
| 19 | 34.72 | 35.03 | 34.02 | 30.45 |
| 20 | 33.93 | 35.06 | 33.76 | 30.31 |
| Avg. | 34.46 | 35.11 | 34.94 | 29.69 |
| SD | 0.65 | 0.18 | 0.73 | 0.40 |
| Call rate | 20/20 | 20/20 | 20/20 | 20/20 |
| NTC | ND* | ND | ND | ND |
| EC | ND | ND | ND | 27.51 |
| PTC | 22.32 | 26.26 | 23.50 | 27.44 |

*ND: Not Detected

Analytical Sensitivity is determined to be 100% with the LoD of 5 copy/uL.

$$\\ \begin{matrix}Analytical Sensitivity = TP / ( TP + FN ) \times 100 \% \\ = 20 samples / ( 20 samples ) \times 100 \% = 100 \%\end{matrix}$$

The data demonstrated 100% analytical sensitivity. Target performance is ≥95% analytical sensitivity for all samples tested. Additionally, per FDA policy, 20 out of 20 replicates are positive at LoD of 1 copy/uL. Therefore, Limit of Detection target performance was achieved. It is concluded that the Limit of Detection and Analytical Sensitivity performance characteristic is validated and have met acceptance criteria for this testing system.

### Performance Characteristics Summary: In Silico Inclusivity

Utilizing the NCBI BLAST (Ye, 2012) and blastn suite, the Influenzas A, B, and C forward and reverse primers and probes were blast against the known influenza sequences published in NCBI. The primer and probe set targets are able to capture all influenza stains, in silico. Therefore, the in silico Inclusivity study confirms that there was 100% detection of all Influenza A, B and C strains. Please see the BLAST result reports attached: Influenza In silico Inclusivity Cross-Reactivity. Inclusivity target performance was achieved.

Performance Characteristics Summary: In Silico Cross-Reactivity Avellino first found the above organisms' Taxonomy ID by browsing NCBI's Taxonomy Browser. Then the specific forward and reverse primers were blast with NCBI's Primer-BLAST (Ye, 2012). The probes were blast with NCBI's Nucleotide BLAST. None of the primers or probes have any cross-reactivity with the organisms listed.

TABLE 23
NCBI BLAST Results
NCBI BLAST Results

| | Influenza | Influenza | |
|---|---|---|---|
| High priority | A, B, C | A, B, C | Influenza |
| organisms likely in | Forward | Reverse | A, B, C |
| circulating areas | Primers | Primers | Probes |
| Adenovirus | No target | No target | No target |
| (e.g. C1 Ad. 71) | templates | templates | templates |
| Human | No target | No target | No target |
| Metapneumovirus | templates | templates | templates |
| (hMPV) | | | |
| Parainfluenza virus 1-4 | No target | No target | No target |
| | templates | templates | templates |
| Enterovirus (e.g. EV68) | No target | No target | No target |
| | templates | templates | templates |
| Respiratory syncytial | No target | No target | No target |
| virus | templates | templates | templates |
| Rhinovirus | No target | No target | No target |
| | templates | templates | templates |
| Chlamydia pneumoniae | No target | No target | No target |
| | templates | templates | templates |
| Haemophilus influenzae | No target | No target | No target |
| | templates | templates | templates |
| Legionella pneumophila | No target | No target | No target |
| | templates | templates | templates |
| Mycobacterium | No target | No target | No target |
| tuberculosis | templates | templates | templates |
| Streptococcus | No target | No target | No target |
| pneumoniae | templates | templates | templates |
| Streptococcus pyogenes | No target | No target | No target |
| | templates | templates | templates |
| Bordetella pertussis | No target | No target | No target |
| | templates | templates | templates |
| Mycoplasma | No target | No target | No target |
| pneumoniae | templates | templates | templates |
| Pneumocystis jiroveci | No target | No target | No target |
| (PJP) | templates | templates | templates |
| Pooled human nasal | N/A | N/A | N/A |
| wash-to represent | | | |
| diverse microbial flora | | | |
| in the human respiratory | | | |
| tract | | | |
| Candida albicans | No target | No target | No target |
| | templates | templates | templates |
| Pseudomonas | No target | No target | No target |
| aeruginosa | templates | templates | templates |
| Staphylococcus | No target | No target | No target |
| epidermis | templates | templates | templates |
| Streptococcus salivarius | No target | No target | No target |
| | templates | templates | templates |
| | Influenzas | Influenzas | |
| Other high priority | A B, C | A, B, C | Influenzas |
| pathogens from the | Forward | Reverse | A, B, C |
| same genetic family | Primers | Primers | Probes |
| Other Influenza virus | No target | No target | No target |
| strains | templates | templates | templates |

Cross reactivity target performance was achieved.

### Performance Characteristics Summary: Quality Control

NTC, EC, and PTC controls were verified to amplify as expected. RP samples are positive at or before 38 cycles for all patient samples and EC. Ct is less than 40 cycles for samples.

TABLE 24
Validation Performance Characteristics Summary

| Performance Characteristic | Conclusion |
|---|---|
| Accuracy | This validation demonstrated 100% accuracy. Target |
| | performance goal is ≥95%; therefore, the AvellinoCoV2 |
| | Respiratory Panel has passed validation for the Accuracy |
| | performance characteristic. |
| Precision | This validation demonstrated 100% concordance. Target |
| | performance goal is ≥95%; therefore, the AvellinoCoV2 |
| | Respiratory Panel has passed validation for the Precision |
| | performance characteristic. |
| Analytical Sensitivity (LoD and | This validation demonstrated 100% analytical sensitivity. |
| in silico Inclusivity) | Target performance goal is ≥95%; therefore, |
| | AvellinoCoV2 Respiratory Panel has passed validation |
| | for the Analytical Sensitivity performance characteristic. |
| | In our study, there were no false negatives found. The |
| | LoD has been determined to be 1 copies/uL. It also met |
| | FDA policy of 19/20 positive samples passing, where |
| | Avellino has achieved 20/20 at LoD of 1 copy/uL level. |
| | In silico inclusivity of the Influenza A, B and C target |
| | performance was achieved. The in silico inclusivity of |
| | SARS-COV2 was achieved and included in EUA20006. |
| Analytical Specificity (in silico | in silico cross-reactivity of Influenza A, B and C target |
| Cross-reactivity) | performance was achieved. The in silico inclusivity of |
| | SARS-COV2 was achieved and included in EUA20006. |
| Quality Control | NTC, EC, and PTC controls were verified to amplify as |
| | expected. RP samples are positive at or before 38 cycles |
| | for all patient samples and EC. Ct is less than 40 cycles |
| | for samples. |

The following virus targets were validated:
SARS-CoV2
SARS-like Coronavirus
Influenza A
Influenza B
Influenza C
RSV

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A composition comprising at least one probe selected from the group consisting of probe 1, probe 2, and probe 3,
wherein probe 1 comprises a nucleic acid sequence having at least 85% sequence identity to SEQ ID NO: 7,
wherein probe 2 comprises a nucleic acid sequence having at least 85% sequence identity to SEQ ID NO: 9, and
wherein probe 3 comprises a nucleic acid sequence having at least 85% sequence identity to SEQ ID NO: 10.

2. The composition according to claim 1, wherein the composition comprises probe 1, probe 2, and probe 3.

3. The composition according to claim 1, wherein the composition further comprises probe 4, wherein probe 4 comprises a nucleic acid sequence having at least 85% sequence identity to SEQ ID NO: 11.

4. The composition according to claim 1, further comprising at least one forward primer selected from the group consisting of forward primers comprising a nucleic acid sequence having at least 85% sequence identity to SEQ ID NO: 18, 20, or 21.

5. The composition according to claim 4, wherein the composition comprises a first forward primer comprising a nucleic acid sequence having at least 85% sequence identity to SEQ ID NO: 18, a second forward primer comprising a nucleic acid sequence having at least 85% sequence identity to SEQ ID NO: 20, and a third forward primer comprising a nucleic acid sequence having at least 85% sequence identity to SEQ ID NO: 21.

6. The composition according to claim 5, wherein the composition further comprises a fourth forward primer comprising a nucleic acid sequence having at least 85% sequence identity to SEQ ID NO: 22.

7. The composition according to claim 1, further comprising at least one reverse primer selected from the group consisting of reverse primers comprising a nucleic acid sequence having at least 85% sequence identity to SEQ ID NO: 29, 31, or 32.

8. The composition according to claim 7, wherein the composition comprises a first reverse primer comprising a nucleic acid sequence having at least 85% sequence identity to SEQ ID NO: 29, a second reverse primer comprising a nucleic acid sequence having at least 85% sequence identity to SEQ ID NO: 31, and a third reverse primer comprising a nucleic acid sequence having at least 85% sequence identity to SEQ ID NO: 32.

9. The composition according to claim 8, wherein the composition further comprises a fourth reverse primer comprising a nucleic acid sequence having at least 85% sequence identity to SEQ ID NO: 32.

10. The composition according to claim 1, further comprising at least one dye selected from the group consisting of JUN-QSY, ABY-QSY, FAM, VIC, and Cy5.

11. The composition according to claim 1,
wherein probe 1 comprises a nucleic acid sequence having 100% sequence identity to SEQ ID NO: 7,
wherein probe 2 comprises a nucleic acid sequence having 100% sequence identity to SEQ ID NO: 9, and
wherein probe 3 comprises a nucleic acid sequence having 100% sequence identity to SEQ ID NO: 10.

12. A method for detecting a respiratory illness, comprising: hybridizing a target nucleotide molecule in a sample with the composition of claim 1.

13. The method according to claim 12, wherein the respiratory illness is selected from the group consisting of MERS and COVID-19.

14. The method according to claim 12, wherein the sample is obtained from a bodily fluid of a subject.

15. The method according to claim 14, wherein the bodily fluid is saliva, a nasopharyngeal fluid, an oropharyngeal fluid, a mid-turbinate fluid, or blood.

16. The method according to claim 14, wherein the subject is human.

17. A method for treating a respiratory illness, comprising detecting the respiratory illness according to claim 12.
